(19) United States
(12) Patent Application Publication
(10) Pub. No.: US 2025/0352323 A1
(43) Pub. Date: Nov. 20, 2025

(54) METHODS AND SYSTEMS FOR TREATING PULMONARY DISEASE

(71) Applicant: Apreo Health, Inc., Menlo Park, CA (US)

(72) Inventors: Patrick P. Wu, San Carlos, CA (US); Claudia Lynn Pham, San Francisco, CA (US); Brandon Wai-lon Chu, San Francisco, CA (US); Tony Le, Rohnert Park, CA (US); Jagannath Padmanabhan, San Carlos, CA (US); Steven W. Kim, Los Altos, CA (US); Nifer Beth Goldman, Redwood City, CA (US); Martin L. Mayse, Wayzata, MN (US); Karun D. Naga, Los Altos, CA (US); Huy Hoang Tran, San Jose, CA (US)

(21) Appl. No.: 19/279,977

(22) Filed: Jul. 24, 2025

Related U.S. Application Data
(63) Continuation of application No. PCT/US2024/013012, filed on Jan. 25, 2024.
(60) Provisional application No. 63/441,167, filed on Jan. 25, 2023.

(51) Int. Cl. A61F 2/04; A61B 1/267; A61F 2/86

(52) U.S. Cl. CPC A61F 2/04; A61B 1/2676; A61F 2/86; A61F 2002/043; A61F 2250/0093; A61F 2250/0096; A61F 2250/0098

(57) ABSTRACT

Endobrochial implants and delivery systems therefor are disclosed herein. In some embodiments, a delivery system can be used for deploying an implant at a treatment location, where the implant includes a tubular region with one or more interstitial regions. A delivery system can, for example include a flexible elongate member having an implant mounting surface, wherein the implant mounting surface comprises a conformable material configured to adapt to the one or more interstitial regions of the implant when the implant is radially collapsed on the implant mounting surface, thereby engaging the implant. The delivery system can also include a sheath at least partially covering the elongate member, wherein the sheath is movable relative to the elongate member to at least partially expose the implant mounting surface.

### CROSS-REFERENCE TO RELATED APPLICATION

This application is a continuation of International Application No. PCT/US2024/013012, filed Jan. 25, 2024, which claims the benefit of priority to U.S. Patent Application No. 63/441,167, filed Jan. 25, 2023, each of which is incorporated herein by reference in its entirety.

### TECHNICAL FIELD

The present technology relates to implants, such as endobronchial implants for treating chronic obstructive pulmonary disorder.

### BACKGROUND

Chronic obstructive pulmonary disorder (COPD) is a disease of impaired lung function. Symptoms of COPD include coughing, wheezing, shortness of breath, and chest tightness. Cigarette smoking is the leading cause of COPD, but long-term exposure to other lung irritants (e.g., air pollution, chemical fumes, dust, etc.) may also cause or contribute to COPD. In most cases, COPD is a progressive disease that worsens over the course of many years. Accordingly, many people have COPD, but are unaware of its progression. COPD is currently a major cause of death and disability in the United States. Severe COPD may prevent a patient from performing even basic activities such as walking, climbing stairs, or bathing. Unfortunately, there is no known cure for COPD. Nor are there known medical techniques capable of reversing the pulmonary damage associated with COPD.

In normal respiration, the act of inhaling draws air into the lungs via the nose or mouth and the trachea. Within each lung, inhaled air moves into a branching network of progressively narrower airways called bronchi, and then into the narrowest airways called bronchioles. The bronchioles end in bunches of tiny round structures called alveoli. Small blood vessels called capillaries run through the walls of the alveoli. When inhaled air reaches the alveoli, oxygen moves from the alveoli into blood in the capillaries. At the same time, carbon dioxide moves in the opposite direction, i.e., from blood in the capillaries into the alveoli. This process is called gas exchange. In a healthy lung, the airways and alveoli are elastic and stretch to accommodate air intake. When a breath is drawn in, the alveoli fill up with air like small balloons. When a breath is expelled, the alveoli deflate. This expansion of the alveoli is an important part of effective gas exchange. Alveoli that are free to expand exchange more gas than alveoli that are inhibited from expanding.

In COPD-affected lung tissue, less air flows through the airways for a variety of reasons. The airways and/or alveoli may be relatively inelastic, the walls between the alveoli may be damaged or destroyed, the walls of the airways may be thick or inflamed, and/or the airways may generate excessive mucus resulting in mucus buildup and airway blockage. In a typical case of COPD, the disease does not equally affect all airways and alveoli in a lung. A lung may have some regions that are significantly more affected than other regions. In severe cases, the airways and alveoli that are unsuitable for effective gas exchange may make up 20 to 30 percent or more of total lung volume.

The effects of COPD are often most pronounced when a patient exercises or engages in other physical exertion that would cause a healthy person to breath heavily. A patient with COPD may not be able to breathe heavily because diseased portions of the patient's lungs trap air, resulting in an inability to exhale completely. This, in turn, inhibits subsequent expansion of healthy lung tissue. Thus, during exercise or other physical exertion, the lungs of a COPD patient may operate in a state of dynamic hyperinflation that impairs respiratory mechanics and increases the work of breathing. Hyperinflation of the lungs may also hinder cardiac filling, lead to dyspnea, and/or reduce a patent's exercise performance. These and/or other detrimental effects of COPD can lead to a cascade of symptoms that eventually impairs a patient's quality of life and increases the risk of severe disability and death.

The term COPD includes both chronic bronchitis and emphysema. About 25% of COPD patients have emphysema. About 40% of these emphysema patients have severe emphysema. Furthermore, it is common for COPD patients to have symptoms of both chronic bronchitis and emphysema. In chronic bronchitis, the lining of the airways is inflamed, generally as a result of ongoing irritation. This inflammation results in thickening of the airway lining and in production of a thick mucus that may coat and eventually congest the airways. Emphysema, in contrast, is primarily a pathological diagnosis concerning abnormal permanent enlargement of air spaces distal to the terminal bronchioles. In emphysematous lung tissue, the small airways and/or alveoli typically have lost their structural integrity and/or their ability to maintain an optimal shape. For example, damage to or destruction of alveolar walls may have resulted in fewer, but larger alveoli. This may significantly impair normal gas exchange. Within the lung, focal or “diseased” regions of emphysematous lung tissue characterized by a lack of discernible alveolar walls may be referred to as pulmonary bullae. These relatively inelastic pockets of dead space are often greater than 1 cm in diameter and do not contribute significantly to gas exchange. Pulmonary bullae tend to retain air and thereby create hyperinflated lung sections that restrict the ability of healthy lung tissue to fully expand upon inhalation. Accordingly, in patients with emphysema, not only does the diseased lung tissue no longer contribute significantly to respiratory function, it impairs the functioning of healthy lung tissue.

Pharmacological treatment is often prescribed for COPD. A treatment algorithm of bronchodilators, B2-agonists, muscarinic agonists, corticosteroids, or combinations thereof may provide short term alleviation of the symptoms of COPD. These treatments, however, do not cure COPD or meaningfully slow the disease progression. Non-pharmaceutical management solutions, such as home oxygen, non-invasive positive pressure ventilation, and pulmonary rehabilitation, are also common but have only modest therapeutic effect. Another treatment option for patients with severe emphysema is lung volume reduction surgery (LVRS). This surgery involves removing poorly functioning portions a lung (typically up to 20 to 25 percent of lung volume) thereby reducing the overall size of the lung and making more volume within the chest cavity available for expansion of relatively healthy lung tissue. With greater available volume for expansion, the lung tissue remaining after LVRS has an enhanced capacity for effective gas exchange. The obvious drawback of LVRS is its highly invasive nature. Accordingly, LVRS is usually considered to be a last-resort option suitable for only a small percentage of emphysema patients.

Procedures for lung volume reduction without surgical removal of diseased lung tissue also exist. Examples include use coils or clips to seize and physically compact diseased lung tissue. These procedures can reduce the overall volume of a lung for an effect similar to that of LVRS. The potential of these procedures is limited, however, because the proximal positioning of the coils or clips tends to isolate not just diseased portions of the lung, but also healthy portions. Furthermore, these procedures are often associated with serious complications such as pneumothorax and chronic increased risk of respiratory infections.

Another device-based treatment for COPD involves placement of one-directional stent valves in airways proximal to emphysematous tissue. These valves allow air to flow out of but not into overinflated portions of the lung. This approach is only recommended for patients with little to no collateral ventilation (i.e., ventilation of alveoli via pathways that bypass normal airways). Unfortunately, fewer than 20% of patients with emphysema lack collateral ventilation. Accordingly, one-directional stent valves are not suitable for most emphysema patients. Moreover, as with endobronchial coils and clips, the proximal positioning of one-directional stent valves can isolate not just diseased portions of the lung, but also healthy portions.

Bronchoscopic thermal vapor ablation (BTVA) is yet another suboptimal COPD treatment option. BTVA involves introducing heated water vapor into diseased lung tissue. This produces a thermal reaction leading to an initial localized inflammatory response followed by permanent fibrosis and atelectasis. Similar to thermal treatments like BTVA, there are also biochemical treatments that involve injecting glues or sealants into diseased lung tissue. Both thermal and biochemical procedures may precipitate remodeling that results in reduction of tissue and air volume at targeted regions of hyperinflated lung. These procedures, however, are known to cause local toxicity and associated complications that undermine their potential therapeutic benefit.

Although not conventionally used to treat COPD, stents are sometimes used in the lumen of the central airways (i.e., the trachea, main bronchi, lobar bronchi, and/or segmental bronchi) to temporarily improve patency of these airways. For example, stents may be used to temporarily improve patency in a central airway affected by a benign or malignant obstruction. Central airway stenting in not an effective treatment for emphysema because central airways have little or no impact on the overall airway obstruction and/or airway narrowing associated with emphysema. Furthermore, conventional stents, when placed in airways, are plagued by issues of occlusion, including the formation of granulation tissue and mucous impaction.

Some other known COPD treatments involve bypassing an obstructed airway. For example, a perforation through the chest wall into the outer portions of the lung can be used to create a direct communication (i.e., a bypass tract) between diseased alveoli and the outside of the body. If no other steps are taken, these bypass tracts will close by normal healing or by the formation of granulation tissue, thereby eliminating the therapeutic benefit. Placing a tubular prosthetic in the bypass tract can temporarily extend the therapeutic benefit. Such prosthetics, however, eventually induce a foreign body reaction and accelerate the formation of granulation tissue. Moreover, forming bypass tracts tends to be difficult and time intensive. Once formed, bypass tracts can also be uncomfortable, inconvenient, and/or debilitating for the patient.

COPD is a major public health issue. There are over one million patients in the United States alone with severe emphysema and severe hyperinflation. An overwhelming majority of these patients are underserved by currently available treatments. The global unmet clinical need, including in countries with high incidence of respiratory disease due to smoking, is many times greater than in the United States. As discussed above, conventional approaches to treating COPD are associated with serious complications, have limited effectiveness, are only suitable for a small percentage of COPD patients, and/or have other significant disadvantages. Given the prevalence of the disease and the inadequacy of conventional treatments, there is a great need for innovation in this field.

### SUMMARY

The present technology is illustrated, for example, according to various aspects described below, including with reference to the figures. Various examples of aspects of the present technology are described in this summary section as Examples numbered (1, 2, 3, etc.) for convenience. These are provided as examples and are not intended to limit the present technology.

Example 1. A delivery system for deploying an implant at a treatment location, wherein the implant comprises a tubular region with one or more interstitial regions, the delivery system comprising:
a flexible elongate member having an implant mounting surface, wherein the implant mounting surface comprises a conformable material configured to adapt to the one or more interstitial regions of the implant when the implant is radially collapsed on the implant mounting surface, thereby engaging the implant; and
a sheath at least partially covering the elongate member, wherein the sheath is movable relative to the elongate member to at least partially expose the implant mounting surface.

Example 2. The delivery system of example 1, wherein the conformable material extends fully circumferentially around a longitudinal axis of the elongate member.

Example 3. The delivery system of example 1 or 2, wherein the implant mounting surface is substantially smooth.

Example 4. The delivery system of any one of examples 1-3, wherein the conformable material comprises a thermoplastic.

Example 5. The delivery system of any one of examples 1-4, wherein the conformable material has a durometer between about 5A and about 75A.

Example 6. The delivery system of any one of examples 1-5, wherein the implant mounting surface comprises a first segment comprising the conformable material and a second segment comprising the conformable material.

Example 7. The delivery system of example 6, wherein the first and second segments are axially spaced apart along a longitudinal axis of the elongate member.

Example 8. The delivery system of example 6 or 7, wherein the first and second segments are circumferentially spaced apart around a longitudinal axis of the elongate member. Example 9. The delivery system of any one of examples 1-8, further comprising a handle operably coupled to the sheath.

Example 10. The delivery system of example 9, wherein the handle comprises an actuator configured to move the sheath relative to the elongate member.

Example 11. The delivery system of example 9 or 10, wherein the handle comprises a lock configured to selectively fix an axial position of the sheath relative to the elongate member.

Example 12. The delivery system of any one of examples 1-11, wherein the elongate member comprises an atraumatic tip.

Example 13. The delivery system of any one of examples 1-12, wherein the sheath is a first sheath and the delivery system further comprises a second sheath at least partially covering the first sheath, wherein the second sheath is movable relative to the first sheath

Example 14. The delivery system of any one of examples 1-13, further comprising a second lock configured to couple the delivery system to a bronchoscope

Example 15. The delivery system of example 14, wherein the second lock is configured to selectively limit an axial position of the elongate member relative to the bronchoscope.

Example 16. A system comprising:
an implant comprising a tubular region with one or more interstitial regions; and
a delivery system comprising:
a flexible elongate member having an implant mounting surface, wherein the mounting surface comprises a conformable material; and
a sheath at least partially covering the elongate member, wherein the sheath is movable relative to the elongate member to at least partially expose the implant mounting surface,
wherein the implant is arranged on the mounting surface such that the conformable material adapts to the one or more interstitial regions, thereby engaging the implant.

Example 17. The system of example 16, wherein the implant is configured to transition from a radially compressed configuration to a radially expanded configuration.

Example 18. The system of example 17, wherein the implant is configured to transition from the radially compressed configuration to a radially expanded configuration without experiencing a change in implant length.

Example 19. The system of example 17 or 18, wherein the implant is configured to self-expand from the radially compressed configuration to the radially expanded configuration.

Example 20. The system of any one of examples 16-19, wherein the tubular region comprises a wire extending along a continuous wire path turning about a longitudinal axis of the implant.

Example 21. The system of any one of examples 16-20, wherein the one or more interstitial regions comprises an open helical region.

Example 22. The system of any one of examples 16-21, wherein the conformable material extends fully circumferentially around a longitudinal axis of the elongate member.

Example 23. The system of any one of examples 16-22, wherein the implant mounting surface is substantially smooth.

Example 24. The system of any one of examples 16-23, wherein the conformable material comprises a thermoplastic.

Example 25. The system of any one of examples 16-24, wherein the conformable material has a durometer between about 5A and about 75A.

Example 26. The system of any one of examples 16-25, wherein the conformable material is configured to engage with at least one of a proximal portion and a distal portion of the implant.

Example 27. The system of any one of examples 16-26, wherein the conformable material is configured to engage with an entire length of the implant.

Example 28. The system of any one of examples 16-27, wherein the implant mounting surface is a first implant mounting surface, and the elongate member comprises a second implant mounting surface comprising a second conformable material.

Example 29. The system of example 28, wherein the first and second implant mounting surfaces are axially spaced apart along a longitudinal axis of the elongate member.

Example 30. The system of any one of examples 16-29, wherein the delivery system further comprises a handle operably coupled to the sheath.

Example 31. The system of example 30, wherein the handle comprises an actuator configured to move the sheath relative to the elongate member.

Example 32. The system of example 30 or 31, wherein the handle comprises a lock configured to selectively fix an axial position of the sheath relative to the elongate member.

Example 33. The system of any one of examples 16-32, wherein the elongate member comprises an atraumatic tip.

Example 34. The system of any one of examples 16-33, wherein the sheath is a first sheath and the delivery system further comprises a second sheath at least partially covering the first sheath, wherein the first sheath is movable relative to the second sheath.

Example 35. The system of any one of examples 16-34, wherein the delivery system comprises a second lock configured to couple the delivery system to a bronchoscope.

Example 36. The system of example 35, wherein the second lock is configured to selectively limit movement of the elongate member relative to the bronchoscope.

Example 37. The system of any one of examples 16-36, further comprising a single-use bronchoscope.

Example 38. A method for deploying an implant in a patient, wherein the implant comprises a tubular region with one or more interstitial regions and is configured to transition from a radially compressed configuration to a radially expanded configuration, the method comprising:
advancing, in the patient, a flexible elongate member having an implant mounting surface comprising a conformable material, wherein the implant is arranged on the mounting surface in the radially compressed configuration such that the conformable material adapts to the one or more interstitial regions, wherein the elongate member is at least partially covered by a sheath that is movable relative to the elongate member;
positioning a first end of the compressed implant at a first target location;
moving the sheath relative to the elongate member, thereby exposing the implant; and
allowing the implant to transition to the radially expanded configuration.

Example 39. The method of example 38, wherein advancing the elongate member in the patient comprises advancing the elongate member through a working channel of a bronchoscope.

Example 40. The method of example 39, further comprising selectively limiting movement of the elongate member relative to the bronchoscope while moving the sheath relative to the elongate member in the patient.

Example 41. The method of example 40, wherein selectively limiting movement of the sheath comprises limiting an axial position of the elongate member relative to the bronchoscope.

Example 42. The method of any one of examples 39-41, further comprising allowing rotational movement of the sheath relative to the bronchoscope.

Example 43. A delivery system for deploying an implant at a treatment location, the delivery system comprising:
a flexible elongate member having an implant mounting surface, wherein the implant mounting surface comprises an engagement means for securing the implant when the implant is radially collapsed on the implant mounting surface; and
a sheath at least partially covering the elongate member, wherein the sheath is movable relative to the elongate member to at least partially expose the implant mounting surface.

Example 44. The delivery system of example 43, wherein the engagement means comprises a conformable material configured to adapt to one or more interstitial regions of the implant when the implant is radially collapsed on the implant mounting surface, thereby engaging the implant.

Example 45. The delivery system of example 44, wherein the conformable material comprises a thermoplastic.

Example 46. The delivery system of any one of examples 43-45, wherein the engagement means comprises a bioadhesive.

Example 47. The delivery system of example 46, wherein the bioadhesive comprises at least one of the group consisting of a synthetic polymer, a polysaccharide, cellulose, chitosan, and fibrin.

Example 48. The delivery system of any one of examples 43-47, wherein the engagement means comprises a textured surface.

Example 49. The delivery system of any one of examples 43-48, wherein the engagement means extends fully circumferentially around a longitudinal axis of the elongate member.

Example 50. An implant configured to be deployed at a treatment location within a bronchial tree of a patient, the implant comprising:
a tubular structure comprising a wire extending along a continuous wire path, the tubular structure comprising:
a proximal end portion configured to be deployed at a first airway of the bronchial tree and exert a first chronic outward force; and
a distal end portion configured to be deployed at a second airway of the bronchial tree and exert a second chronic outward force, wherein a generation of the second airway is greater than a generation of the first airway,
wherein the second chronic outward force is greater than the first chronic outward force.

Example 51. The implant of example 50, wherein the tubular structure comprises an intermediate portion between the proximal end portion and the distal end portion, wherein the tubular structure is configured to exert a variable chronic outward force along its length.

Example 52. The implant of example 50 or 51, wherein the variable chronic outward force ranges from the first chronic outward force to the second chronic outward force.

Example 53. The implant of any one of examples 50-52, wherein the second chronic outward force is between about two and about four times greater than the first chronic outward force.

Example 54. The implant of any one of examples 50-53, wherein the second chronic outward force is about three times greater than the first chronic outward force.

Example 55. The implant of any one of examples 50-54, wherein the second chronic outward force is about 15N, and the first chronic outward force is about 5N.

Example 56. The implant of any one of examples 50-55, wherein the tubular structure is configured to transition from a radially compressed configuration to a radially expanded configuration.

Example 57. The implant of any one of examples 50-56, wherein the second chronic outward force is sufficient to cause dilation of the second airway to at least 2 times the native diameter of the second airway.

Example 58. A system for selecting a length of an endobronchial implant to be placed in a patient, the system comprising:
a flexible elongate member configured to be inserted through a lumen of a device,
wherein the elongate member comprises a plurality of markers arranged along a longitudinal axis of the elongate member, wherein at least a portion of the plurality of markers are evenly distributed along the longitudinal axis, or at least a portion of the plurality of markers correspond to predetermined available lengths of the endobronchial implant, or both.

Example 59. The system of example 58, wherein the plurality of markers are arranged on a distal portion of the elongate member.

Example 60. The system of example 58, wherein the plurality of markers are arranged on a proximal portion of the elongate member.

Example 61. The system of any one of examples 58-60, wherein at least a portion of the plurality of markers comprise a radiopaque material.

Example 62. The system of any one of examples 58-61, wherein the elongate member is a guidewire.

Example 63. The system of any one of examples 58-61, wherein the elongate member is a probe.

Example 64. The system of any one of examples 58-61, wherein the elongate member is a sheath defining a lumen.

Example 65. The system of example 64, wherein the elongate member is a first elongate member and the sheath is a first sheath, wherein the first sheath is coupleable to an implant delivery system comprising:
a second flexible elongate member having an implant mounting surface; and
a second sheath at least partially covering the second elongate member, wherein the second sheath is movable relative to the second elongate member to at least partially expose the implant mounting surface.

Example 66. The system of example 65, wherein the first sheath is removably coupleable to a handle of the implant delivery system.

Example 67. The system of any one of examples 58-F9, wherein the elongate member comprises a proximity sensor.

Example 68. A method for selecting a length of an endobronchial implant to be placed in a patient, the method comprising:
navigating a device toward a target airway in the patient;
advancing an elongate member through a lumen of the device such that a distal end of the elongate member is distally beyond a distal end of the device;
measuring a length of the target airway using a plurality of markers on the elongate member; and
selecting a length of an endobronchial implant based on the measured length of the target airway.

Example 69. The method of example 68, wherein the device is a bronchoscope or robotic catheter.

Example 70. The method of example 69, wherein the device is a single-use bronchoscope.

Example 71. The method of any one of examples 68-70, wherein advancing the elongate member comprises advancing the elongate member until the distal end of the elongate member is located at a desired location of a distal end of the endobronchial implant.

Example 72. The method of example 71, wherein the elongate member is a guidewire.

Example 73. The method of any one of examples 68-70, wherein advancing the elongate member comprises advancing the elongate member until the distal end of the elongate member is adjacent to pleura.

Example 74. The method of example 73, further comprising proximally retracting the elongate member until the distal end of the elongate member is located at a desired location of a distal end of the endobronchial implant.

Example 75. The method of any one of examples 68-74, wherein the plurality of markers are arranged on a distal portion of the elongate member.

Example 76. The method of any one of examples 68-75, wherein the plurality of markers are arranged on a proximal portion of the elongate member.

Example 77. The method of any one of examples 68-76, wherein at least a portion of the plurality of markers are evenly distributed along a longitudinal axis of the elongate member.

Example 78. The method of any one of examples 68-77, wherein at least a portion of the plurality of markers correspond to predetermined available lengths of the endobronchial implant.

Example 79. The method of any one of examples 68-78, wherein at least a portion of the plurality of markers comprise a radiopaque material.

Example 80. The method of any one of examples 68-79, further comprising measuring a distance between the distal end of the elongate member and pleura.

Example 81. The method of example 80, wherein the distance between the distal end of the elongate member and pleura is measured with a sensor.

Example 82. A delivery system for deploying an implant at a treatment location, comprising:
a flexible elongate member having an implant mounting surface configured to receive the implant;
a sheath at least partially covering the elongate member, wherein the sheath is movable relative to the elongate member to at least partially expose the implant mounting surface; and
a sensor configured to detect proximity of a distal end of the delivery system to a tissue wall.

Example 83. The delivery system of example 82, wherein the sensor is arranged on a distal end of the elongate member.

Example 84. The delivery system of example 82, wherein the sensor is arranged on a distal end of the sheath.

Example 85. The delivery system of any one of examples 82-84, wherein the sensor comprises an ultrasonic sensor.

Example 86. The delivery system of any one of examples 82-85, wherein the sensor comprises an infrared sensor.

Example 87. A method for deploying an implant at a treatment location, the method comprising:
advancing, in an airway of the patient, a delivery system comprising a flexible elongate member having an implant mounted thereon, a sheath that is movable relative to the elongate member, and a proximity sensor arranged on a distal end of the delivery system;
detecting a position of the distal end of the delivery system relative to a pleura of the patient using the proximity sensor;
positioning the implant at a desired target location without contacting the pleura with the delivery system; and
moving the sheath relative to the elongate member to expose the implant at the desired target location.

Example 88. The method of example 87, wherein advancing the delivery system comprises advancing the delivery system through a bronchoscope or robotic catheter.

Example 89. The method of example 88, wherein the bronchoscope is a single-use bronchoscope.

Example 90. The method of any one of examples 87-89, wherein moving the sheath to expose the implant allows the implant to transition from a radially compressed configuration to a radially expanded configuration.

Example 91. The method of any one of examples 87-90, wherein moving the sheath comprises proximally retracting the sheath.

Example 92. A method for deploying an implant at a treatment location, the method comprising:
advancing, in an airway of the patient, a delivery system comprising a flexible elongate member having an implant mounted thereon and a sheath that is movable relative to the elongate member;
advancing the delivery system until a distal end of the delivery system touches a pleura of the patient;
retracting the delivery system until the implant is at a desired target location; and
moving the she relative to the elongate member to expose the implant at the desired target location.

Example 93. The method of example 92, wherein advancing the delivery system comprises advancing the delivery system through a bronchoscope or robotic catheter.

Example 94. The method of example 93, wherein the bronchoscope is a single-use bronchoscope.

Example 95. The method of any one of examples 92-94, wherein moving the sheath to expose the implant allows the implant to transition from a radially compressed configuration to a radially expanded configuration.

Example 96. The method of any one of examples 92-95, wherein moving the sheath comprises proximally retracting the sheath.

### BRIEF DESCRIPTION OF THE DRAWINGS

Many aspects of the present technology can be better understood with reference to the following drawings. The relative dimensions in the drawings may be to scale with respect to some embodiments of the present technology. With respect to other embodiments, the drawings may not be to scale. The drawings may also be enlarged arbitrarily. For clarity, reference-number labels for analogous components or features may be omitted when the appropriate reference-number labels for such analogous components or features are clear in the context of the specification and all of the drawings considered together. Furthermore, the same reference numbers may be used to identify analogous components or features in multiple described embodiments.

FIG. 1 is a schematic illustration of a bronchial tree of a human subject within a chest cavity of the subject.

FIG. 2 is a schematic illustration of a bronchial tree of a human subject in isolation.

FIG. 3 is an enlarged view of a terminal portion of the bronchial tree shown in FIG. 2.

FIG. 4 is a table showing examples of dimensions and generation numbers of different portions of a bronchial tree of a human subject.

FIG. 5 is a diagram showing lung volumes during normal lung function.

FIG. 6 is a table showing airway wall composition at different portions of a bronchial tree of a human subject.

FIG. 7 is an anatomical illustration of airway wall composition at different portions of a bronchial tree of a human subject.

FIG. 8 is an anatomical illustration showing small airway narrowing in emphysematous lung tissue.

FIG. 9 is an anatomical illustration showing alveolar wall damage in emphysematous lung tissue.

FIG. 10 is an anatomical illustration showing normal airway patency during exhalation in healthy lung tissue.

FIG. 11 is an anatomical illustration showing airway collapse during exhalation in emphysematous lung tissue.

FIG. 12 is an anatomical illustration showing normal acinar.

FIG. 13 is an anatomical illustration showing centriacinar emphysema.

FIG. 14 is an anatomical illustration showing panacinar emphysema.

FIG. 15 is an anatomical illustration showing paraseptal emphysema.

FIG. 16 is a side view of an example implant.

FIG. 17 is a schematic end view of the implant shown in FIG. 16.

FIG. 18 is a side view of a portion of an implant within an airway.

FIG. 19 is a perspective view of an example implant.

FIG. 20 is a side view of a bend of the implant shown in FIG. 19.

FIG. 21 is a side view of a mandrel configured for use in manufacturing an implant.

FIG. 22 is a perspective view of the implant shown in FIG. 19 in a radially compressed state around a delivery member.

FIG. 23 is a perspective view of the implant shown in FIG. 19 in the radially compressed state shown in FIG. 22 with portions of the implant highlighted for finite element analysis.

FIG. 24A is a perspective view of an example delivery system. FIG. 24B is a detailed view of the delivery system shown in FIG. 24A.

FIGS. 25A and 25B are a top view and a side cross-sectional view, respectively, of an example handle of a delivery system.

FIGS. 25C-25F are illustrative schematics of example embodiments of user interface elements on an example handle of a delivery system.

FIGS. 26A and 26B are illustrative schematics of an example sheath actuator in an implant delivery system.

FIG. 27 is an illustrative schematic of an example rack and gear sheath actuator in an implant delivery system.

FIGS. 28A and 28B are top view and side view, respectively, of an illustrative schematic of an example pulley-based sheath actuator in an implant delivery system.

FIG. 29 is an illustrative schematic of an example telescoping sheath actuator in an implant delivery system.

FIG. 30 is an illustrative schematic of an example haptic feedback mechanism in an implant delivery system.

FIG. 31A is an illustrative schematic of an example elongate member in an implant delivery system. FIG. 31B is a detailed illustrative schematic of the elongate member shown in FIG. 31A.

FIG. 32 is an illustrative schematic of an example elongate member in an implant delivery system.

FIGS. 33A and 33B are illustrative schematics of an example elongate member and an example inner sheath in an implant delivery system.

FIG. 34A is a cross-sectional illustrative schematic view of an example implant delivery system. FIG. 34B is a detailed illustrative schematic of the delivery system shown in FIG. 34A.

FIG. 34C is an illustrative schematic of an implant delivery system with multiple segments of a conformable material for engaging an implant.

FIGS. 34D-34F are cross-sectional illustrative schematic views of different example embodiments of an elongate member in an implant delivery system.

FIG. 35 is a cross-sectional illustrative schematic view of an example implant delivery system.

FIG. 36 is a cross-sectional illustrative schematic view of an example elongate member and an example inner sheath in an implant delivery system.

FIG. 37 is an illustrative schematic of an example elongate member in an implant delivery system.

FIG. 38A is an illustrative schematic of an example inner sheath in an implant delivery system.

FIG. 38B is a cross-sectional illustrative schematic view of an example inner sheath in an implant delivery system.

FIGS. 39A and 39B are cross-sectional illustrative schematic views of various example inner sheath in an implant delivery system.

FIG. 40 depicts example braid and coil configurations for a structural reinforcement layer of an inner sheath in an implant delivery system.

FIG. 41 is an illustrative schematic of an example outer sheath in an implant delivery system.

FIG. 42 is an illustrative schematic of visualization markers on an example implant delivery device relative to a bronchoscope.

FIG. 43 is an illustrative schematic of a bronchoscope for use with implants and implant delivery systems.

FIGS. 44 through 50 are illustrative schematics of example locking arrangements for limiting movement of an implant delivery system relative to a bronchoscope.

FIG. 51 is an illustrative schematic of an example locking arrangement that permits limited axial movement of an implant delivery system relative to a bronchoscope.

FIGS. 52A and 52B are illustrative schematics of an example locking arrangement that permits limited axial movement of an implant delivery system relative to a bronchoscope.

FIGS. 53 and 54 are illustrations showing different respective times during deployment of an implant.

FIG. 55 is an anatomical illustration of an airway region at which an implant be deployed.

FIGS. 56-61 are partial schematic illustrations of different respective times during deployment of an implant at the airway region shown in FIG. 55.

FIG. 62 is an anatomical illustration of the airway region shown in FIG. 55 with certain native and expanded dimensions indicated.

FIG. 63 is a block diagram showing a method for improving pulmonary function in a human subject.

FIG. 64 is a diagram illustrating factors for consideration for an implant with a variable chronic outward force.

FIG. 65A is a perspective view of an implant in accordance with at least some embodiments of the present technology in an unconstrained state.

FIGS. 65B-65F are callouts corresponding to FIG. 65A.

FIG. 66A is an end view of the implant shown in FIG. 65A in the unconstrained state.

FIG. 66B is a callout corresponding to FIG. 66A.

FIG. 67 is a profile view of the implant shown in FIG. 65A in the unconstrained state.

FIG. 68 is a cross-sectional view of the implant shown in FIG. 65A in the unconstrained state taken along the line A-A in FIG. 67.

FIG. 69 is a cross-sectional view of the implant shown in FIG. 65A in the unconstrained state taken along the line B-B in FIG. 67.

FIG. 70 is a cross-sectional view of the implant shown in FIG. 65A in the unconstrained state taken along the line C-C in FIG. 67.

FIG. 71 is a cross-sectional view of the implant shown in FIG. 65A in the unconstrained state taken along the line D-D in FIG. 67.

FIG. 72 is a profile view of an implant in accordance with at least some embodiments of the present technology in an unconstrained state juxtaposed with a schematic diagram of portions of a wire path at an intermediate portion of the implant.

FIGS. 73A-74B are diagrams showing different respective subtended angles relevant to the implant shown in FIG. 72.

FIG. 75 is a profile view of the implant shown in FIG. 72 in a deployed state within an airway region.

FIG. 76 is a schematic diagram illustrating certain forces and dimensions relevant to implants in accordance with at least some embodiments of the present technology.

FIG. 77 is a schematic diagram illustrating a maximum distance between a point on an airway wall and a wire path of a simple coil.

FIG. 78 is a schematic diagram illustrating a maximum distance between a point on an airway wall and a wire path of an implant in accordance with at least some embodiments of the present technology.

FIGS. 79A-79D are illustrative schematics of an example sheath actuator in an implant delivery system.

FIGS. 80A and 80B are illustrative schematics of an example elongate member in first and second configurations, respectively.

FIG. 81A is an illustrative schematic of an example guide sheath in an implant delivery system. FIG. 81B is an illustrative schematic of an example inner sheath and handle in an implant delivery system.

FIG. 82 is an illustrative schematic of an example implant delivery system including a guide sheath, inner sheath, and handle in operation with a bronchoscope.

FIGS. 83A-83C are illustrative schematics of operation of an example guide sheath in an implant delivery system.

FIGS. 84A and 84B are illustrative schematics of operation of an example implant delivery system.

FIGS. 85A and 85B are illustrative schematics of operation of an example airway and implant sizing device.

FIGS. 86A and 86B are illustrative schematics of operation of an example airway and implant sizing device.

FIGS. 87A-87D are illustrative schematics of markers on an example airway and implant sizing device.

FIG. 88 is an illustrative schematic of a medical device with a sensor configured to detect distance between the medical device and a tissue wall.

FIG. 89 is an illustrative schematic of an example delivery system facilitating deployment of an implant with distal advancement of a sheath.

FIG. 90 is an illustrative schematic of an example delivery system facilitating deployment of an implant with distal and proximal advancement of distal and proximal sheath portions, respectively.

### DETAILED DESCRIPTION

As discussed above, existing approaches to treating COPD are either highly invasive (e.g., lung volume reduction surgery), ineffective for most patients (e.g., one-directional stent valves), have an undue impact on gas exchange by healthy lung tissue (e.g., endobronchial coils and clips), carry a high risk of complications (e.g., bronchoscopic thermal vapor ablation), have poor long-term efficacy (e.g., bypass tract prosthetics), and/or suffer from one or more other major limitations. Overcoming these limitations is a significant technical challenge. As discussed in detail below, the inventors have developed new approaches to treating COPD that address at least some of the deficiencies of conventional approaches. In at least some cases, these new approaches are surprisingly effective at establishing and maintaining airway patency. Moreover, this is expected to be the case both in emphysema patients without collateral ventilation and in emphysema patients with collateral ventilation. Approaches to treating COPD in accordance with at least some embodiments of the present technology include the use of innovative endobronchial implants. Aside from the potential clinical benefits, these implants may have better deliverability, retrievability, and/or safety characteristics relative to conventional devices. Given the prevalence and severity of COPD, the innovative endobronchial implants and other aspects of the treatment of COPD in accordance with various embodiments of the present technology has great potential to has a meaningful positive impact on worldwide public health.

At least some embodiments of the present technology are directed to establishing and maintaining patency in obstructed and/or narrowed portions of one or more airways of a lung. This can have a therapeutic benefit for patients diagnosed with COPD, including patients diagnosed with emphysema and/or chronic bronchitis. At least some of this therapeutic benefit may be associated with facilitating the release of air from hyperinflated and/or diseased lung portions along with a corresponding increase in intrathoracic volume available for gas exchange by other lung portions. Implants in accordance with at least some embodiments of the present technology are configured to be intraluminally positioned within an airway and expanded against the airway wall, thereby distending and/or dilating the airway and increasing the cross-sectional area of the airway lumen. In at least some cases, the implants are configured to enlarge the airway beyond its normal size.

In at least some cases, implants in accordance with embodiments of the present technology are configured to have relatively little (e.g., minimal) surface contact with an airway wall and/or to maintain stable contact with an airway wall during respiration. These and other features disclosed herein may reduce or eliminate the gradual airway occlusion by biological processes (e.g., inflammation, fibrosis, granulation, mucous impaction, etc.) that would otherwise limit the effectiveness of implants for the treatment of COPD. An overview of the relevant anatomy and physiology of the lungs as well as additional details regarding implants in accordance with embodiments of the present technology are discussed below.

Many specific details of devices, systems, and methods in accordance with various embodiments of the present technology are disclosed herein. Although these devices, systems, and methods may be disclosed primarily or entirely in the context of treating COPD (sometimes emphysema in particular) other contexts in addition to those disclosed herein are within the scope of the present technology. For example, suitable features of described devices, systems, and methods can be implemented in the context of treating tracheobronchomalacia (TBM) or benign prostatic hyperplasia (BPH) among other examples. Furthermore, it should understood in general that other devices, systems, and methods in addition to those disclosed herein are within the scope of the present technology. For example, devices, systems, and methods in accordance with embodiments of the present technology can have different and/or additional configurations, components, and procedures than those disclosed herein. Moreover, a person of ordinary skill in the art will understand that devices, systems, and methods in accordance with embodiments of the present technology can be without one or more of the configurations, components, and/or procedures disclosed herein without deviating from the present technology.

### I. ANATOMY AND PHYSIOLOGY

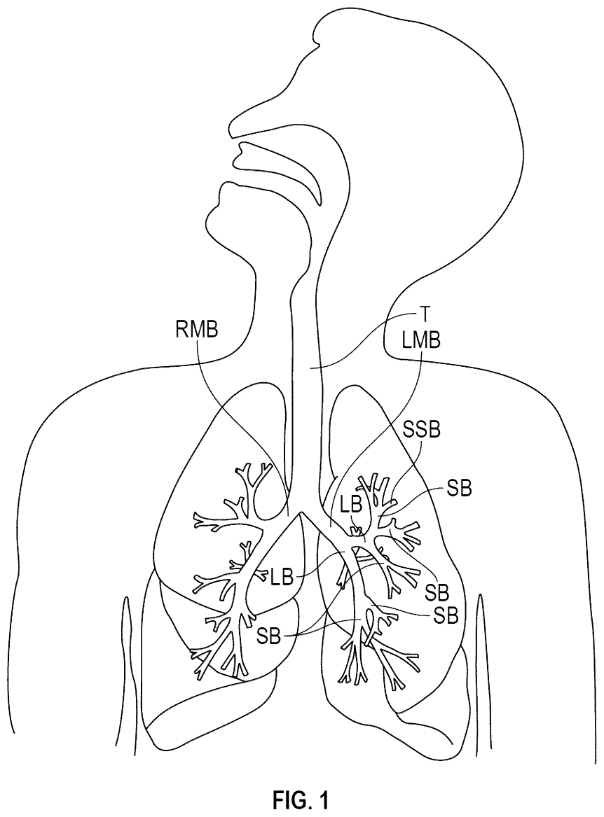

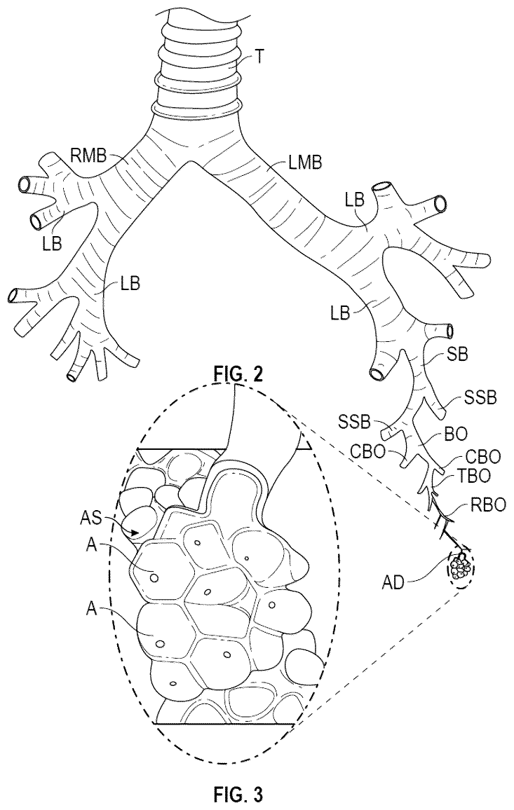

FIG. 1 is a schematic illustration of a bronchial tree of a human subject within a chest cavity of the subject. As shown in FIG. 1, the bronchial tree includes a trachea T that extends downwardly from the nose and mouth and divides into a left main bronchus LMB and a right main bronchus RMB. The left main bronchus and the right main bronchus each branch to form lobar bronchi LB, segmental bronchi SB, and sub-segmental bronchi SSB, which have successively smaller diameters and shorter lengths as they extend distally. FIG. 2 is a schematic illustration of the bronchial tree in isolation. As shown in FIG. 2, the sub-segmental bronchi continue to branch to form bronchioles BO, conducting bronchioles CBO, and finally terminal bronchioles TBO, which are the smallest airways that do not contain alveoli. The terminal bronchioles branch into respiratory bronchioles RBO, which divide into alveolar ducts AD. FIG. 3 is an enlarged view of a terminal portion of the bronchial tree. As shown in FIG. 3, the alveolar ducts terminate in a blind outpouching including two or more small clusters of alveoli A called alveolar sacs AS. Various singular alveoli can be disposed along the length of a respiratory bronchiole as well.

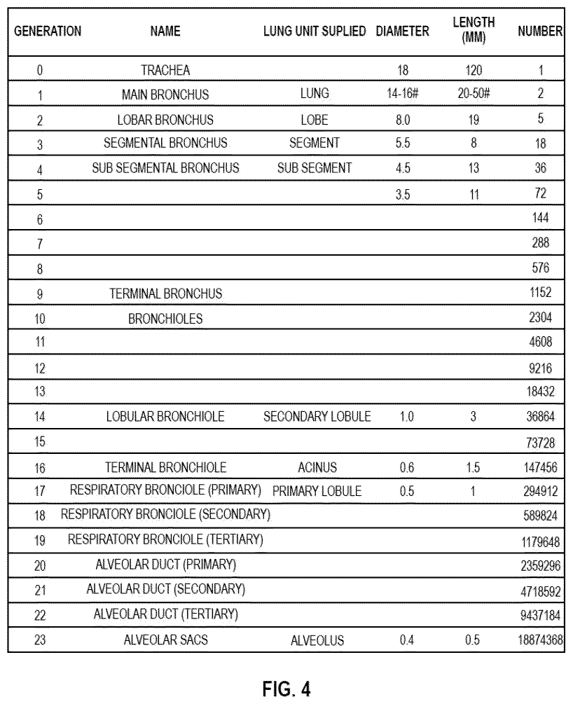

Bronchi and bronchioles are conducting airways that convey air to and from the alveoli. They do not take part in gas exchange. Rather, gas exchange takes place in the alveoli that are found distal to the conducting airways, starting at the respiratory bronchioles. It is common to refer to the various airways of the bronchial tree as “generations” depending on the extent of branching proximal to the airways. For example, the trachea is referred to as “generation 0” of the bronchial tree, various levels of bronchi, including the left and right main bronchi, are referred to as “generation 1,” the lobar bronchi are referred to as “generation 2,” and the segmental bronchi are referred to as “generation 3.” Further, it is common to refer to any of the airways extending from the trachea to the terminal bronchioles as “conducting airways.” FIG. 4 is a table indicating examples of dimensions and generation numbers of different portions of the bronchial tree.

The respiratory bronchioles, alveoli, and alveolar sacs receive air via more proximal portions of the bronchial tree and participate in gas exchange to oxygenate blood routed to the lungs from the heart via the pulmonary artery, branching blood vessels, and capillaries. Thin, semi-permeable membranes separate oxygen-depleted blood in the capillaries from oxygen-rich air in the alveoli. The capillaries wrap around and extend between the alveoli. Oxygen from the air diffuses through the membranes into the blood. Carbon dioxide from the blood diffuses through the membranes to the air in the alveoli. The newly oxygen-enriched blood then flows from the alveolar capillaries through the branching blood vessels of the pulmonary venous system to the heart. The heart pumps the oxygen-rich blood throughout the body. The oxygen-depleted air in the lungs is exhaled when the diaphragm and intercostal muscles relax and the lungs and chest wall elastically return to their normal relaxed states. In this manner, air flows through the branching bronchioles, segmental bronchi, lobar bronchi, main bronchi, and trachea, and is ultimately expelled through the mouth and nose.

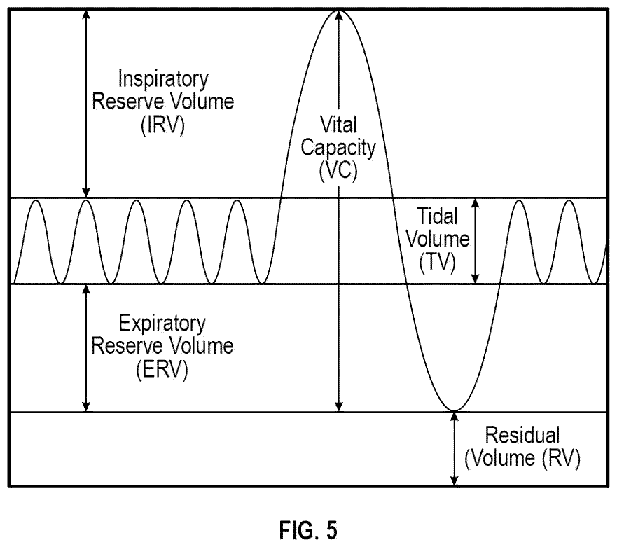

FIG. 5 is a diagram showing lung volumes during normal lung function. Approximately one-tenth of the total lung capacity is used at rest. Greater amounts are used as needed (e.g., with exercise). Tidal Volume (TV) is the volume of air breathed in and out without conscious effort. The additional volume of air that can be exhaled with maximum effort after a normal inspiration is Inspiratory Reserve Volume (IRV). The additional volume of air that can be forcibly exhaled after normal exhalation is Expiratory Reserve Volume (ERV). The total volume of air that can be exhaled after a maximum inhalation is Vital Capacity (VC). VC equals the sum of the TV, IRV, and ERV. Residual Volume (RV) is the volume of air remaining in the lungs after maximum exhalation. The lungs can never be completely emptied. The Total Lung Capacity (TLC) is the sum of the VC and RV. Evaluation of lung function may be used to determine a patient's eligibility for therapy, as well as to evaluate a therapy's effectiveness.

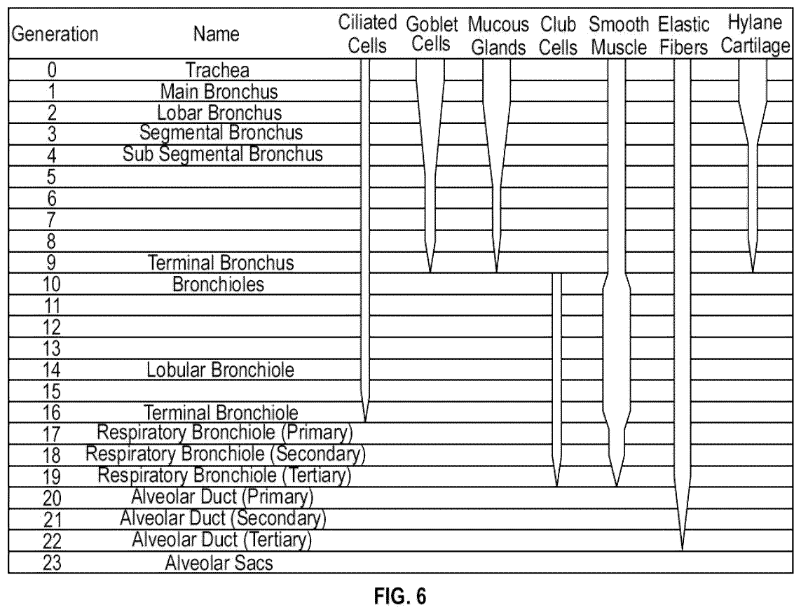

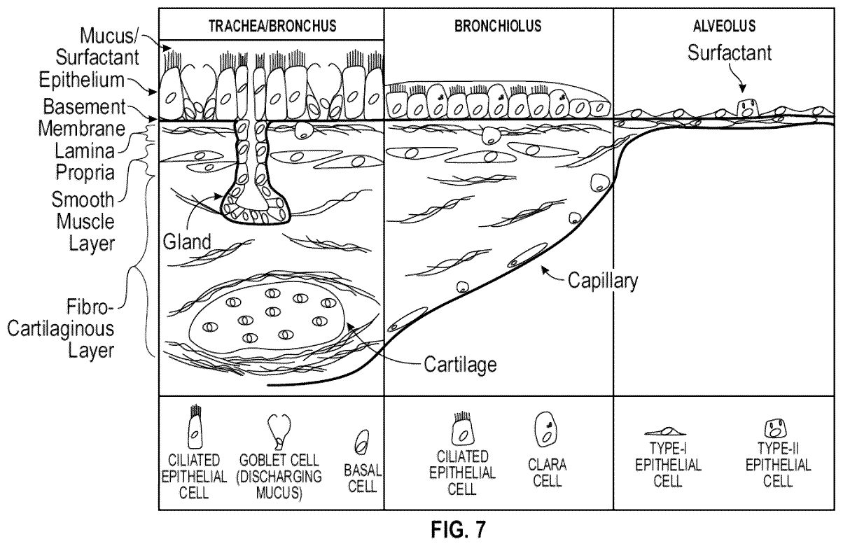

FIG. 6 is a table showing airway wall composition at different portions of a bronchial tree. FIG. 7 is an anatomical illustration of airway wall composition at different portions of a bronchial tree. As shown in FIGS. 6 and 7, the walls of the bronchi, bronchioles, alveolar ducts and alveoli are include epithelium, connective tissue, goblet cells, mucous glands, club cells, smooth muscle elastic fibers, and hyaline cartilage with nerves, blood vessels, and inflammatory cells interspersed throughout. Most of the epithelium (from the nose to the bronchi) is covered in ciliated pseudostratified columnar epithelium, commonly called respiratory epithelium. The cilia located on these epithelium beat in one direction, moving mucous and foreign material such as dust and bacteria from the more distal airways to the more proximal airways and eventually to the throat, where the mucus and/or foreign material are cleared by swallowing or expectoration. Moving down the bronchioles, the cells are more cuboidal in shape but are still ciliated.

The proportions and properties of various components of the airway wall vary depending on the location within the bronchial tree. For example, mucous glands are abundant in the trachea and main bronchi but are absent starting at the bronchioles (e.g., at approximately generation 10). In the trachea, cartilage presents as C-shaped rings of hyaline cartilage, whereas in the bronchi the cartilage takes the form of interspersed plates. As branching continues through the bronchial tree, the amount of hyaline cartilage in the walls decreases until it is absent in the bronchioles. Smooth muscle starts in the trachea, where it joins the C-shaped rings of cartilage. It continues down the bronchi and bronchioles, which it completely encircles. Instead of hard cartilage, the bronchi and bronchioles are composed of elastic tissue. As the cartilage decreases, the amount of smooth muscle increases. The mucous membrane also undergoes a transition from ciliated pseudostratified columnar epithelium to simple cuboidal epithelium to simple squamous epithelium.

### II. PULMONARY DISEASE

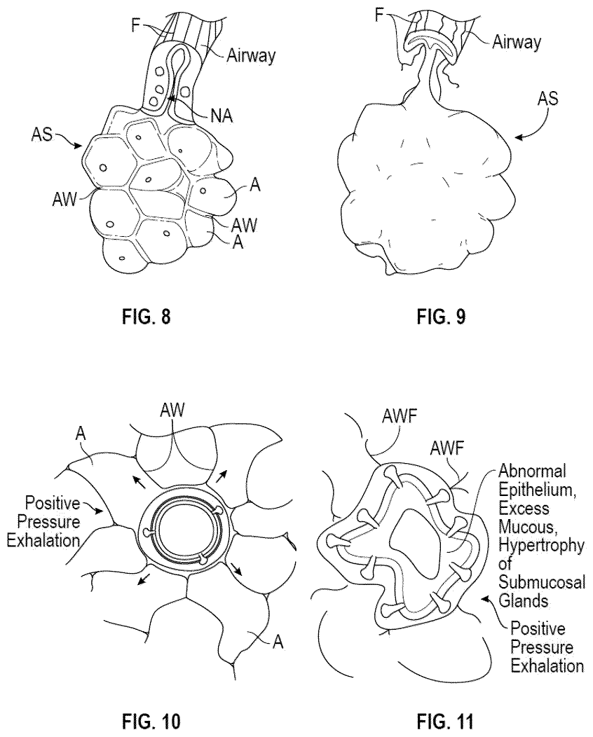

FIG. 8 is an anatomical illustration showing small airway narrowing in emphysematous lung tissue. FIG. 9 is an anatomical illustration showing alveolar wall damage in emphysematous lung tissue. FIG. 10 is an anatomical illustration showing normal airway patency during exhalation. FIG. 11 is an anatomical illustration showing airway collapse during exhalation in emphysematous lung tissue. COPD, and emphysema in particular, is characterized by irreversible destruction of the alveolar walls that contain elastic fibers that maintain radial outward traction on small airways and are useful in inhalation and exhalation. As shown in FIGS. 8-11, when these elastic fibers are damaged, the small airways are no longer under radial outward traction and collapse, particularly during exhalation. Furthermore, emphysema destroys the alveolar walls. As shown in FIG. 9, this results in one larger air space and reduces the surface area available for gas exchange. The lungs are thus unable to perform gas exchange at a satisfactory rate, which causes a reduction in oxygenated blood. Additionally, the large air spaces of diseased lung combined with collapsed airways results in hyperinflation (air trapping) of the lung and an inability to fully exhale. Moreover, the hyperinflated lungs apply continuous pressure on the chest wall, diaphragm, and surrounding structures, which causes shortness of breath and can prevent a patient from walking short distances or performing routine tasks. Both quality of life and life expectancy for patients with late-stage emphysema are extremely low, with fewer than half of patients surviving an additional five years.

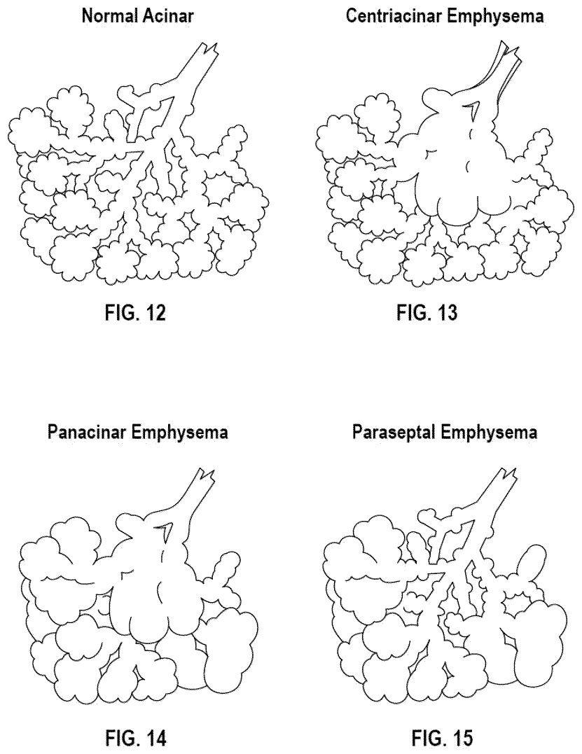

There are three types of emphysema: centriacinar, panacinar, and paraseptal. FIG. 12 is an anatomical illustration showing normal acinar. FIG. 13 is an anatomical illustration showing centriacinar emphysema, which involves the alveoli and airways in the central acinus, including destruction of the alveoli in the walls of the respiratory bronchioles and alveolar ducts. FIG. 14 is an anatomical illustration showing panacinar emphysema, which is characterized by destruction of the tissues of the alveoli, alveolar ducts, and respiratory bronchioles. This produces a fairly uniform dilatation of the air space throughout the acini and evenly distributed emphysematous changes across the acini and the secondary lobules. FIG. 15 is an anatomical illustration showing paraseptal emphysema, which is characterized by enlarged airspaces at the periphery of acini resulting predominately from destruction of the alveoli and alveolar ducts. The distribution of the paraseptal emphysema is usually limited in extent and occurs most commonly along the posterior surface of the upper lung. It often coexists with other forms of emphysema.

One further aspect of the progression of emphysema and associated alveolar wall destruction is that the airflow between neighboring alveoli, known as collateral ventilation or collateral air flow, is increased. Collateral ventilation can significantly undermine the clinical utility of endobronchial valves. As discussed above, these valves are designed to allow one-way air passage to cause atelectasis of the diseased lobe. However, collateral ventilation causes inflation of the lobe, thereby preventing atelectasis.

### III. ENDOBRONCHIAL IMPLANTS

Described herein are devices, technologies, and methods for treating patients having pulmonary disease, such as severe emphysema. At least some embodiments of the present technology include endobronchial placement of an implant to establish or improve airway patency. The implant can be placed at a treatment location including a previously collapsed airway, such as a previously collapsed distal airway. Deployment of the implant can release air trapped in a hyperinflated portion of the lung and/or reduce or prevent subsequent trapping of air in this portion of the lung. In at least some cases, it is desirable for a treatment location at which an implant is deployed to include an airway of generation 4 or higher/deeper, such as (from distal to proximal) the respiratory bronchioles, terminal bronchioles, conducting bronchioles, bronchioles or sub-segmental bronchi and then run proximally to a more central, larger airway (e.g., 6th generation or more proximal/lower) such as (from distal to proximal) sub-segmental bronchi, segmental bronchi, lobar bronchi and main bronchi. A single implant may create a contiguous path distal to proximal to reliably create passage for the trapped air. In an alternative embodiment, multiple, discrete implants can be used instead of a single, longer implant. The multiple, discrete implants may be placed in bronchial airways that have collapsed or are at risk of collapse. The use of multiple, discrete implants in select locations in the bronchial tree may have the advantage of using less material, thereby reducing contact stresses and foreign body response (discussed supra), and allow for greater flexibility and customization of therapy. For example, whereas a single implant embodiment may run from a higher generation airway distally to a lower generation airway proximally, a system of multiple, discrete implants may allow for placement of implants in multiple airways of the same generation.

The devices, systems and methods described herein may be administered to different bronchopulmonary segments in order to release trapped air from regions of the lung in the safest and most efficient manner possible. For example, treatment of the left lung may involve one or more of the following segments: Upper Lobe (Superior: apical-posterior, anterior; Lingular: superior, inferior); Lower Lobe: superior, antero-medial basal, lateral basal. Treatment of the right lung may involve one or more of the following segments: Upper Lobe: apical, anterior, posterior; Middle Lobe: medial, lateral; Lower Lobe: superior, anterior basal, lateral basal. The treatments described herein may involve placement of a single implant in a single lung (right or left), a single implant in each lung or multiple implants in each lung. Treatment within a particular lung may involve placing an implant in a specific lobe (e.g., upper lobe) and a specific segment within such lobe or it may involve placement of at least one implant in multiple lobes, segments within a lobe or sub-segments within a segment. Determination of which parts of the lung to treat can be made by the clinical operator (e.g., pulmonologist or surgeon) with the assistance of imaging (e.g., CT, ultrasound, radiography, or bronchoscopy) to assess the presence and pathology of disease and impact on pulmonary function and airflow dynamics.

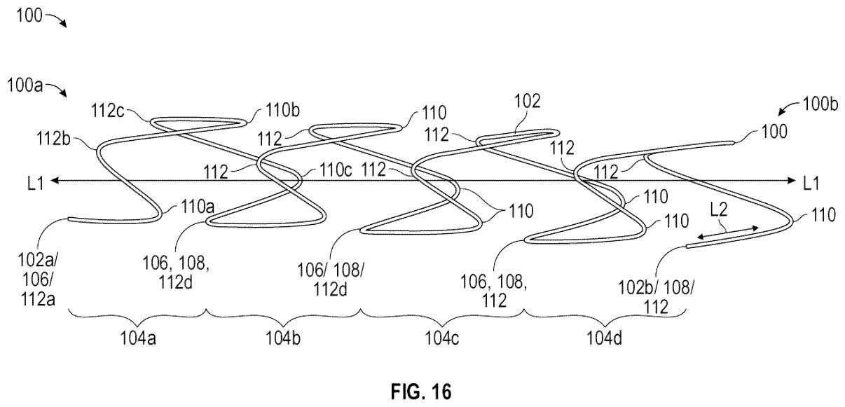

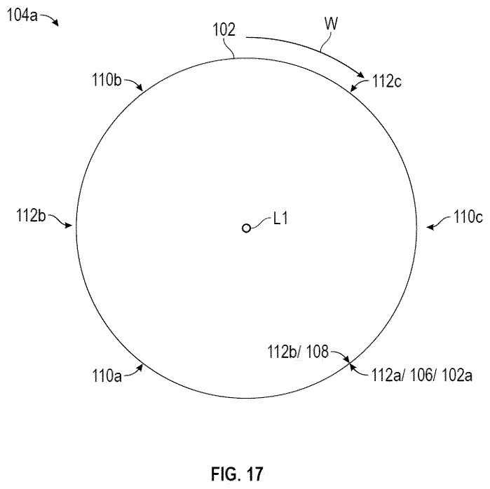

FIG. 16 is a side view of an expandable device 100 configured to be positioned in an airway lumen, shown in an expanded, unconstrained state. FIG. 17 is an end view of the device 100. As shown in FIG. 16, the device 100 can comprise a generally tubular structure configured to be positioned within an airway lumen. For example, the device 100 may be configured to be implanted in an airway lumen such that the device 100 maintains a lumen of a minimum desired diameter in the airway. The device 100 has a first end portion 100a, a second end portion 100b opposite the first end portion 100a, and a central longitudinal axis L1 extending between the first and second end portions 100a, 100b. As used herein, the term “longitudinal” can refer to a direction along an axis that extends through the lumen of the device while in a tubular configuration, the term “circumferential” can refer to a direction along an axis that is orthogonal to the longitudinal axis and extends around the circumference of the device when in a tubular configuration, and the term “radial” can refer to a direction along an axis that is orthogonal to the longitudinal axis and extends toward or away from the longitudinal axis.

The device 100 can comprise an elongated member 102 wound about the longitudinal axis L1 of the device 100. In some embodiments, the elongated member 102 is heat set in a novel three-dimensional (3D) configuration such that the elongated member 102 is configured to self-expand to the preset configuration. In some embodiments, the elongated member 102 is not heat set and/or configured to self-expand. For example, the elongated member 102 can be balloon-expandable. In some embodiments, the elongated member 102 is balloon-expandable and self-expanding. The elongated member 102 has a first end 102a and a second end 102b opposite the first end 102a along a longitudinal axis L2 of the elongated member 102. The elongated member 102 can comprise a wire, a coil, a tube, a filament, a single interwoven filament, a plurality of braided filaments, a laser-cut sheet, a laser-cut tube, a thin film formed via a deposition process, and/or other suitable elongated structures and/or methods, such as cold working, bending, EDM, chemical etching, water jet, etc. The elongated member 102 can be formed using materials such as nitinol, stainless steel, cobalt-chromium alloys (e.g., 35N LT®, MP35N (Fort Wayne Metals, Fort Wayne, Indiana)), Elgiloy, magnesium alloys, tungsten, tantalum, platinum, rhodium, palladium, gold, silver, or combinations thereof, or one or more polymers, or combinations of polymers and metals. In some embodiments, the elongated member 102 may include one or more drawn-filled tube (“DFT”) wires comprising an inner material surrounded by a different outer material. The inner material, for example, may be radiopaque material, and the outer material may be a superelastic material.

Although the device 100 shown in FIG. 16 comprises a single elongated member 102, the device 100 may comprise any number of elongated members 102. A single elongated member, such as a single wire expandable device, can be easier to remove and/or reposition as the operator can grab the elongated member on one end and pull it through a working channel of a scope. The elongated member will straighten out in either the balloon expandable or self-expanding form.

Referring to FIG. 16, the elongated member 102 may be wound about the longitudinal axis L1 of the device 100 in a series of windings or loops 104, four of which are shown in FIG. 16 and individually labeled 104a-104d. Each of the loops 104 can extend around the longitudinal axis L1 of the device 100 between a first end 106 and a second end 108. In some embodiments, the loops 104 are connected end to end such that, for example, a second end 108 of the first loop 104a is the first end 106 of the second loop 104b. The second end 108 can be disposed approximately 360 degrees from the first end 106 about the longitudinal axis L1 of the device 100. That is, the first and second ends 106, 108 can be disposed at generally equivalent circumferential positions relative to the longitudinal axis L1 of device 100. In some embodiments, the device 100 has a circular cross-sectional shape. In other embodiments, the device 100 may have other suitable cross-sectional shapes (e.g., oval, square, triangular, polygonal, irregular, etc.). The cross-sectional shape of the device 100 may be generally the same or vary along the length of the device 100 and/or from loop to loop.

The expanded cross-sectional dimension of the device 100 may be generally constant or vary along the length of the device 100 and/or from loop to loop. For example, as discussed herein, the device 100 can have varying cross-sectional dimensions along its length to accommodate different portions of the airway. For instance, the device 100 can have a first cross-sectional dimension along a first portion configured to be positioned in a more distal portion of the airway (such as, for example, in a terminal bronchiole and/or emphysematous areas of destroyed and/or collapsed airways), and a second cross-sectional dimension along a second portion configured to be positioned more proximally (such as in a primary bronchus and/or another portion that has not collapsed). The second portion, for example, can be configured to be positioned in a portion of the airway that is less emphysematous than the collapsed distal portion and/or has cartilage in the airway wall (preferably rings of cartilage and not plates), which can occur at the lobar (generation 2) or segmental (generation 3) level.

In some embodiments, the expanded cross-sectional dimension of the device 100 in an unconstrained (i.e., removed from the constraints of a catheter or airway), expanded state is oversized relative to the diameter of the native airway lumen. For example, the expanded, unconstrained cross-sectional dimension of the device 100 can be at least 1.5× the original (non-collapsed) diameter of the airway lumen in which it is intended to be positioned. In some embodiments, the device 100 has an expanded, cross-sectional dimension that is about 1.5× to 6×, 2× to 5×, or 2× to 3× the diameter of the original airway lumen. Without being bound by theory, it is believed that expanding the airway lumen to the greatest diameter possible without tearing the airway wall will provide the greatest improvement in pulmonary function (for example, as measured by outflow, FEV, and others).

As shown in FIG. 16, the elongated member 102 may undulate along its longitudinal axis L2 as it winds around the longitudinal axis L1 of the device 100, forming a plurality of alternating peaks 110 (closer to the second end portion 100b of the device 100) and valleys 112 (closer to the first end portion 100a of the device 100). At least some of the valleys 112 can be at different locations along the longitudinal axis L1 of the device 100 than at least some of the peaks 110. Additionally or alternatively, at least some of the valleys 112 can be at different longitudinal locations than at least some others of the valleys 112 and/or at least some of the peaks 110 can be at different longitudinal locations than at least some others of the peaks 110.

As an example, three peaks 110 and four valleys 112 of the first loop 104a have been individually labeled as peaks 110a-110c and valleys 112a-d. As shown in FIGS. 16 and 17, for the first loop 104a in the direction of the wind W, the elongated member 102 extends from the first end 106 of the elongated member 102, which comprises a first valley 112a of the first loop 104a, to a first peak 110a of the first loop 104a along a first longitudinal direction toward the second end portion 100b of the device 100. The elongated member 102 can then extend from the first peak 110a to a second valley 112b along a second longitudinal direction opposite of the first longitudinal direction, from the second valley 112b to a second peak 110b along the first longitudinal direction, from the second peak 110b to a third valley 112c along the second longitudinal direction, from the third valley 112c to a third peak 110c along the first longitudinal direction, and from the third peak 110c to a fourth valley 112d (which is also the second end 108 of the first loop 104a) along the second longitudinal direction. Thus, when traveling in a direction of the wind W around a given loop 104, the loop 104 does not consistently progress from the first end portion 100a of the device 100 to the second end portion 100b of the device 100 (or vice versa), but rather undulates so that along certain portions of its length, the loop 104 becomes progressively closer to the first end portion 100a of the device 100, and along other portions of its length the loop becomes progressively closer to the second end portion 100b of the device 100.

Although the first and second ends 106, 108 of one of the loops 104 may be generally aligned circumferentially, the first and second ends 106, 108 are longitudinally offset. The first peak 110a can be closer to the second end portion 100b of the device 100 than the first valley 112a. The second valley 112b can be closer to the first end portion 100a of the device 100 than the first peak 110a and/or the first valley 112a. The second peak 110b can be closer to the second end portion 100b of the device 100 than the second valley 112b, the first peak 110a, and/or the first valley 112a. The third valley 112c can be closer to the first end portion 100a of the device 100 than the second peak 110b and/or closer to the second end portion 100b of the device 100 than the first valley 112a and/or the second valley 112b. In some embodiments, the third valley 112c can be substantially longitudinally aligned with the first peak 110a. The third peak 110c can be closer to the second end portion 100b of the device 100 than the third valley 112c, the second peak 110b, the second valley 112b, the first peak 110a, and/or the first valley 112a. The fourth valley 112d can be closer to the first end portion 100a of the device 100 than the third peak 110c and/or closer to the second end portion 100b of the device 100 than the third valley 112c, the second valley 112b, the first peak 110a, and/or the first valley 112a. In some embodiments, the fourth valley 112d can be substantially longitudinally aligned with the second peak 110b.

Although FIGS. 16 and 17 show a device 100 comprising four loops 104, each having four peaks 110 and four valleys 112, in some embodiments one or more of the loops 104 has more or fewer peaks 110 and/or more or fewer valleys 112. For example, in some embodiments one or more of the loops 104 has one, two, three, four, five, six, seven, eight, etc. peaks 110 per loop 104 and one, two, three, four, five, six, seven, eight, etc. valleys 112 per loop 104. The loops 104 may have the same or a different number of peaks 110, and the loops 104 may have the same or a different number of valleys 112. A circumferential distance (e.g., an angular separation) between adjacent ones of the peaks 110 and valleys 112 can be uniform or non-uniform in a given loop 104. In some embodiments, adjacent ones of the peaks 110 and valleys 112 can be spaced apart around a circumference of the device 100 by about 90 degrees, about 120 degrees, about 150 degrees, about 180 degrees, about 210 degrees, about 240 degrees, about 270 degrees, about 300 degrees, and/or about 330 degrees. In addition, the amplitude of the peaks 110 may be the same or different along a given loop 104 and/or amongst the loops 104, and the amplitude of the valleys 112 may be the same or different along a given loop 104 and/or amongst the loops 104. Moreover, the peaks 110 and valleys 112 can have the same or different amplitudes.

As shown in FIG. 16, a portion of the elongated member 102 between adjacent peaks 110 and valleys 112 can be linear, curved, or both. Adjacent portions of the elongated member 102 between two sets of adjacent peaks 110 and valleys 112 can form a V-shaped and/or U-shaped structure. At least some of the valleys 112 can be concave toward the second end portion 100b of the device 100 and/or at least some of the peaks 110 can be concave toward the first end portion 100a of the device 100.

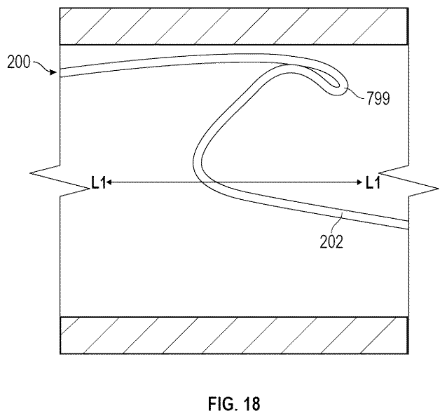

In some embodiments, for example as shown in FIG. 16, the elongated member 102 can extend around a circumference of the device 100 and/or along a longitudinal axis L1 of the device 100, without substantially extending radially away or towards the longitudinal axis L1. Still, in some embodiments, a device 200 can comprise an elongated member 202 that undulates radially with respect to a longitudinal axis L1 of the device 200. As shown in FIG. 18, for example, the elongated member 202 can form peaks 799 and/or valleys 799 that are located closer to the longitudinal axis L1 than intermediate portions of the elongated member 202 between the peaks 799 and valleys 799. The apices of each “V” can be bent radially inward toward the center of the lumen, so that only the longitudinally-extending portions of the elongated member 202 are touching the bronchial wall. Such a configuration can prevent the stent from impeding mucus flow along the wall of the bronchus.

The radial mechanism of expansion allows the expandable device to be easily designed and delivered by both self-expansion and balloon-expansion. The zig-zag pattern of the devices disclosed herein, including the example shown in FIG. 16, is configured to conform to different diameter airways with a single design, whereas conventional coils are a fixed diameter. This is especially advantageous for achieving gradual airway dilation over time. The expandable device stores expansion potential in the stent design which is achieved via beams that bend and elastic potential is established. The expandable device in balloon expandable form also has a unique potential to form a coil by expanding the zig-zags all the way to a straight line when geometrically designed this way.

An expandable device can be configured to be positioned within a lumen of an airway such that the expandable device increases a diameter of the lumen and thereby facilitates and/or improves transport of gas through the airway. In some embodiments, an expandable device can be positioned within an airway lumen that is collapsed, narrowed, or otherwise reduced in diameter. Expandable devices of the present technology can have a radial resistive force (RRF) that resists compression of the expandable device by the airway wall and/or a chronic outward force (COF) that is applied to the airway wall by the expandable device. The RRF and/or the COF of an expandable device can be of a significant magnitude such that the expandable device is configured to maintain a minimum desired diameter of the airway lumen. An expandable device of the present technology and/or one or more portions thereof can comprise a stent, a braid, a mesh, a weave, a fabric, a coil, a tube, a valve, and/or another suitable device configured to be positioned within an anatomical passageway, airway lumen or vessel to provide support to the passageway and/or another medical device, and/or to modify biological tissue of the passageway.

In certain other applications, it may be desirable for an expandable device to be configured to contact a large surface area of a wall of a passageway. For example, coronary stents are often designed such the stent is configured to contact a large surface area of a wall of a patient's coronary artery. Such design may be advantageous for expandable devices configured to be positioned within a blood vessel in order to prevent or limit adverse outcomes (e.g., expandable device thrombosis, neoatherosclerosis, etc.) associated with interactions between the expandable device and the patient's blood. However, because an airway is configured to transport air, not blood, there is no risk of clotting in the airways. Moreover, while clotting is not a risk in the airways, excessive granulation tissue can form in the airways due to contact and/or relative motion between an expandable device and the airway wall. Such excessive granulation tissue can narrow the airway lumen and inhibit gas transport through the airway. Thus, it may be advantageous for an expandable device configured to be positioned within an airway to be configured to contact a smaller surface area of an airway lumen to prevent or limit granulation tissue formation, facilitate mucous clearance from the airway, etc.

It should be appreciated that the goal of the expandable device is not to eliminate the formation of granulation tissue, as some formation of granulation tissue is expected with any foreign body in the airway, but rather to minimize any clinically meaningful obstruction caused by granulation tissue and/or mucus. It is anticipated that an expandable device with significantly lower contact area will experience a focal foreign body response (FBR) that will not cause obstruction of the primary airway or distal airways. A certain amount of focal response might actually be of benefit as partial or full encapsulation of the expandable device may provide stronger mechanical reinforcement of the airway lumen and/or help anchor the expandable device to resist movement due to breathing or coughing.

COF can also help prevent migration of the implanted expandable device in a patient's airways. However, excessive COF may result in elevated mechanical stress at the implant-tissue interface, which can in some instances trigger a severe FBR. This may lead to occlusion of the expandable device and failure. Thus, a desired COF parameter for an expandable device can be determined based on careful consideration to balance risks (e.g., FBR) and benefits (e.g., airway dilation).

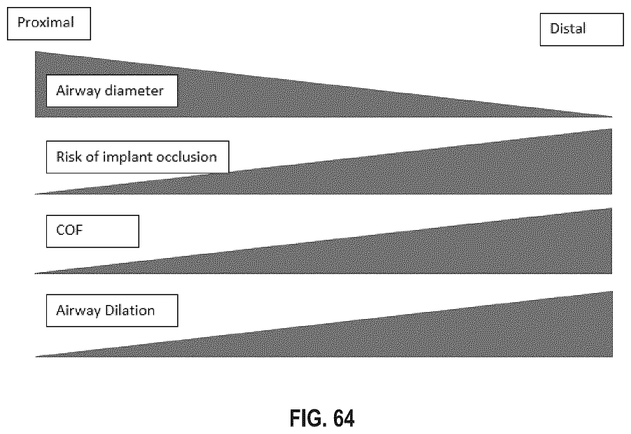

As shown in FIG. 64, it is contemplated that the risk of airway and expandable device occlusion due to a foreign body response to implantation of the expandable device is generally greater at a distal end of the expandable device in contact with smaller distal airways, compared to at a proximal end of the expandable device in contact with larger proximal airways, because even moderate levels of foreign body response can obstruct small airways. Therefore, it is anticipated that a higher COF resulting in increased dilation is more beneficial for the distal airways. Additionally, due to native tissue damage (e.g., emphysema-related tissue damage), the increase in FBR in a patient due to high COF may be less pronounced in the distal airways. Accordingly, in some embodiments, an expandable device design featuring a gradual increase in COF from the proximal end to the distal end may optimize implant functionality while reducing the risk of FBR occlusion. Although FIG. 64 schematically illustrates this general trend as a linear relationship, it should be understood that the desired rate of increase in COF from the proximal end to the distal end may not necessarily be linear and may depend at least in part on, for example, anatomical and/or tissue characteristics, such as variations in airway dimension, mechanical tissue properties, etc. Similarly, the risk of expandable device occlusion and/or desired airway dilation may not necessarily follow a linear relationship with airway diameter as shown in FIG. 64.

In some embodiments, for example, the expandable device can include a variable COF along its length. For example, an expandable device can include a first proximal implant portion and a second distal implant portion that is more distal than the first implant portion, where the second distal implant portion is configured to provide a greater COF than the first proximal implant portion. Furthermore, the expandable device can include an intermediate portion between the first proximal implant portion and the second distal implant portion, where the intermediate portion is configured to exert a variable COF along its length (e.g., ranging between the first and second COFs).

The variable COF can, for example, range between the COF exerted by the first proximal implant portion and the COF exerted by the second distal implant portion. In some embodiments, the second COF at the distal end can be between about 1.1 times and about 5 times larger than the first COF at the proximal end. In some embodiments, the second COF at the distal end can be between about 2 times and about 4 times larger than the first COF at the proximal end. For example, the COF at the distal end can be about 2 times, about 2.2 times, about 2.5 times, about 2.8, about 3 times, about 3.2 times, or about 3.5 times, or about 3.8 times larger than the first COF at the proximal end. In some embodiments, for example, a distal portion of the expandable device can exert a COF of between about 0.20 N/mm (normalized over stent length) and about 0.35 N/mm, while a proximal portion of the expandable device can exert a COF of between about 0.08 N/mm and about 0.14 N/mm. In one specific example, a distal portion of the expandable device can exert a COF of about 0.32 N/mm and a proximal portion of the expandable device can exert a COF of about 0.08 N/mm.

It should be understood that in some embodiments, the radial resistive force of the expandable device may also vary along its length for improving airway function. In other words, in some embodiments, the expandable device can include a variable RRF along its length. For example, an expandable device can include a first proximal implant portion and a second distal implant portion that is more distal than the first implant portion, where the second distal implant portion is configured to provide a greater RRF than the first proximal implant portion. Furthermore, the expandable device can include an intermediate portion between the first proximal implant portion and the second distal implant portion, where the intermediate portion is configured to exert a variable RRF along its length (e.g., ranging between the first and second RRFs).

In some embodiments, the COF and/or RRF for different portions (e.g., proximal portion, intermediate portion, distal portion, etc.) of the expandable device can be configured as the result of any one or more various geometrical features of the expandable device. For example, the diameter of the wire forming the expandable device can vary along the device length (e.g., wire can increase in diameter from the device's proximal end to the distal end). As another example, the diameter of the expandable device can vary along the device length (e.g., the device diameter can increase from the device's proximal end to the distal end), as when the wire is heat set, segments of different wire diameters can have different resulting material characteristics. As another example, the radii of curvature of wire bends can vary along the device length (e.g., radius of curvature in any one or more of the zig-zags or peaks/valleys of the device can decrease from the device's proximal end to the distal end, to increase the spring force exerted by the device). As another example, a distal portion of the expandable device can include an additional zig-zag repeating pattern (e.g., four repeats instead of three). As another example, the heat treatment along the length of the implant can vary to tune different strengths along the length of the implant. As yet another example, a distal portion of the expandable device can include more turns of the wire to increase the spring force exerted by the device. Any one or more of these approaches can be combined to configure a device with variable COF and/or RRF along its length. However, implementation of these device features should be carefully considered against factors such as reducing foreign body response due to the surface area contact between the device and the surrounding airway lumen, reducing the risk of introducing excessive strain on the device when crimping the expandable device into a radially compressed configuration (e.g., for loading onto a delivery device).

The minimum desired diameter of the airway lumen can be based on a desired capacity for air flow through the airway. In some embodiments, the minimum desired diameter of the airway lumen is based, at least in part, on a nominal diameter of a lumen of a corresponding airway in healthy patients. In some embodiments, the nominal diameter is based on measurements obtained from healthy patients of similar demographics (e.g., sex, age, race, etc.). Additionally or alternatively, the minimum desired diameter of the airway lumen can be based, at least in part, on a diameter of one or more airway lumens in a specific patient. In some embodiments, the minimum desired diameter of the airway is at least as large as a diameter of a lumen of a healthy airway of a corresponding generation. The minimum desired diameter of the airway lumen can be about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, or about 25 mm. In some embodiments, the minimum desired diameter of the airway is at least 0.1 mm, at least 0.2 mm, at least 0.3 mm, at least 0.4 mm, at least at least 0.5 mm, at least 0.6 mm, at least 0.7 mm, at least 0.8 mm, at least 0.9 mm, at least 1 mm, at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, at least 20 mm, at least 21 mm, at least 22 mm, at least 23 mm, at least 24 mm, or at least 25 mm.

As airflow resistance through an airway is related to the inverse of the fourth power of the radius of the airway lumen, even small increases in the diameter of the airway lumen can significantly improve an airway's airflow capacity. Moreover, as a patient may have multiple collapsed airways that have extremely high airflow resistance, it may be advantageous for an airway treated with an expandable device of the present technology to have an airflow capacity sufficient to compensate for multiple nonfunctioning airways. Thus, it may be advantageous for the expandable device to be configured to maintain a diameter of an airway lumen that is greater than a nominal diameter of a lumen of a corresponding healthy airway.

In some embodiments, the minimum desired diameter of the airway can be based, at least in part, on a desired functional measure and/or outcome measure and/or a desired change in a functional or outcome measure. Such functional and outcome measures can include, but are not limited to, forced vital capacity (FVC), forced expiratory volume in one second (FEV₁), forced expiratory volume in six seconds (FEV₆), functional residual capacity (FRC), total lung capacity (TLC), residual volume (RV), diffusing capacity of the lung for carbon monoxide (D_{L,CO}), (P_{a,O2}), arterial oxygen saturation (S_{p,O2}), health related quality of life (HRQoL), other relevant functional and/or outcome measures, or combinations thereof. For example, it may be acceptable for the minimum desired diameter of the airway lumen to be less than the nominal diameter of a corresponding healthy airway lumen if the minimum desired diameter is associated with desirable and/or sufficient improvements in a functional measure and/or an outcome measure.

In some embodiments, it may be beneficial to perform airflow diagnostic measurements within the airway before, during and/or after administration of the expandable device to confirm improvement in expiratory flow and pulmonary function. Conventional pulmonary function tests like spirometry can be administered separately from the procedure to administer the expandable device or interventional diagnostics can be administered periprocedurally to measure bronchial air flow and pressure (e.g., Chartis® Pulmonary Assessment System). The data obtained from these tests can help inform decisions related to initial treatment, the adequacy of the administered treatment and, if further treatment is required, the extent and location of additional expandable devices.

It may be beneficial for one or more parameters of an expandable device to be based, at least in part, on a property of the airway the device is configured to be positioned within. For example, it may be desirable for a stiffness of an expandable device to correspond to a stiffness of the airway to prevent or limit granulation tissue formation due to relative motion between the device and the airway. Moreover, in order to facilitate transport of air trapped in hyperinflated parenchymal tissue of a patient out of the patient's body via airways, it may be advantageous for an expandable device of the present technology to be configured to span multiple airway generations when the expandable device is implanted. However, this presents several technical challenges as the mechanical and biological properties of the respiratory system are variable from the proximal, extraparenchymal airways (e.g., the trachea, the primary bronchi, etc.) to the distal, intraparenchymal airways (e.g., the bronchioles, etc.). For example, while the walls of the proximal airways contain cartilage and are internally supported, the amount of hyaline cartilage in the airway walls decreases proximally to distally. As a result, the distal, intraparenchymal airways are highly compliant and expansion and contraction of these airways are controlled by alveolar attachments tethered to the airways. To accomplish the above-noted design objectives and overcome the above-noted challenges, an expandable device configured in accordance with several embodiments of the present technology can have one or more parameters that vary along a length of the expandable device.

An expandable device can have at least one region having a stiffness based, at least in part, on mechanical properties of a portion of a patient's airways. For example, because the distal airways are more compliant than the upper airways, a low COF and/or RRF may be sufficient to maintain a desired minimum diameter of a distal airway lumen. Additionally or alternatively, it may be advantageous for a stiffness of a region of an expandable device to be at least partially based on an airway stiffness to prevent or limit granulation tissue forming friction between the device and the airway. For example, because the airways typically decrease in stiffness from the proximal to distal airways, it may be advantageous for an expandable device to also have a decreasing stiffness along its length. In some embodiments, a distal end of an expandable device that is configured to be positioned within intraparenchymal airways can have a lower stiffness than a proximal end of the expandable device that is configured to be positioned within extraparenchymal airways. In some embodiments, a proximal end of an expandable device that is configured to be positioned within intraparenchymal airways can have a lower stiffness than a distal end of the expandable device that is configured to be positioned within extraparenchymal airways.

It may be advantageous for a diameter of an expandable device of the present technology to be at least partially based on a diameter of the airways that the expandable device is configured to be positioned within. Sizing a diameter of an expandable device based on a diameter of the airways can facilitate anchorage and retention of the device, limit damage in the airway wall due to excessive strain, limit granulation tissue formation, and/or improve functional and clinical outcomes. Accordingly, in some embodiments an expandable device of the present technology can have a diameter at least partially based on one or more diameters of the airways in which the device is configured to be positioned. However, as with stiffness, the diameter of the airways varies proximally to distally. While the trachea has a nominal diameter between about 10 mm and about 25 mm in adults, the smallest distal airways have diameters less than 1 mm. Thus, in some embodiments a diameter of the expandable device may vary along a length of the expandable device.

### IV. THERAPEUTIC AGENTS

In any of the above-described embodiments, it may be beneficial to incorporate drug delivery technologies, features and capabilities to counteract an aggressive foreign body response that may, absent such drug delivery, result in occlusion. Broncus Technologies, in development of the Exhale stent for the Airway Bypass procedure, developed a bare metal stent and a paclitaxel-eluting stent. A study in twenty-five dogs demonstrated rapid loss in patency of the bare metal stents and maintenance of patency with the paclitaxel-eluting stents. However, a subsequent human clinical study in over two hundred patients showed that an acute improvement in pulmonary function was not sustained to even thirty days, with stent occlusion suspected as the primary cause of failure. Accordingly, an expandable device possessing a more innovative drug delivery system may be beneficial.

For any of the implants and expandable devices describe herein it may be advantageous to introduce one or more therapeutic agents to address the local healing and/or foreign body responses that may result in full or partial occlusion that undermines the duration of the therapeutic benefit. A utility for controlled, localize drug delivery for a sustained period may preempt or slow the formation of granulation tissue and mucous, thereby mitigating the occlusion risk. This utility may be a formulation of carrier (e.g., polymer, liposome, lipid, etc.) and therapeutic agent that is administered proximate to the treatment site in the airway. This administration of the formulation may occur separately (e.g., needle injection before or after) from the treatment described herein, integrated into the primary procedure (e.g., formulation loaded in the delivery system (e.g., balloon)) or integrated into the implant itself (e.g., expandable device has a polymer-drug coating).

The carrier described herein may adhere to the therapeutic agent to form a matrix. Features may be incorporated into this matrix to achieve a controlled, sustained release of therapeutic agent. One such feature is a releasing agent that is configured to dissolve when contacted by body fluids such that such dissolution will create a porosity of the matrix, thereby allowing for controlled diffusion and release of the therapeutic agent. Suitable releasing agents for use in the present technology include polysorbates, such as Polysorbate 80, Polysorbate 60, Polysorbate 40, and Polysorbate 20; sorbitan fatty acid esters, such as sorbitan monostearate (Span 60), sorbitan tristearate (Span 65), sorbitane trioleate (Span 85), sorbitan monooleate (Span 80), sorbitan monopalmitate, sorbitan monostearate, sorbitan monolaurate, sorbitan monopalmitate, sorbitan trioleate, and sorbitan tribehenate; sucrose esters, such as sucrose monodecanoate, sucrose monolaurate, sucrose distearate, and sucrose stearate; castor oils such as polyethoxylated castor oil, polyoxyl hydrogenated castor oil, polyoxyl 35 castor oil, Polyoxyl 40 Hydrogenated castor oil, Polyoxyl 40 castor oil, Cremophor® RH60, and Cremophor® RH40; polyethylene glycol ester glycerides, such as Labrasol®, Labrifil® 1944; poloxamer; polyoxyethylene polyoxypropylene 1800; polyoxyethylene fatty acid esters, such as Polyoxyl 20 Stearyl Ether, diethylene glycol octadecyl ether, glyceryl monostearate, triglycerol monostearate, Polyoxyl 20 stearate, Polyoxyl 40 stearate, polyoxyethylene sorbitan monoisostearate, polyethylene glycol 40 sorbitan diisostearate; oleic acid; sodium desoxycholate; sodium lauryl sulfate; myristic acid; stearic acid; vitamin E-TPGS (vitamin E d-alpha-tocopherol polyethylene glycol succinate); saturated polyglycolized glycerides, such as Gelucire® 44/14 and and Gelucire® 50/13; and polypropoxylated stearyl alcohols such as Acconon® MC-8 and Acconon® CC-6.

Another such feature is the ratio of therapeutic agent to carrier, which can be 1:10, 1:5, 3:10, 2:5, 1:2, 3:5, 7:10, 4:5, 9:10, 1:1, 10:9, 5:4, 10:7, 5:3, 2:1, 5:2, 10:3, 5:1, 10:1. Another such feature is a substantially impermeable coating of the matrix, wherein this coating shall prevent release of therapeutic agent through only portions of matrix that are uncovered (i.e., directional release). Another such feature is the use of multiple layers of coatings or matrices to control and optimize the release profile of one or more therapeutic agents, wherein each layer has either a substantially impermeable coating, a matrix comprising at least one therapeutic agent and polymer or a matrix without a therapeutic agent.

The therapeutic agent may comprise one or more of the following classes of agents: (a) antiproliferative agents, (b) antimucous agents (c) mucolytic material, (d) corticosteroids, (e) antibiotics, (f) anti-inflammatory agents and (g) antimicrobial agents. Examples of antiproliferative agents include sirolimus (rapamycin), everolimus, zotarolimus, paclitaxel, Taxotere (docetaxel), mitomycin-C, gemcitabine, vincristine (leurocristine) and doxorubicin. Examples of antimucous agents include atropine, ipratropium, tiotropium. Examples of mucolytic material include N-acetylcystine and guifensin. Examples of corticosteroids include cortisone, prednisone, prednisolone, methylprednisolone, dexamethasone, betamethasone, hydrocortisone, and others. Examples of anti-inflammatory agents include steroids, prednisone, betamethasone, cortisone, dexamethasone, hydrocortisone and methylprednisolone, non-steroidal anti-inflammatory drugs (NSAIDs), aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, COX-2 inhibitors, and others.

In some embodiments the therapeutic agent can be an antibiotic, an antifungal, and/or an antimicrobial, wherein the antibiotic, the antifungal, and/or the antimicrobial is selected from at least one of amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, α-defensins, and α-protegrins, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, amphotericin B, and others.

In some embodiments, the expandable device does not include drug-eluting material. This can be useful, for example, to simplify manufacturing and regulatory compliance of the expandable device. Furthermore, as discussed elsewhere in this disclosure, expandable devices in accordance with at least some embodiments of the present technology have one or more other features (e.g., structural and/or performance features) that reduce or eliminate the need for drugs that suppress foreign body response. In these and other cases, the expandable device can include an uncoated wire, such as a bare metal wire.

### V. MODIFYING AIRWAY WALL

In some of the embodiments described herein, it may be advantageous for the expandable device to modify and/or alter the airway wall. In one example, the expandable device comprises self-expanding capabilities (e.g., nitinol construction), whereby deployment of the expandable device results in the application of a chronic outward force to the airway wall that causes a gradual dilation of the airway wall and expansion of the airway lumen. In this example, the self-expansion of the expandable device would cause the airway wall to expand beyond its native diameter. Additionally, or alternatively, expansion of the expandable device can be facilitated by a balloon configured to be inflated to force expansion of the expandable device. Forced expansion of the expandable device via a balloon (incorporated as part of a delivery system or separate from the delivery system) may be advantageous because the size and pressure of the balloon can be adjusted to control the expansion of the expandable device.

Controlled expansion of the expandable device is desirable in that such controlled expansion will allow for controlled modification of the airway wall. In one example, it may be desirable to cause dilation of the airway wall to increase the cross-sectional area of the airway lumen, but without creating substantial injury to the airway wall. An increase in the cross-sectional area would improve expiratory outflow, thereby yielding a therapeutic benefit in emphysema patients. In other examples, it may be desirable to cause greater dilation of the airway wall so as to create tears, perforations and/or fenestrations in the airway wall. These tears, perforations and/or fenestrations may create openings to other pockets of trapped air within the diseased parenchyma adjacent to the airway, thereby improving expiratory outflow and pulmonary function. Moreover, these tears, perforations and/or fenestrations, if substantial enough in size and number, may prevent the occlusion that resulted in previous attempts to release trapped air. As such, the expandable devices disclosed here can have self-expanding and./or balloon expandable features and capabilities to best achieve the desired modification of the airway wall.

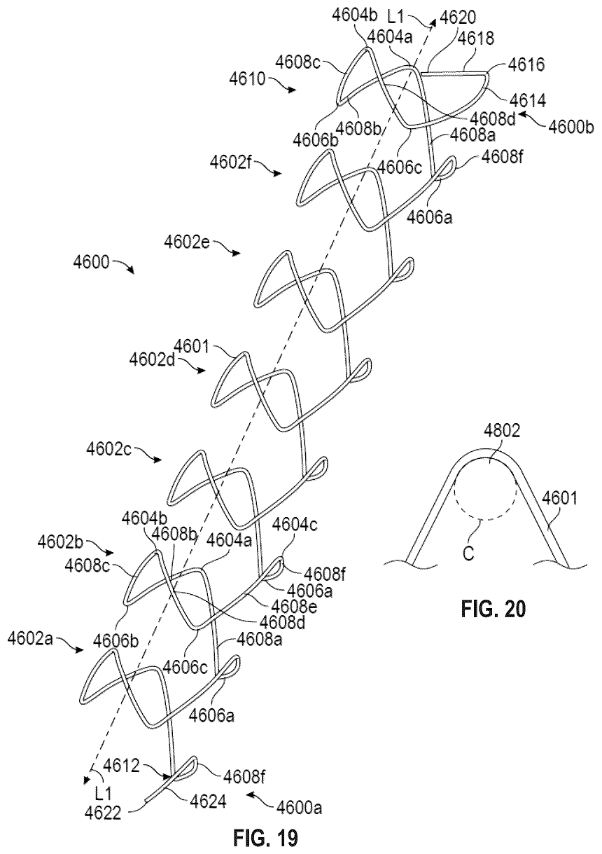

FIG. 19 is a perspective view of an expandable device 4600. In FIG. 19, the device 4600 is shown in an expanded, unconstrained state. The device 4600 has a proximal end portion 4600a, a distal end portion 4600b, and a longitudinal axis L1 extending between the distal and proximal end portions 4600a, 4600b. The device 4600 can comprise a generally tubular structure formed of a wire 4601 wrapped around a longitudinal axis to form a series of bands 4602 (individually labeled as 4602a-4602f), each comprising a 360 degree turn of the wire 4601. The device 4600 further includes a distal structure 4610 distal of the distalmost band 4602f, and a proximal structure 4612 proximal of the proximalmost band 4602a. The wire 4601 undulates between the ends of a given band 4602 such that each band 4602 has a plurality of alternating peaks 4604 (individually labeled as 4604a-4604c) and valleys 4606 (individually labeled as 4606a-4606c) that are connected by struts 4608 (individually labeled as 4608a-4608f). The peaks 4604 can comprise the bend apices within a given band 4602 that are closer to and/or point towards the second end portion 4600b of the device 4600, and the valleys 4606 can comprise the bend apices within a given band 4602 that are closer to and/or point towards the first end portion 4600b of the device 4600. The serpentine configuration of each turn of the wire 4601 makes it easier to radially compress the device 4600 onto and/or into a delivery system, and easier to accurately deploy the device 4600, as discussed in greater detail below.

Along the length of the device 4600, and within a given band 4602, the wire 4601 has struts 4608 that extend both proximally and distally in the direction of the wire turn. For example, following the wire 4601 in a clockwise direction around the turn, the device 4601 has struts 4608 that extend distally, then proximally, then distally, then proximally, then distally, thereby forming a plurality of localized, V-shaped braces that when placed within an airway support the airway wall and serve to tent open the airway lumen. This is in contrast to a simple coil in which the wire extends distally continuously as it wraps around each turn. Such a simple coil may, in some instances, be at greater risk of collapsing or “pancaking” under the radial forces applied by the airway lumen, compared to the device 4600. In some embodiments, for example as shown in FIG. 19, the individual first and fifth struts 4608a and 4608e can be longer than the individual second, third, fourth, and sixth struts 4608b, 4608c, 4608d, and 4608f. In other embodiments the struts 4608 can have different lengths or configurations. Strut length can be measured along the longitudinal axis of the wire 4601. Likewise, the individual second, third, and fourth struts 4608b, 4608c, and 4608d can be longer than the sixth strut 4608f. In some embodiments, the length of the struts 4608 can be determined by the equation 3a−3b=1/pitch, where ‘a’ is the longer strut and ‘b’ is the shorter strut 4608.

As previously mentioned, the bands 4602 are connected to one another only by way of the single, continuous wire. Advantageously, all of the peaks 4604 and valleys 4606 are free peaks and valleys, meaning that none of the peaks 4604 and valleys 4606 are connected to a peak, valley, or other portion of a longitudinally adjacent band 4602. This lack of interconnectedness amongst axially adjacent structures provides the device 4600 with enhanced axial flexibility and stretchability as compared to conventional stents that include one or more bridges or other linkages between longitudinally adjacent struts and/or apices. This flexible configuration enables the device 4600 to stretch and bend with the airway in response to different loads (e.g., bending, torsion, tensile) associated with various anatomical conditions (e.g., airway bifurcation, curvature, etc.) and physiological conditions (e.g., respiration, coughing, etc.), thereby allowing the device to move with the airway to minimize relative motion while still maintaining a threshold radial force. In some embodiments, the device 4600 has a ratio of radial force to longitudinal stiffness that is greater than that of conventional stents. This longitudinal and bending flexibility to move with the airway also has the benefit of limiting relative motion between the device 4600 and the airway wall during respiration and other movements like coughing. Relative motion of the device 4600 to the airway wall can cause inflammation and formation of granulation tissue, which over time can partially or completely occlude the newly-opened lumen, thereby obstructing airflow and frustrating the purpose of treatment. Without being bound by theory, the elimination of longitudinal linkages and/or closed cells along the length of the device 4600 may help maintain perfusion of the treated portion of the airway wall, as closed cells can impede blood flow.

As described herein, there are several aspects of the device that contribute to minimizing granulation tissue formation. One aspect is the self-expanding structure and oversizing relative to the airway diameter that produces a chronic outward force against the airway wall that facilitates wall engagement and apposition, thereby minimizing relative motion. A second aspect is the lack of interconnectedness from the free peaks and valleys that allows for considerable flexibility, thereby allowing the device to move with the airway and minimize relative motion. A third aspect is the low material density and high porosity that cause lesser surface area contact with the airway wall, thereby producing less tissue reaction. A fourth aspect is the wire pattern having no closed cells so as to maintain perfusion, thereby minimizing tissue necrosis and local inflammatory reaction.

Another benefit of the lack of interconnectedness associated with the free peaks and valleys of the expandable device is the low tensile force required to disengage the device from the airway wall. A tensile axial load (i.e., pulling) applied to the wire will cause elongation that reduces the diameter of each loop or band, thereby moving each loop or band away from the airway wall. This separation from the airway wall can facilitate retrievability of the device following implantation with minimal trauma or disturbance to the airway wall.

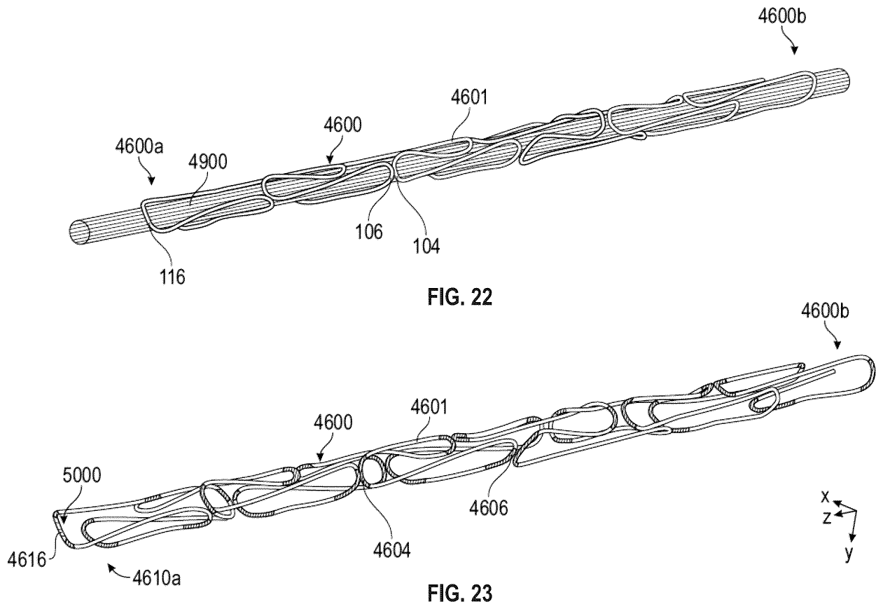

It can be clinically advantageous to place the implant described herein in the distal airway of an emphysematous lung. One historical challenge with conventional, catheter-delivered implants (e.g., stents, braided structures) is the foreshortening that occurs during deployment and implantation. Such foreshortening can make it challenging to accurately deliver the implant to the intended treatment location. Foreshortening is often the result of elongation of the implant during radial compression into a reduced profile for minimally-invasive delivery. Elongation results from the implant's structural design and high material density (i.e., due to the structure and amount of material, the implant cannot stay in the same axial plane when radially compressed). In the device described herein, the lack of longitudinal bridges between axially adjacent structures and relatively low material density (as described below) results in radially compression to a delivery configuration with little to no elongation (e.g., 0%, 5% or less, 10% or less), thereby enabling the device 4600 to be deployed with little to no change in length. Thus, unlike braids and certain stents, the device 4600 does not experience foreshortening when radially expanding. The length of the 4600 device in a compressed, delivery state (for example, see FIG. 22) is substantially the same as the length of the device 4600 in an expanded, unconstrained state. As a result, the device 4600 can be deployed more predictably and with greater landing accuracy.

As shown in FIG. 19, the device 4600 can have a turn density that is measured by the number of full (i.e., 360 degree) turns along an inch of the device 4600. It can be advantageous to have a turn density that is low enough (e.g., adjacent turns are longitudinally farther apart) to allow for sufficient spacing between the adjacent turns and/or bands 4602 of the wire 4601 so that the device 4600 can be compressed onto and/or into a delivery system, and low enough that the resulting surface area contact over the length of the device 4600 does not provoke an adverse tissue response. However, it can also be beneficial to have a turn density that is sufficiently high (e.g., adjacent turns are longitudinally closer together) to prevent sagging and/or invagination of the airway wall between adjacent turns (especially during expiratory flow (e.g., exhalation) when the pressure around the outside of the airway are higher than the pressures within the airway), and to ensure sufficient surface area contact for reducing and/or avoiding relative motion and/or migration. As such, the turn density of the present technology can be optimized for delivery system loadability, minimal invagination of the airway wall between turns, minimal relative motion, and minimal local inflammatory response. In some embodiments, the device 4600 has a turn density of about 1 to about 4 turns per inch. In some embodiments, the device 4600 has a turn density of about 1.2 to about 3.5 turns per inch. In particular embodiments, the device 4600 has a turn density of about 1.8 to about 3 turns per inch. In FIG. 19, the device 4600 has a turn density of 3.

The expanded cross-sectional dimension of the device 4600 may be generally constant or vary along the length of the device 4600 and/or from loop to loop. For example, as discussed herein, the device 4600 can have varying cross-sectional dimensions along its length to accommodate different portions of the airway. For example, in some embodiments the device 4600 can have a diameter that decreases in a distal direction, thereby better approximating the natural distal narrowing of an airway lumen. The diameter may increase in a distal direction gradually over the length of the device 4600, or the device 4600 may have discrete portions with different diameters. For instance, the device 4600 can have a first portion and a second portion along its length. The first portion can have a first cross-sectional dimension that is configured to be positioned in a more distal portion of the airway (such as, for example, in a terminal bronchiole and/or emphysematous areas of destroyed and/or collapsed airways). The second portion can have a second cross-sectional dimension greater than the first cross-sectional dimension and configured to be positioned more proximally (such as in a primary bronchus and/or another portion that has not collapsed). The second portion, for example, can be configured to be positioned in a portion of the airway that is less emphysematous than the collapsed distal portion and/or has cartilage in the airway wall (preferably rings of cartilage and not plates), which can occur at the lobar (generation 2) or segmental (generation 3) level.

In some embodiments, the device 4600 can have a diameter that increases in a distal direction. The diameter may decrease gradually in a proximal direction over the length of the device 4600, or the device 4600 may have discrete portions with different diameters. For instance, the device 4600 can have a generally uniform diameter over much of its length, then a larger diameter over the last distal 1-3 turns (which could be bands 4602 and/or a distal structure 4610). In some embodiments, the device 4600 has a first portion and a second portion along its length. The first portion can have a first cross-sectional dimension that is configured to be positioned in a more distal portion of the airway (such as, for example, in a terminal bronchiole and/or emphysematous areas of destroyed and/or collapsed airways). The second portion can have a second cross-sectional dimension less than the first cross-sectional dimension and configured to be positioned more proximally (such as in a primary bronchus and/or another portion that has not collapsed). The second portion, for example, can be configured to be positioned in a portion of the airway that is less emphysematous than the collapsed distal portion and/or has cartilage in the airway wall (preferably rings of cartilage and not plates), which can occur at the lobar (generation 2) or segmental (generation 3) level. Having an enlarged diameter at a distal portion of the device 4600 can be beneficial for exerting more radial force on the distal airways to produce more dilation, or in some cases even create tears in the airway wall. According to some embodiments, it may be beneficial for the device 4600 to be configured to create tears only along certain portions of the airway engaged by the device 4600. Additionally or alternatively, if the lung is particularly diseased, a distal enlargement might better contact the emphysematous lung and help anchor the device.

In some embodiments, the wire 4601 has a circular cross-sectional shape. In other embodiments, the wire 4601 may have other suitable cross-sectional shapes along its length (e.g., oval, rectangle, square, triangular, polygonal, irregular, etc.). In some embodiments, the cross-sectional shape of the wire 4601 varies along its length. Varying the cross-sectional shape of the wire 4601 may be beneficial to varying the mechanical performance of the device 4600 along its length (e.g., transition from lower to higher radial strength proximal to distal or vice versa). Alternatively or additionally, different cross-sectional shapes allows for different distributions of contact force on the airway wall. For example, a wire having an ovular cross-sectional shape will have greater contact area, wider distribution of contact force and, accordingly, lower contact stress at any point on the device 4600 as compared to a circular cross-section. Without being bound by theory, it is believed that is may be beneficial to utilize a cross-sectional shape with rounded edges, as rounded edges may present a less traumatic surface to the airway wall than straight edges. For example, while a wire having a rectangular cross-sectional shape and linear corners can be used with the present technology, in some cases it may be advantageous to utilize a rectangular wire with curved corners.

The wire 4601 can have a generally constant cross-sectional area along its length, or may have a varying cross-sectional area along its length. It may be beneficial to vary the cross-sectional area of the wire 4601, for example, to vary the radial force and/or flexibility of the device 4600 along its length. For instance, the device 4600 will have a lower radial force and/or be more flexible along portions in which the wire 4601 has a smaller cross-sectional area than along portions in which the wire 4601 has a greater cross-sectional area. In some embodiments, the wire 4601 has a diameter of no more than 0.005 inches, no more than 0.006 inches, no more than 0.007 inches, no more than 0.008 inches, no more than 0.009 inches, no more than 0.01 inches, no more than 0.011 inches, no more than 0.012 inches, no more than 0.013 inches, no more than 0.014 inches, and no more than 0.015 inches.

In some embodiments, the expanded cross-sectional dimension of the device 4600 in an unconstrained, expanded state (i.e., removed from the constraints of a delivery shaft, airway and sitting at rest on a table), can be oversized relative to the diameter of the native airway lumen. For example, the expanded, unconstrained cross-sectional dimension of the device 4600 can be at least 1.5× the original (non-collapsed) diameter of the airway lumen in which it is intended to be positioned. In some embodiments, the device 4600 has an expanded, cross-sectional dimension that is about 1.5× to 6×, 2× to 5×, or 2× to 3× the diameter of the original airway lumen. In some embodiments, it may be clinically beneficial to expand the airway lumen to the greatest diameter possible. A large airway diameter will allow for more efficient release of trapped air, thereby optimizing improvement in pulmonary function (for example, as measured by outflow, FEV, and others). Additionally, there may be clinical benefit in controlled dilation of the airway wall by the implantable device 4600, with or without the aid of an expandable device (e.g., balloon), to create one or more tears in the airway wall to further facilitate the release of air trapped in the surrounding emphysematous lung.

Given that the cartilaginous support in bronchial airways tends to decline proximal to distal, it may be beneficial to have a device with variable turn density, wherein the turn density in the distalmost portion of the device is greater than the turn density in the proximalmost portion of the device. This device configuration, with greater turn density distally and lower turn density proximally, may optionally include lower radial stiffness distally and greater radial stiffness proximally.

The distal structure 4610 is the first portion of the device 4600 to be deployed in the airway lumen. As a result, the distal structure 4610 can be similar to the bands 4602, but adapted to provide greater circumferential force and a soft, atraumatic landing structure. The final apex 4616 of the wire 4601, for example, can be angled so as to orient the distal terminus 4620 of the wire 4601 proximally, and have a greater radius of curvature in its relaxed, unconstrained state than the other apices so as to provide a rounder, softer bend for first contacting the airway wall. In some embodiments, the distal apex 4616 has approximately the same radius of curvature in the relaxed, unconstrained state as the rest of the apices. Additionally or alternatively, the distal terminus 4620 of the wire 4601 can comprise other atraumatic elements, such as a ball (having a cross-sectional dimension only slightly greater than a cross-sectional dimension of the wire 4601) and/or a looped portion of the wire 4601. To enable a greater anchoring force at the distal end portion 4600b of the device 4600, the third valley 4606c of the distal structure 4610 can have a greater radius of curvature so as to substantially align the final apex 4616 (which is a peak) with the second-to-last peak 4604b of the distal structure 4610.

The proximal end portion 4600a of the device 4600 can comprise a single, proximally-extending strut 4624 and a free proximal terminus 4622. Similar to the distal terminus 4620, the proximal terminus 4622 can extend in a proximal direction to limit trauma to the airway wall. The free proximal terminus can also be beneficial for retrieval of the device 4600, if necessary.

The wire 4601 can be any elongated element, such as a wire (e.g., having a circular or ovular cross-sectional shape), a coil, a tube, a filament, a single interwoven elongated element, a plurality of braided and/or twisted elongated elements, a ribbon (have a square or rectangular cross-sectional shape), and/or others. As such, the term “wire,” as used herein, refers to the traditional definition of a wire (e.g., metal drawn out into the form of a thin flexible thread or rod), as well as the other elongated elements detailed herein. The wire 4601 can be cut from a sheet of material then wound around a mandrel into the three-dimensional configuration. In some embodiments, the device 4600 is formed by cutting a tube such that the only remaining portions of the tubular sidewall comprise the wire 4601. The sheet and/or tube can be cut via laser cutting, electrical discharge machining (EDM), chemical etching, water jet, air jet, etc. The wire 4601 can also comprise a thin film formed via a deposition process. The elongated member 102 can be formed using materials such as nitinol, stainless steel, cobalt-chromium alloys (e.g., 35N LT®, MP35N (Fort Wayne Metals, Fort Wayne, Indiana)), Elgiloy, magnesium alloys, tungsten, tantalum, platinum, rhodium, palladium, gold, silver, or combinations thereof, or one or more polymers, or combinations of polymers and metals. In some embodiments, the wire 4601 may include one or more drawn-filled tube (“DFT”) wires comprising an inner material surrounded by a different outer material. The inner material, for example, may be radiopaque material, and the outer material may be a superelastic material.

The cross-sectional area of the wire 4601 can be selected based on several factors, such as turn density, radial force, and ability to radially compress for delivery. All else equal (such as turn density, length of wire, wire material, etc.), the greater the cross-sectional area of the wire 4601, the greater the radial force exerted on the airway wall. However, the greater the cross-sectional area of the wire 4601 and associated radial force, the more difficult it is to compress the device 4600 into and/or onto a delivery system. As such, the wire 4601 of the present technology has a cross-sectional area that, along with the turn density of the wire 4601, provides the device 4600 with a radial force sufficient to maintain airway patency, resist strain and associated cycle fatigue from anatomical loading during respiration and coughing and reduce and/or eliminate relative motion while still allowing the device 4600 to be compressed down to a diameter of less than 3 mm, and in some cases less than 2 mm.

It can be advantageous to have a radial force high enough to resist migration and, via improved wall apposition, reduce relative motion between the device 4600 and the airway wall, as relative motion can irritate the wall tissue and cause a foreign body response that may contribute to occlusion of the airway. The radial force must also be sufficient to maintain patency of the airway, and in some cases dilate the airway to a diameter that is larger than the native diameter of the airway, for example this could be 2-3 times greater. The radial force exerted by the device 4600 on the airway wall is determined, at least in part, by the turn density of the device 4600 and the cross-sectional area of the wire 4601. For example, the greater the cross-sectional area of the wire 4601, the greater the radial force. The greater the turn density of the device 4600, the greater the radial force. Likewise, the lower the cross-sectional area of the wire 4601, the lower the radial force. The lower the turn density of the device 4600, the lower the radial force. The devices 4600 of the present technology can have a radial force per unit length of no more than 7 g/mm, no more than 6 g/mm, no more than 5 g/mm, no more than 4 g/mm, no more than 3 g/mm, no more than 2 g/mm, or no more than 1 g/mm. In some embodiments, the device 4600 has a radial force per unit length of from about 1 to about 5 g/mm. The radial force required to hold open a collapsed airway and maintain patency during respiration is less than that required by stents used to push or hold back tumor growth or atherosclerosis. Such conventional stents typically have a radial force per unit length of about 10 g/mm or greater.

The device 4600 may be configured to have minimal surface area contact with the airway wall to reduce the amount of foreign body response (such as inflammation and granulation tissue) and risk of airway occlusion. As used in this discussion, “contacting surface area” refers to the surface area of the portion of the device 4600 that contacts the inner surface of the airway wall, which is less than the total surface area of the wire 4601. Minimizing the contacting surface area of the device 4600 can also be beneficial for limiting and/or avoiding occlusion of other distal branch openings, and for enabling more efficient mucociliary clearance. The contacting surface area of the device 4600, however, also impacts the device's ability to resist migration and relative motion. As such, the devices 4600 of the present technology can be configured to have a contacting surface area that is low enough to minimize (or localize) an adverse tissue reaction and allow for sufficient mucociliary clearance, but high enough to provide good contact with the airway and resist motion. The devices 4600 of the present technology can have, for example, a contacting surface area of no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, or no more than 5%. Said another way, the porosity of the device 4600 can be at least 80%, at least 81%, at least 82%, at least 83%, at least 84%, least 85%, at least 86%, at least 87%, at least 88%, at least 89%, at least 90%, at least 91%, at least 92%, at least 93%, at least 94%, or at least 95%.

In some embodiments, regardless of whether the wire 4601 is made of and/or includes a radiopaque material, the device 4600 can include one or more radiopaque markers. The radiopaque markers, for example, can be disposed at one or both ends of the device 4600 to facilitate accurate positioning and placement.

It can be advantageous to configure the device 4600 such that when implanted in an airway lumen to expand the lumen cross-sectional area, the resulting airway lumen is biomimetic as possible to a healthy airway lumen. For example, the device 4600 can be configured so as to sufficiently dilate the airway lumen, yet not impose an overly artificial and/or unnatural shape on the airway lumen (e.g., straight or cylindrical airway lumen), such that the treated airway lumen is advantageously maintaining to some extent the inherent curving or tortuosity of the axis of the airway lumen. Thus, over an extended period of time (e.g., over three months, over six months, over a year, over eighteen months, over twenty four months, etc.) the treated airway lumen can remodel itself into a more natural, curved shape, thereby resulting in improved clinical outcomes.

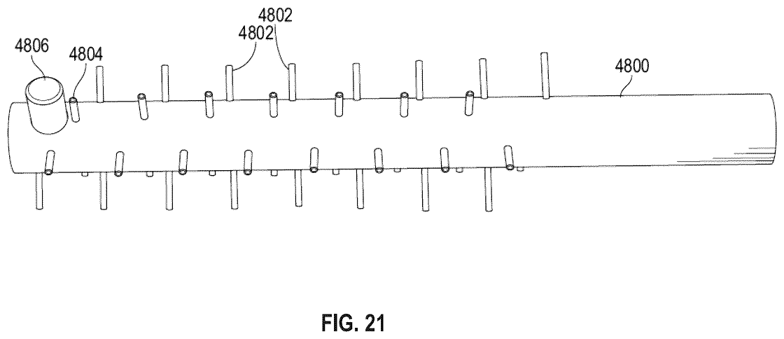

In some embodiments, the device 4600 is manufactured by wrapping the wire 4601 around a mandrel according to a predetermined wrap pattern, then heat setting the wire 4601 while held in place on the mandrel so that when the wire 4601 is removed from the mandrel, the wire 4601 substantially maintains its on-mandrel shape. FIG. 21 shows a mandrel 4800 configured for use in manufacturing the devices of the present technology. As shown in FIG. 21, the mandrel 4800 can be generally cylindrical and include a plurality of posts 4802 extending radially away from an outer surface of the mandrel 4800. The posts 4802 can be arranged in a pattern that produces a desired wrap geometry. The radius of curvature of the posts 4802, for example, can determine the radius of curvature of the apices. FIG. 20 shows a portion of the wire 4601 wrapped around one of the posts 302. Different apices along the device 4600 can have the same radii of curvature or different radii of curvature.

In some cases it may be beneficial to use posts having a radius of curvature that closely resembles a shape of the apices when the device 4600 is compressed down onto and/or into a delivery system. FIG. 22 shows the device 4600 in a radially compressed state, positioned over an elongated delivery member 4900. As the device 4600 gets radially compressed, the two struts 4608 adjacent any given peak 4604 or valley 4606 get pinched together, thereby placing a strain on the attached apex. FIG. 23, for example, shows a finite element analysis performed on the device 4600 to calculate cyclic strains, since the device 4600, when implanted, will exhibit cyclic strain in the form of respiration, coughing, and others. As shown in FIG. 23, the strain amplitude peaked at the distal portion where the apex 4616 was heat set to have a radius of curvature that was greater than that of the other apices (such as peak 4604 and valley 4606). The apices that were heat set around smaller diameter posts (having small radii of curvature) were projected to experience less strain and fatigue compared to the distal apex 4616 when forced into a compressed state. Accordingly, it may be desirable for the apices to have an average radius of curvature that is no greater than 2.5 mm (e.g., 2.5 mm or less, 2 mm or less, 1 mm or less, 0.5 mm or less, or within a range from 0.35 mm to 0.60 mm).

Additional examples of expandable devices, systems, and methods for treating COPD and/or devices, systems, and methods for modifying an airway wall can be found, for example, in U.S. Pat. No. 9,592,138, filed Sep. 13, 2015, titled PULMONARY AIRFLOW, and PCT International Application No. PCT/US2022/073962, filed Jul. 20, 2022, titled ENDOBROCHIAL IMPLANTS AND RELATED TECHNOLOGY, each of which is incorporated by reference herein in its entirety.

### VI. ADDITIONAL EXAMPLES

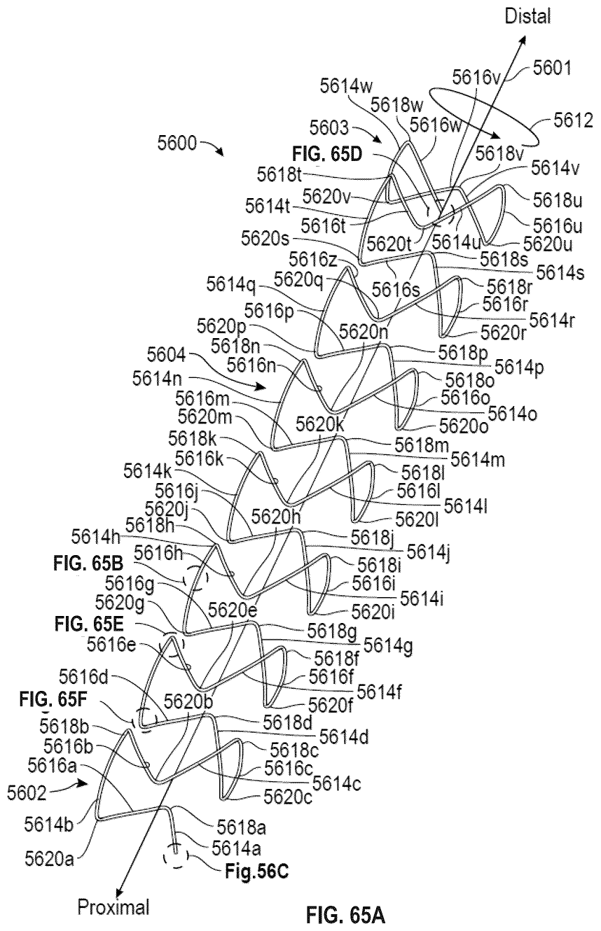

FIGS. 65A, 66A and 67 are a perspective view, an end view, and a profile view, respectively of the implant 5600 in accordance with at least some embodiments of the present technology. FIGS. 65B-65F are callouts corresponding to FIG. 65A. FIG. 66B is a callout corresponding to FIG. 66A. In FIGS. 65A-67, the implant 5600 is in an unconstrained state. This can be a state in which the implant 5600 assumes in the absence of external sources of constraint, such as a sheath during delivery of the implant 5600 or a wall of a bronchial tree after deployment of the implant 5600. Features of the implant 5600 are described herein with respect to the implant 5600 in this unconstrained state unless otherwise specified. The implant 5600 can be elongate with a longitudinal axis 5601. The implant 5600 can include a proximal end portion 5602 and a distal end portion 5603 spaced apart from one another along the longitudinal axis 5601. Between the proximal end portion 5602 and the distal end portion 5603 along the longitudinal axis 5601, the implant 5600 can include an intermediate portion 5604. The overall implant 5600 can be configured to deployed at a treatment location within a bronchial tree of a human subject. Aspects of examples of this deployment are described in detail below. In at least some cases, the proximal end portion 5602 and the distal end portion 5603 are configured to be deployed at different respective airways. For example, the proximal end portion 5602 can be configured to be deployed at a first airway and the distal end portion 5603 can be configured to be deployed at a second airway of a generation greater than a generation of the first airway. The respective generations of the first and second airways can be different by 1, 2, 3, 4, 5, 6, or an even greater number depending on features such as the length and diameter of the implant 5600. The first airway can be of a generation 2 or greater, such as 2, 3, 4, 5 or 6.

The implant 5600 can further include a wire 5605 extending along a wire path 5606. The wire path 5606 can extend between a first end 5607 at the proximal end portion 5602 and an opposite second end 5608 at the distal end portion 5603. The wire path 5606 can be continuous between the first end 5607 and the second end 5608. Furthermore, the wire 5605 can include a first terminus 5609 at the first end 5607 and a second terminus 5610 at the second end 5608. The wire path 5606 can extend in a circumferential direction 5612 about the longitudinal axis 5601. Some, most, or all of the wire 5605 and the wire path 5606 can be within a tubular region 5611 coaxially aligned with the longitudinal axis 5601. In the illustrated embodiment, the tubular region 5611 has a circular cross-sectional shape perpendicular to the longitudinal axis 5601. In other embodiments, a counterpart of the tubular region 5611 can be ovoid, triangular with rounded corners, square with rounded corners, otherwise polygonal with rounded corners, or have another suitable shape perpendicular to a counterpart of the longitudinal axis 5601. Furthermore, although the longitudinal axis 5601 and the tubular region 5611 are straight in the illustrated embodiment, in other embodiments, the longitudinal axis 5601 and the tubular region 5611 can be curved. For example, a counterpart of the implant 5600 can be curved, angled, serpentine, or have another suitable nonlinear shape. Such a nonlinear shape, for example, can be selected to correspond to a shape of an airway region in which the counterpart of the implant 5600 is to be deployed.

The overall wire path 5606 between the first end 5607 and the second end 5608 include includes seven complete turns about the longitudinal axis 5601. In other embodiments, a counterpart of the wire path 5606 can include another suitable number of turns, such as another suitable number of turns corresponding to a desired pitch and overall length of a counterpart of the implant 5600. In at least some embodiments, the wire path 5606 at the intermediate portion 5604 includes three or more complete turns, such as four turns, five turns, six turns, or more. In these and other embodiments, the wire path 5606 at the proximal end portion 5602 can include one complete turn closest to the first end 5607. Similarly, the wire path 5606 at the distal end portion 5603 can include one complete turn closest to the second end 5608. Delineation between the proximal end portion 5602, the distal end portion 5603, and the intermediate portion 5604 can be based on turns and/or based on segments of the longitudinal axis 5601. For example, the proximal end portion 5602 can be coextensive with a proximalmost 10% of the longitudinal axis 5601, the distal end portion 5603 can be coextensive with a distalmost 10% of the longitudinal axis 5601, and the intermediate portion can be coextensive with an intermediate 80% longitudinal axis 5601. Alternatively, the proximal end portion 5602 can be coextensive with a proximalmost 15% of the longitudinal axis 5601, the distal end portion 5603 can be coextensive with a distalmost 15% of the longitudinal axis 5601, and the intermediate portion can be coextensive with an intermediate 70% longitudinal axis 5601. Other suitable delineations are also possible.

The wire 5605 can include first legs 5614 (individually identified as first legs 5614a-5614w) and second legs 5616 (individually identified as second legs 5616a-5616w) alternatingly disposed along the wire path 5606. The first legs 5614a-5614w can extend distally in the circumferential direction 5612 while the second legs 5616a-5616w extend proximally in the circumferential direction 5612. In the illustrated embodiment, all of the first legs 5614a-5614w and all of the second legs 5616a-5616w have these specified orientations. In other embodiments, a counterpart of the wire 5605 can include only some (e.g., most, all but one, all but two, etc.) counterparts of the first legs 5614a-5614w and/or counterparts of the second legs 5616a-5616w having the specified orientations. For example a counterpart of the wire 5605 can include counterparts of the first legs 5614a-5614w and counterparts of the second legs 5616a-5616w having the specified orientations only at a counterpart of the intermediate portion 5604, but not at a counterpart of the proximal end portion 5602 and/or not at a counterpart of the distal end portion 5603. Furthermore, in the illustrated embodiment and in at least some other embodiments, the first legs 5614a-5614w and the second legs 5616a-5616w and counterparts thereof can have any suitable features of corresponding portions of other devices described herein.

The wire 5605 can include first apex portions 5618 (individually identified as first apex portions 5618a-5618w) disposed at respective first apex points 5619 along the wire path 5606. The wire 5605 can also include second apex portions 5620 (individually identified as second apex portions 5620a-5620v) disposed at respective second apex points 5621 along the wire path 5606. In at least some cases, the first legs 5614a-5614w and the second legs 5616a-5616w are alternatingly disposed along the wire path 5606. Furthermore, the first legs 5614a-5614w and the second legs 5616a-5616w can be interspersed among the first apex portions 5618a-5618w and the second apex portions 5620a-5620v along the wire path 5606. As shown in FIG. 65A, the first apex portions 5618a-5618w can point distally (i.e., more toward the distal end portion 5603 than toward the proximal end portion 5602 along the longitudinal axis 5601). Correspondingly, portions of the wire 5605 nearest to the first apex portions 5618a-5618w can extend away from the first apex portions 5618a-5618w proximally. Similarly, the second apex portions 5620a-5620v can point proximally (i.e., more toward the proximal end portion 5602 than toward the distal end portion 5603 along the longitudinal axis 5601). Correspondingly, portions of the wire 5605 nearest to the second apex portions 5620a-5620v can extend away from the second apex portions 5620a-5620v distally. In the illustrated embodiment and in at least some other embodiments, the first apex portions 5618a-5618w and the second apex portions 5620a-5620v and counterparts thereof can have any suitable features of corresponding portions of other devices described herein.

The overall implant 5600, the proximal end portion 5602, the distal end portion 5603, and/or the intermediate portion 5604 can consist essentially of the wire 5605. Furthermore, the wire 5605 throughout the implant 5600, at the proximal end portion 5602, at the distal end portion 5603, and/or at the intermediate portion 5604 can consist essentially of various combinations of the first legs 5614a-5614w, the second legs 5616a-5616w, the first apex portions 5618a-5618w, and the second apex portions 5620a-5620v. In the illustrated embodiment, the proximal end portion 5602 includes the four of the first legs 5614 (first legs 5614a-5614d), three of the second legs 5616 (second legs 5616a-5616c), three of the first apex portions 5618 (the first apex portions 5618a-5618c), and three of the second apex portions 5620 (the second apex portions 5620a-5620c). These components correspond to a portion of the wire 5605 extending along a single complete turn of the wire path 5606 closest to the first end 5607 but with the first leg 5614d extending slightly beyond this turn along the wire path 5606 toward the second end 5608. In the illustrated embodiment, the distal end portion 5603 includes three of the first legs 5614 (first legs 5614u-5614w), three of the second legs 5616 (second legs 5616u-5616w), three of the first apex portions 5618 (the first apex portions 5618u-5618w), and two of the second apex portions 5620 (the second apex portions 5620u-5620v). These components correspond to a portion of the wire 5605 extending along a single complete turn of the wire path 5606 closest to the second end 5608 but with the second leg 5616u extending slightly beyond this turn along the wire path 5606 toward the first end 5607. Finally, in the illustrated embodiment, the intermediate portion 5604 includes 16 of the first legs 5614 (the first legs 5614e-5614t), 17 of the second legs 5616 (the second legs 5616d-5616t), 17 of the first apex portions 5618 (the first apex portions 5618d-5618t), and 17 of the second apex portions (the second apex portions 5620d-5620t). These components correspond to a portion of the wire 5605 extending along five complete turns of the wire path 5606. In other embodiments, as discussed above, counterparts of the proximal end portion 5602, the distal end portion 5603, and the intermediate portion 5604 can have other suitable delineations. Furthermore, these counterparts can include other suitable quantities and/or types of components.

In at least some cases, the wire 5605 is unbranched throughout the wire path 5606. For example, the wire 5605 can lack bifurcations, trifurcations, or other types of junctions at which the wire 5605 divides. In addition or alternatively, the wire 5605 can be untethered throughout the wire path 5606. For example, the wire 5605 can lack bridges or other structural connections between different portions of the wire 5605 spaced apart from one another along the wire path 5606 and/or between the wire 5605 and other implant components. By way of nonbinding theory, these features alone or in combination with other features described herein may be useful to reduce a foreign body response associated with the implant 5600, to increase longitudinal flexibility of the implant 5600, and/or for one or more other reasons. In other embodiments, a counterpart of the wire 5605 can be branched, tethered, and/or present with other implant components.

With reference again to FIGS. 65A-67, the first terminus 5609 and/or the second terminus 5610 can be untethered. In contrast, wire ends in conventional implants are typically tethered in some manner, such as by being tied or otherwise bonded to other wire portions. This tethering is intuitive because untethered wire ends are conventionally assumed to have greater potential than tethered wire ends to cause trauma, to migrate, and/or to exhibit other undesirable behaviors after implant deployment. The inventors recognized that making the first terminus 5609 and/or the second terminus 5610 untethered had potential benefits and that associated problems could be mitigated or even eliminated with other implant features. Among the benefits is supporting mucociliary clearance. The inventors recognized that a lack of branching and/or tethering at other portions of the wire 5605 and/or the lack of structures of the implant 5600 other than the wire 5605, as discussed above, can also support this objective. Moreover, without wishing to be bound to this theory, the inventors identified mucociliary clearance as useful for supporting long-term use of the implant 5600 without loss of airway patency due to mucus impaction or the accumulation of granulation tissue. Accordingly, the implant 5600 can be configured to allow mucociliary clearance from a location immediately distal to the implant 5600 to a location immediately proximal to the implant 5600 while the implant 5600 is deployed at a treatment location within a bronchial tree.

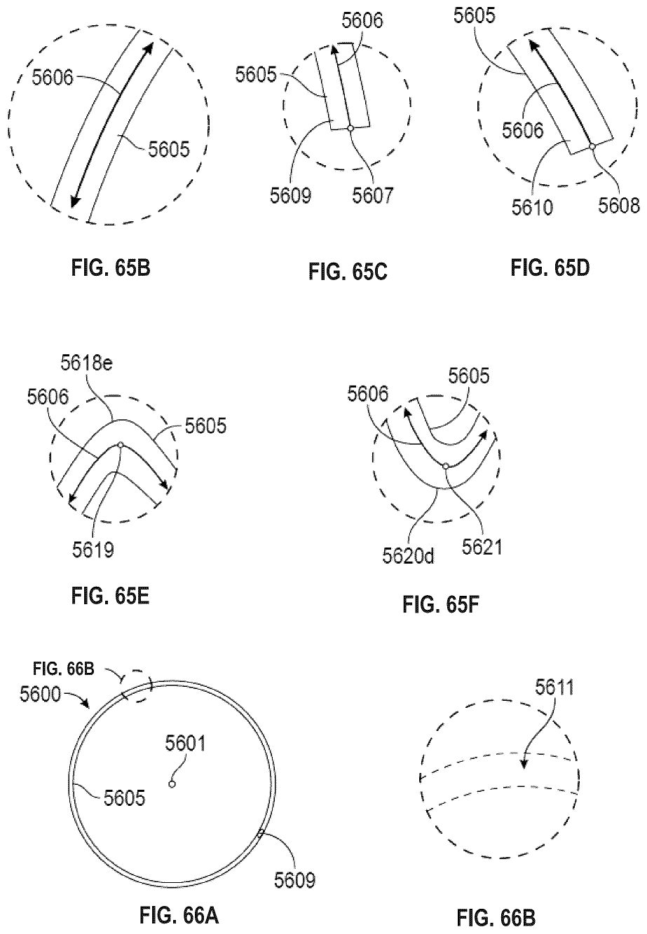

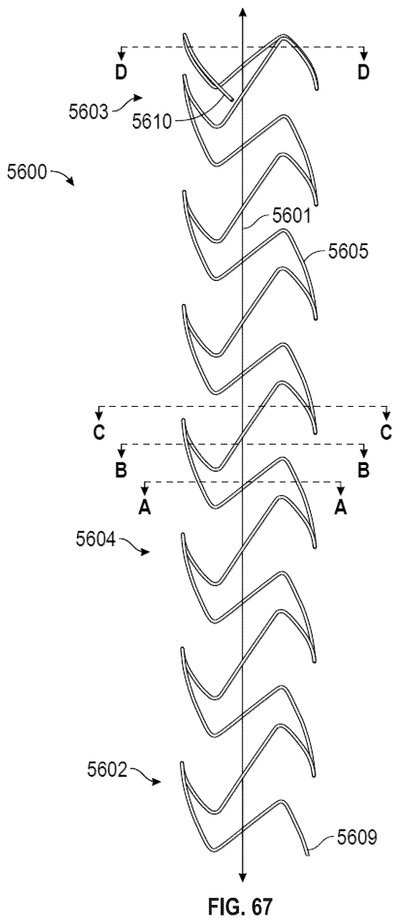

As best shown in FIG. 67, the first terminus 5609 can be at a proximalmost end of the implant 5600. Correspondingly, the implant 5600 can include a given one of the first legs 5614 at the first end 5607 of the wire path 5606. Furthermore, a pitch of the wire path 5606 at the proximal end portion 5602 can be about the same as (e.g., within 10% of) a pitch of the wire path 5606 at the intermediate portion 5604. These features and a lack of tethering at the first terminus alone or in combination can facilitate retrievability of the implant 5600. For example, although the implant 5600 is expected to be suitable for indefinite use, in some cases it may be useful to remove the implant 5600 from a treatment location after deployment. This may be the case, for example, when a clinician deploys the implant 5600 improperly or when unexpected and unusual biological processes cause an airway region in which the implant 5600 is deployed to eventually lose patency. Retrieving the implant 5600 can include gripping the wire 5605 at or near the first terminus 5609 and pulling the wire 5605 proximally. The described features of the first terminus 5609 can facilitate gripping access and can help guide the wire 5605 away from airway walls in response to pulling force. For example, the implant 5600 generally and the proximal end portion 5602 particularly can be configured to unwind and elongate rather than maintain the same shape perpendicular to the longitudinal axis 5601 during retrieval. Accordingly, rather than dragging across the airway walls proximally, the implant 5600 can tend to disengage inwardly and then move proximally during retrieval. This can reduce or eliminate excess trauma.

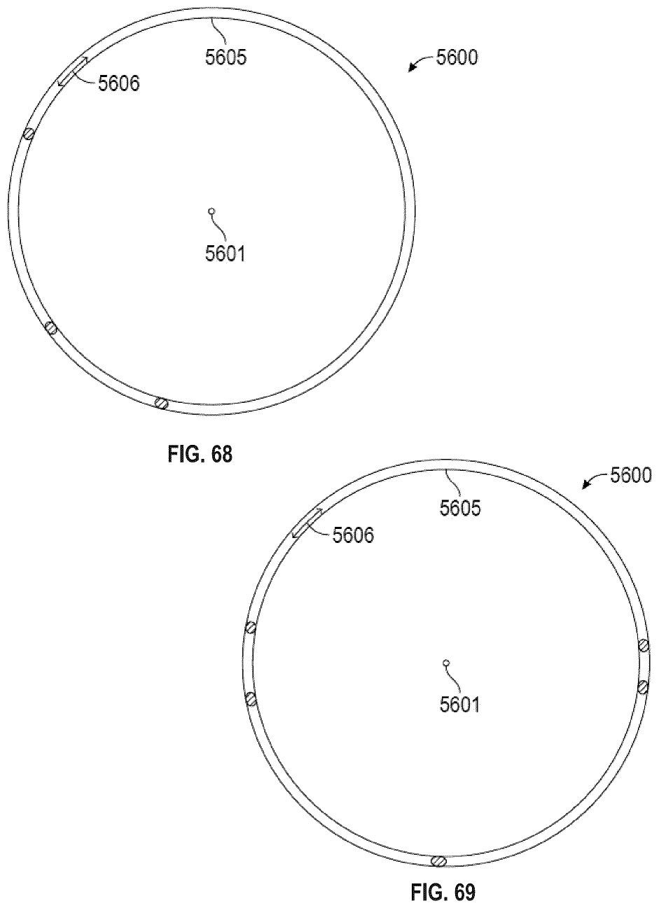

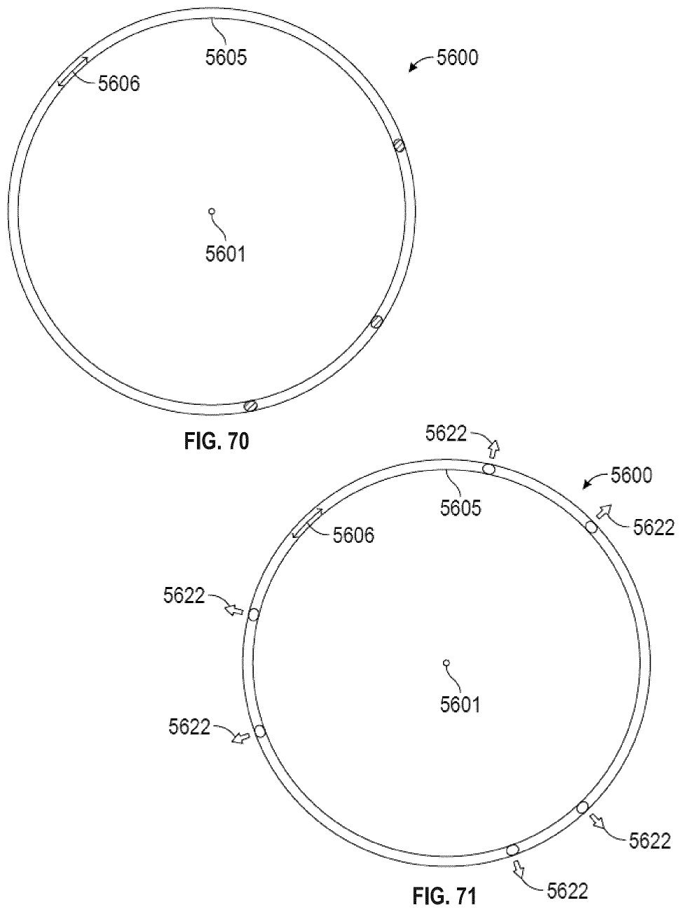

FIGS. 68, 69, 70 and 71 are cross-sectional views of the implant 5600 taken along lines A-A, B-B, C-C, and D-D in FIG. 67, respectively. As shown in FIGS. 68-71, planes perpendicular to the longitudinal axis 5601 at different portions of the implant 5600 can intersect more than one circumferentially spaced apart portion of the implant 5600. This contrasts with a simple coil. The inventors have discovered that contacting more than one circumferentially spaced apart portions of a wall of an airway region can be useful for establishing and maintaining airway patency. Portions of the implant 5600 that a plane perpendicular to the longitudinal axis 5601 intersects can correspond to portions of the implant 5600 that contact a wall of an airway region when the implant 5600 is deployed. Accordingly, the implant 5600 can contact three circumferentially spaced apart portions of a wall of an airway region at a plane perpendicular to the longitudinal axis 5601 at the line A-A, five such portions at the line B-B, three such portions at the line C-C, and six such portions at the line D-D. Lines A-A, B-B, and C-C are at the intermediate portion 5604 whereas line D-D is at the distal end portion 5603. In at least some cases, any given plane perpendicular to the longitudinal axis 5601 at the intermediate portion 5604 and/or a middle 50% of a length of the implant 5600 along the longitudinal axis 5601 intersects at least three (e.g., from three to five) circumferentially spaced apart points along the wire path 5606.

As FIGS. 68-71 suggest, the implant 5600 can be configured to contact more circumferentially spaced apart portions of a wall of an airway region at planes perpendicular to the longitudinal axis 5601 at the distal end portion 5603 than at planes perpendicular to the longitudinal axis 5601 at the intermediate portion 5604. For example, the implant 5600 can be configured to intersect at least a first number of circumferentially spaced apart points along the wire path 5606 at any given plane perpendicular to a middle 50% of a length of the implant 5600 along the longitudinal axis 5601 and to intersect at least a greater second number of circumferentially spaced apart points along the wire path 5606 at any given plane perpendicular to distalmost 5% of the length of the implant 5600 along the longitudinal axis 5601. In at least some cases, the second number of circumferentially spaced apart points is at least five. Furthermore, among the circumferentially spaced apart points along the wire path 5606 at which any given plane perpendicular to distalmost 5% of the length of the implant 5600 along the longitudinal axis 5601 intersects the implant, a maximum circumferential spacing between any circumferentially neighboring pair of the points can be no more than 180 degrees, such as no more than 120 degrees. Conversely, for at least one neighboring pair of circumferentially spaced apart points, there may be a minimum circumferential spacing of at least 60 degrees, such as at least 90 degrees, 120 degrees, or 150 degrees.

The inventors recognized a relatively large number of and/or relatively circumferentially balanced positioning of points of contact between the distal end portion 5603 and an airway region as potentially useful to facilitate deployment of the implant 5600. For example, in at least some cases, the implant 5600 is deployed by causing relative movement between a sheath and the implant 5600 such that the implant 5600 is gradually uncovered and allowed to expand radially. In these and other cases, the distal end portion 5603 can expand before other portions of the implant 5600. When this expansion begins, the distal end portion 5603 may have no established connection to the airway region. If a counterpart of the distal end portion 5603 initiated and/or propagated connection with an airway region at a single point, the force exerted against the airway region at that point would potentially cause asymmetrical expansion of the airway region. This, in turn, would potentially cause the counterpart of the distal end portion 5603 to move unpredictably during deployment, leading to potential trauma and/or suboptimal control over positioning. In contrast, with reference again to FIG. 71, the distal end portion 5603 can be configured to exert force (corresponding to arrows 5622) at a sufficient number of circumferentially spaced apart portions of the airway region to cause the airway region to expand relatively uniformly, thereby reducing potential trauma and/or enhancing control over positioning. After its deployment, the distal end portion 5603 can anchor the implant 5600 such that further radial expansion of the implant 5600 does not cause trauma or unduly compromise control over positioning of the implant 5600 even if such further expansion propagates along a relatively small number of points and/or points that are relatively circumferentially unbalanced.

### VII. IMPLANT GEOMETRY AND CONTACT DENSITY

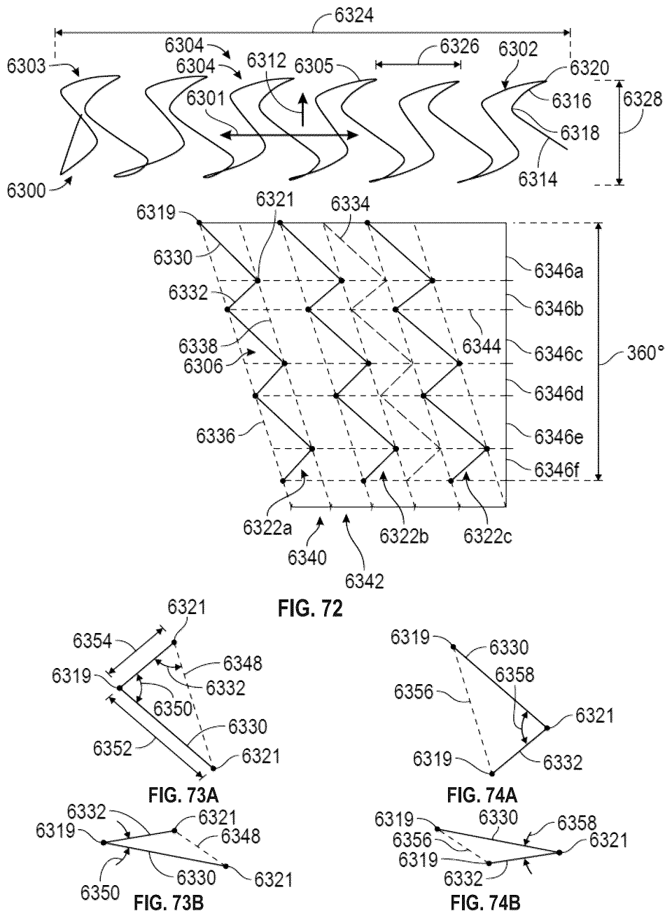

FIG. 72 is a profile view of an implant 6300 in accordance with at least some embodiments of the present technology in an unconstrained state juxtaposed with a schematic diagram illustrating certain geometrical aspects of the implant 6300. The implant 6300 is generally similar to the implant 5600 described above except that the implant 6300 has fewer turns and different wire termination features. The implant 6300 can include or define a longitudinal axis 6301, a proximal end portion 6302, a distal end portion 6303, a intermediate portion 6304, a wire 6305, a wire path 6306, a circumferential direction 6312 (as indicated and curving into the page), first legs 6314, second legs 6316, first apex portions 6318, first apex points 6319, second apex portions 6320, and second apex points 6321 at least generally corresponding to the longitudinal axis 5601, the proximal end portion 5602, the distal end portion 5603, the intermediate portion 5604, the wire 5605, the wire path 5606, the circumferential direction 5612, the first legs 5614, the second legs 5616, the first apex portions 5618, the first apex points 5619, the second apex portions 5620, and the second apex points 5621, respectively, of the implant 5600.

With reference now to FIG. 72, the wire path 6306 is shown in a two-dimensional unwound representation with portions of the wire path 6306 corresponding to three successive turns 6322 (individually identified at turns 6322a-6322c) of the wire path 6306 at the intermediate portion 6304. The vertical axis in the schematic diagram corresponds to circumferential position and spacing in the circumferential direction 6312 about the longitudinal axis 6301. The horizontal axis in the schematic diagram corresponds to longitudinal position and spacing along the longitudinal axis 6301. The implant 6300 can define a length 6324 along the longitudinal axis 6301, a pitch 6326 along the longitudinal axis 6301, and a diameter 6328 perpendicular to the longitudinal axis 6301. In the schematic diagram, first segments 6330 of the wire path 6306 correspond to lengths of the first legs 6314. Similarly, second segments 6332 of the wire path 6306 correspond to lengths of the second legs 6316. For the sake of simplicity, the first and second segments 6330, 6332 are represented as straight lines between neighboring first and second apex points.

In the illustrated embodiment, the length 6324 is about 50 mm, the average pitch 6326 at the intermediate portion 6304 is about 8.1 mm, and the average diameter 6328 is about 10 mm. In other embodiments, these dimensions can be different. For example, a counterpart of the length 6324 can be within a range from 50 mm to 200 mm, such as from 70 mm to 200 mm or from 70 mm to 120 mm. Alternatively, a counterpart of the length 6324 can be less than 50 mm or greater than 200 mm. A counterpart of the average pitch 6326 at the intermediate portion 6304 can be within a range from 4 mm to 12 mm, such as from 6 mm to 12 mm, or from 6 mm to 10 mm. Alternatively, a counterpart of the average pitch 6326 can be less than 4 mm or greater than 12 mm. A counterpart of the average diameter 6328 can be within a range from 2 mm to 20 mm, such as from 4 mm to 20 mm, or from 5 mm to 15 mm. Alternatively, a counterpart of the average diameter 6328 can be less than 2 mm or greater than 20 mm. In other embodiments, counterparts of the implant 6300 can have still other suitable dimensions.

With reference again to the illustrated embodiment, the average pitch 6326 at the distal end portion 6303 can be smaller than the average pitch 6326 at the intermediate portion 6304 and smaller (e.g., from 10% to 50% smaller) than the average pitch 6326 at the proximal end portion 6302. This pitch difference can correspond to a greater number of circumferentially spaced apart portions of the wire 6305 along which contact between the implant 6300 and an airway wall simultaneously propagates during deployment of the distal end portion 6303 relative to deployment of the intermediate portion 6304. In addition or alternatively, this pitch difference can correspond to a greater degree of circumferential balance among portions of the wire 6305 along which contact between the implant 6300 and an airway wall simultaneously propagates during deployment of the distal end portion 6303 relative to deployment of the intermediate portion 6304. As discussed above, the number of contact portions and/or the circumferential balance of these contact portions can be useful to reduce potential trauma and/or enhance control over positioning during implant deployment.

The pitch 6326 can also be relevant to performance characteristics of the implant 6300, such as enhancing mucociliary clearance. In at least some cases, the implant 6300 is configured to define an unobstructed mucociliary clearance region extending along a continuous mucociliary clearance path 6334 from the location immediately distal to the implant 6300 to the location immediately proximal to the implant 6300 while the implant 6300 is deployed at a treatment location within a bronchial tree of a human subject. As shown in FIG. 72, the mucociliary clearance path 6334 can extend between successive turns of the wire path 6306. An average width of the mucociliary clearance region parallel to the longitudinal axis 6301 can be significantly greater than an average cross-sectional diameter of the wire 6305 perpendicular to the wire path 6306. This can correspond to a synergistic combination of relatively small contact area between the implant 6300 and an airway wall thereby a foreign body response and relatively large area available for mucociliary clearance. These features alone or together can increase the time (potentially indefinitely) during which an airway region in which the implant 6300 is deployed remains patent. In at least some cases, the average width of the mucociliary clearance region parallel to the longitudinal axis 6301 is at least 10 times (e.g., within a range from 10 times to 20 times) the average cross-sectional diameter of the wire 6305 perpendicular to the wire path 6306. In addition or alternatively, the average pitch 6326 can be within a range from 50% to 110% (e.g., from 70% to 90%) of the average diameter 6328. This can be the case, for example, at the intermediate portion 6304 and/or throughout the implant 6300.

The implant 6300 can be configured to resiliently transition from a low-profile delivery state to an expanded deployed state. The average diameter 6328 can be significantly different between these states. By way of nonbinding theory, the inventors have found that this feature has great potential to facilitate establishing and maintaining airway patency. Expansion of an airway well beyond its native diameter creates a relatively large free-passage area that is less likely or at least slower to become occluded due to mucus impaction or the accumulation of granulation tissue. In some embodiments, the average diameter 6328 when the implant 6300 is in the deployed state is at least 3 times (e.g., at least 3.5 times, at least 4 times, at least 4.5 times, or at least 5 times) the average diameter 6328 when the implant 6300 is in the delivery state. In these and other embodiments, the average diameter 6328 when the implant 6300 is in the illustrated unconstrained state is at least 4 times (e.g., at least 4.5 times, at least 5 times, at least 5.5 times, or at least 6 times) the average diameter 6328 when the implant 6300 is in the delivery state. Furthermore, a ratio of the average diameter 6328 to the length 6324 can be within a range from 1:5 to 1:30, such as from 1:10 to 1:30.

In the illustrated embodiment, the diameter 6328 is consistent throughout the length 6324. In at least some cases, the diameter 6328 varies no more than 5% or no more than 10% throughout the length 6324. Relatedly an average of the diameter 6328 at the proximal end portion 6302 can be no more than 5% different or no more than 10% different than an average of the diameter 6328 at the distal end portion 6303. This may be counterintuitive because the distal end portion 6303 is configured to be deployed at a more distal portion of a bronchial tree than the portion at which the proximal end portion 6302 is deployed. More distal airway regions of a bronchial tree are typically narrower than more proximal portions. Having the diameter 6328 be relatively consistent throughout the length 6324 can be beneficial, however, for establishing and/or maintaining airway patency. For example, it may be beneficial for a degree of relative hyper-expansion of a wall of an airway region to be greater distally than proximally. This is expected to follow from deployment of a consistent diameter implant in a distally narrowing airway region. Other advantages are also possible. Furthermore, in other embodiments, a counterpart of the diameter 6328 may be inconsistent along a counterpart of the length 6324. For example, a counterpart of the diameter 6328 may increase or decrease along the counterpart of the length 6324. In these cases, an average counterpart diameter 6328 of a counterpart proximal end portion 6302 can be smaller or larger than an average counterpart diameter 6328 of a counterpart distal end portion 6303.

With reference again to FIG. 72, the first apex portions 6318 at the intermediate portion 6304 can define a first helix 6336. Similarly, the second apex portions 6321 at the intermediate portion 6304 can define a second helix 6338. In at least some cases, the longitudinal axis 6301 is an axis of symmetry about which the first and second helixes 6336, 6338 are wound. The implant 6300 can define a first helical band 6340 between the first helix 6336 and the second helix 6338. In the illustrated embodiment, successive turns of the first helical band 6340 are spaced apart from one another along the longitudinal axis 6301 such that the implant 6300 defines a second helical band 6342 intertwined with the first helical band 6340. In at least some cases, an average width of the first helical band 6340 is within a range from 30% to 75% of the average pitch 6326 at the intermediate portion 5604 when the implant 6300 is in the deployed state. As the implant 6300 transitions from the delivery state toward the deployed state or the unconstrained state, the average width of the first helical band 6340 parallel to the longitudinal axis 6301 can decrease and an average width of the second helical band 6342 parallel to the longitudinal axis 6301 can increase. Conversely, as the implant 6300 transitions from the deployed state or the unconstrained state toward the delivery state, the average width of the first helical band 6340 parallel to the longitudinal axis 6301 can increase and the average width of the second helical band 6342 parallel to the longitudinal axis 6301 can decrease.

In some cases, it is useful for the second helical band 6342 to still be present when the implant 6300 is in the delivery state. Stated differently, in these cases, it can be useful for successive turns of the first helical band 6340 to be spaced apart from one another along the longitudinal axis 6301 when the implant 6300 is in the delivery state. This can be useful, for example, to reduce or eliminate overlapping of the wire path 6306 when the implant 6300 is in the delivery state. Overlapping of the wire path 6306 can cause the implant 6300 to be less compact in the delivery state than would otherwise be the case. This can be disadvantageous as it may reduce an ability of the implant 6300 to be delivered intraluminally to more distal airways. In other cases, a counterpart of the second helical band 6342 may be eliminated when a counterpart of the implant 6300 is in a delivery state. Stated differently, in these other cases, successive turns of a counterpart of the first helical band 6340 may be overlapping when the counterpart of the implant 6300 is in the delivery state. The circumferential alignment of features within a counterpart of the first helical band 6340 between successive turns thereof can affect whether a counterpart of the wire path 6306 does or does not overlap in these cases. When the circumferential alignment of these features is such that a counterpart of the wire path 6306 does not overlap, then overlapping a counterpart of the first helical band 6340 when a counterpart of the implant 6300 is in a delivery state may be advantageous. For example, via nesting or interdigitation, this overlapping may allow more longitudinally expansive structures to be present in the same longitudinal space. As discussed below, however, circumferential alignment of features within the first helical band 6340 has other implications which may outweigh, conflict with, or be complementary with this potential advantage.

As shown in FIG. 72, a given three of the first apex points 6319 and the corresponding first apex portions 6320 at respective neighboring turns 6322 of the wire path 6306 at the intermediate portion 6304 can be circumferentially aligned with one another. For example, the given three of the first apex points 6319 and the corresponding first apex portions 6320 can be within 5 degrees or within 10 degrees of circumferential alignment with one another. Furthermore, this circumferential alignment can be present for one, some, or all of the first apex points 6319 and the corresponding first apex portions 6320 at the neighboring turns 6322. The lines 6344 in FIG. 72 indicate this circumferential alignment. In at least some cases, the circumferential alignment in the stated ranges persists as the implant 6300 transitions between the delivery state and the deployed state or between the delivery state and the unconstrained state. Accordingly, the given three of the first apex points 6319 and the corresponding first apex portions 6320 at the respective neighboring turns 6322 of the wire path 6306 at the intermediate portion 6304 can be circumferentially aligned with one another when the implant 6300 is in the delivery state, the deployed state, and the unconstrained state. By way of nonbinding theory, this persistence of circumferential alignment may have certain advantages, such as reducing or eliminating a tendency of the implant 6300 to shift after deployment at a treatment location. Such shifting may increase a foreign body response, increase airway erosion, and/or have other undesirable effects.

In FIG. 72, line segments 6346 represent circumferential spacing between successive apex points among the first and second apex points 6319, 6321 along the wire path 6306 at the intermediate portion 6304. In at least some embodiments, an average of this circumferential spacing is within a range from 35 degrees to 95 degrees, such as from 55 degrees to 65 degrees. As with the circumferential alignment, the average circumferential spacing can persist as the implant 6300 transitions between the delivery state and the deployed state or between the delivery state and the unconstrained state. In at least some cases, the average circumferential spacing between successive apex points among the first and second apex points 6319, 6321 along the wire path 6306 at the intermediate portion 6304 when the implant 6300 is in the delivery state is no more than 5% or no more than 10% different than when the implant 6300 is in the deployed state. Similarly, this average circumferential spacing when the implant 6300 is in the delivery state can be no more than 5% or no more than 10% different than when the implant 6300 is in the unconstrained state. By way of nonbinding theory, this persistence of circumferential spacing may have certain advantages similar to the advantages discussed above with regard to the persistence of circumferential alignment.

FIGS. 73A-74B are diagrams showing different respective subtended angles relevant to the implant 6300. In particular, FIGS. 73A and 73B illustrate a portion of the wire path 6306 corresponding to a given one of the first segments 6330 (corresponding to a given one of the first legs 6314) and a given one of the second segments 6332 (corresponding to a given one of the second legs 6316) at opposite sides of a given one of the first apex points 6319 when the implant 6300 is in the unconstrained state and the delivery state, respectively. Similarly, FIGS. 74A and 74B illustrate a portion of the wire path 6306 corresponding to a given one of the first segments 6330 and a given one of the second segments 6332 at opposite sides of a given one of the second apex points 6321 when the implant 6300 is in the unconstrained state and the delivery state, respectively. As shown in FIG. 73A, a first line 6348 between a pair of the first apex points 6319 neighboring one another along the wire path 6306 subtends a first angle 6350 from an intervening one of the second apex points 6321 along the wire path 6306. FIG. 73A also illustrates a length 6352 of the given first segment 6330 and a length 6354 of the given second segment 6332 at opposite sides of the given first apex point 6319. As shown in FIG. 74A, a second line 6356 between a pair of the second apex points 6321 neighboring one another along the wire path 6306 subtends a second angle 6358 from an intervening one of the first apex points 6319 along the wire path 6306. In at least some cases, one or both of the first and second angles 6350, 6358 are within a range from −20 degrees to 20 degrees (e.g., from −20 degrees to 10 degrees) when the implant 6300 is in the delivery state and within a range from 20 degrees to 90 degrees (e.g., from 40 degrees to 90 degrees) when the implant 6300 is in the deployed state. This angle can be negative when segments of the wire path 6306 at opposite sides of an apex point converge and then diverge as they extend away from the apex point.

An average length 6352 of the first legs 6314 at the intermediate portion 6304 can be different than an average length 6354 of the second legs 6316 at the intermediate portion 6304. For example, the average length 6352 of the first legs 6314 at the intermediate portion 6304 can be greater than (e.g., from 20% to 50% greater than) an average length 6354 of the second legs 6316 at the intermediate portion 6304. Furthermore, a ratio of the average length 6352 of the first legs 6314 at the intermediate portion 6304 to the average length of the second legs 6316 at the intermediate portion 6304 can be greater than a threshold value of n/(n−1) with n being an average number of the first legs 6314 per complete turn 6322 of the wire path about the longitudinal axis at the intermediate portion. For example, the ratio of the average length 6352 of the first legs 6314 at the intermediate portion 6304 to the average length of the second legs 6316 at the intermediate portion 6304 can be within a range from 80% to 99% of the threshold value. This may facilitate avoiding overlap of the wire path 6306 when the implant 6300 is in the delivery state without unduly compromising a degree to which the implant supports an airway region and inhibits invagination of a wall of the airway region.

The implant 6300 can have a surprisingly small airway contact density. In general the amount of force needed to expand an airway region wall is relatively independent of the amount of contact between an implant and the airway region wall. Accordingly, smaller airway contact density corresponds to a need for greater force density. The inventors discovered that airways in a human bronchial tree are capable of withstanding surprisingly high force densities. Accordingly, airway contact density can be reduced without unduly compromising performance. Furthermore, low contact density is expected to have beneficial impacts on maintaining airway patency. For example, low contact density is expected to reduce foreign body response and facilitate mucociliary clearance. Moreover, high force density may actually be beneficial by increasing stability as further discussed below. Airway-to-implant contact density is expected to correspond to the following Equation 1 (Eq. 1):

$$\frac{{A}_{iw}}{{A}_{i}} = \frac{n \cdot ( {l}_{s} + {l}_{l} ) \cdot {d}_{w}}{2 \cdot {d}_{a} \cdot {l}_{p}} \\ {A}_{i} = area supported by a single turn \\ {A}_{iw} = area of a single turn \\ {d}_{w} = diameter of implant \\ {d}_{a} = diameter of airway \\ n = number of implant bends per turn \qquad ( Eq . 1 )$$

In at least some embodiments, the implant 6300 is configured to occupy from 5% to 30%, such as from 5% to 15%, of a total area of the first helical band 6340 when the implant 6300 is in the deployed state.

### VIII. IMPLANT STABILITY

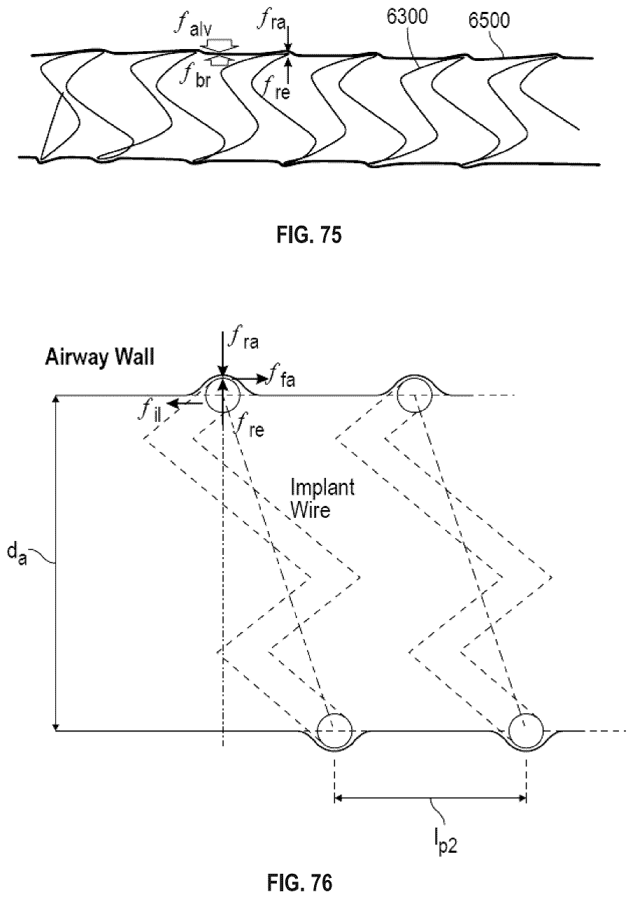

FIG. 75 is a profile view of the implant 6300 in a deployed state within an airway region 6500. In this state, radial forces on the implant 6300 and on the airway region 6500 are expected to be in balance in accordance with the following Equation 2 (Eq. 2):

$${f}_{ra} + {f}_{alv} = {f}_{re} + {f}_{br} \\ {f}_{re} = force of radial expansion of a single wire \\ {f}_{ra} = force of reaction of airway of a single wire \\ {f}_{alv} = force applied by alveolar presure to a single wire turn \\ {f}_{br} = force applied by bronchial presure to a single wire turn \qquad ( Eq . 2 )$$

The diameter 6328 and the radial spring constant of the implant 6300 can be selected in view of the following Equation 3 (Eq. 3):

$${f}_{re} = {k}_{ir} ( {d}_{i n} - {d}_{a} ) = {k}_{ar} ( {d}_{an} - {d}_{a} ) + π \cdot {d}_{a} \cdot {l}_{p} \cdot ( {P}_{alv} - {P}_{br} ) \\ {f}_{re} = force of radial expansion of a single wire \\ {k}_{ir} = spring constant of implant in radial direction \\ {k}_{ar} = spring constant of airway in radial direction \\ {d}_{i n} = nominal diameter of implant \\ {d}_{a} = diameter of airway \\ {d}_{an} = nominal diameter of airway \\ {l}_{p} = implant pitch length \\ {P}_{alv} = pressure in the alveoli \\ {P}_{br} = pressure in the bronchi \qquad ( Eq . 3 )$$

As discussed above, the inventors discovered that airways in a human bronchial tree are capable of withstanding surprisingly high force densities and that high force densities may be beneficial to enhance implant stability and/or for other reasons. Accordingly, the diameter to which the implant 6300 is configured to expand an airway can be many times greater (e.g., at least 2 times, 2.5 time, 3 times, 3.5 times, or 4 times greater) than a nominal diameter of the airway.

Stable contact between an implant and an airway wall can be challenging to achieve for at least two reasons. First, relevant airway regions are typically tortuous, branched, and/or of widely varying diameter. Second, these airway regions typically move significantly and nonuniformly during respiration, coughing, sneezing, etc. Relative movement between an airway region and an implant can cause or contribute to irritation, erosion, foreign body response, and/or other factors that tend to decrease long-term patency. Together with or instead of high force density, the inventors recognized that relatively low resistance to longitudinal deformation together with relatively high resistance to radial deformation can enhance implant stability.

FIG. 76 is a schematic diagram illustrating certain forces and dimensions relevant to implants in accordance with at least some embodiments of the present technology. In FIG. 66, two neighboring turns of an implant 6600 are shown in a deployed state in an airway region 6602. Both radial and longitudinal forces are identified. In at least some cases, when the force of the implant 6600 reacting to elongation/shortening is less than the force of friction on the implant 6600, the implant 6600 tends to remain stable during breathing. The radial and longitudinal spring constants of the implant 6600 can be selected in accordance with the following Equation 4 (Eq. 4):

$$\frac{{k}_{il}}{{k}_{ir}} \leq {μ}_{a - i} \frac{( {d}_{i n} - {d}_{a} )}{( {l}_{pn} - {l}_{p 2} )} \\ {k}_{ir} = spring constant of implant in radial direction \\ {k}_{il} = spring constant of implant in longitudinal direction \\ {μ}_{a - i} = coefficient of friction between airway and spring \\ {d}_{a} = diameter of airway \\ {d}_{i n} = nominal diameter of implant \\ {l}_{pn} = nominal implant pitch \\ {l}_{p 2} = distance between adjacent turns with lung motion \qquad ( Eq . 4 )$$

Implants in accordance with at least some embodiments of the present technology have a ratio of radial spring constant to longitudinal spring constant within a range from 10:1 to 80:1, such as from 15:1 to 80:1 or from 20:1 to 80:1.

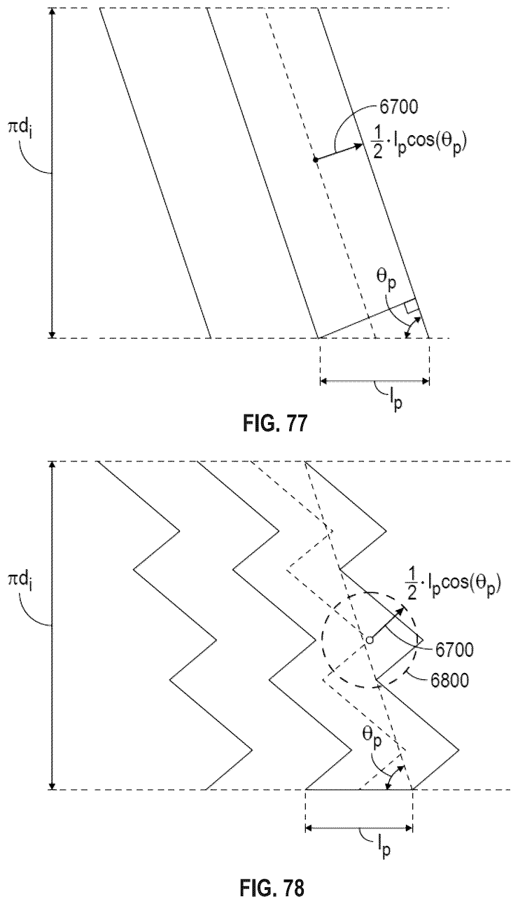

A wire including alternating first and second legs can support and airway to a greater extent than a wire shaped as a simple coil even if both wires have the same pitch. FIG. 77 is a schematic diagram illustrating a maximum distance between a point on an airway wall and a wire path of a simple coil. FIG. 78 is a schematic diagram illustrating a maximum distance between a point on an airway wall and a wire path of an implant in accordance with at least some embodiments of the present technology. The maximum distance in FIG. 77 is represented by line 6700 and can be calculated using the following Equation 5 (Eq. 5):

$$maximum distance = \frac{1}{2} \cdot {l}_{p} cos {θ}_{p} \\ {l}_{p} = implant pitch length \\ {θ}_{p} = implant pitch angle \qquad ( Eq . 5 )$$

In FIG. 78, a circle 6702 having a radius equal to the length of the line 6700 is centered on a point along a line midway between neighboring turns of the wire path. The circle overlaps the wire path indicating that a portion of an airway at the point is closer to the wire and thus better supported with the wire path of FIG. 78 and with the wire path of FIG. 77.

Another implant feature the inventors recognized as potentially relevant to maintaining stable contact between an implant an airway wall during respiration is resistance to flattening from a tubular form toward a more planar form. Some tubular structures have longitudinally distributed substructures (e.g., helical turns) that easily domino or otherwise collapse on one another in response to shear stress parallel to the structures' longitudinal axes. This is problematic because this type of shear stress may occur in airways during respiration. In contrast to blood vessels that expand and contract to a limited extent and primarily radially rather than longitudinally during pulsatile blood flow, airways during respiration expand and contract far more significantly and do so both radially and longitudinally. Accordingly, achieving an adequate resistance to flattening can be far more challenging in the context of pulmonary implants than in the context of vascular implants. Due to the structural features discussed below and/or for other reasons, implants in accordance with at least some embodiments of the present technology are well suited to resisting flattening. For example, implants in accordance with at least some embodiments of the present technology have a ratio of radial spring constant to longitudinal shear modulus suitable for resisting flattening. This ratio, for example, can be within a range from 0.005 to 0.100. In addition or alternatively, implants in accordance with at least some embodiments of the present technology have a ratio of longitudinal spring constant to longitudinal shear modulus suitable for resisting flattening. This ratio, for example, can be within a range from 0.5 to 5.0.

The above and/or other properties that promote stable wall contact during respiration can be related to certain structural features of implants in accordance with at least some embodiments of the present technology. One such feature is the complete or relative absence of stiff bridges between successive helical turns or other longitudinally distributed implant substructures. This feature can promote relatively low resistance to longitudinal deformation together with relatively high resistance to radial deformation, which, as discussed above, tends to promote stable contact between an implant an airway wall during respiration. This feature can also increase the tendency of an implant to flatten from a tubular form toward a more planar form, which, as also discussed above, can have the opposite effect. The inventors discovered, however, that the latter effect can be at least partially mitigated by increasing the average spacing (e.g., pitch) between successive helical turns or other longitudinally distributed implant substructures. Furthermore, both the complete or relative absence of stiff bridges between successive helical turns or other longitudinally distributed implant substructures and the increased spacing between these substructures synergistically help to maintain improved airway patency. Both of these features tend to facilitate mucociliary clearance and/or to reduce foreign body response. Implants in accordance with at least some embodiments of the present technology include longitudinally distributed substructures (e.g., helical turns) within a first helical band extending around a longitudinal axis and define an unobstructed second helical band between windings of the first helical band. In at least some cases, this feature is present together with a ratio of pitch to diameter within a range from 0.3:1 to 1.5:1, such as from 0.5:1 to 1.2:1.

### IX. DELIVERY SYSTEM

An expandable device, such as any of the expandable devices described herein, can be configured for deployment at a treatment location using a delivery system that is navigable through a working channel of a bronchoscope. Although the delivery system is primarily described herein as navigated through a bronchoscope, it should be understood that in some embodiments, the delivery system can be additionally or alternatively navigable through a suitable robotic system (e.g., robotic catheter) or other lumen of a suitable device.

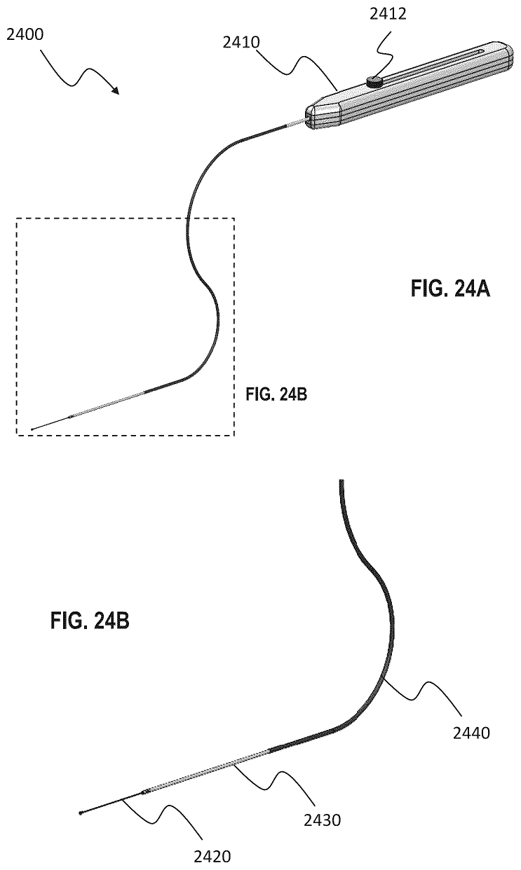

FIG. 24A is an illustrative schematic of a delivery system 2400 configured to deploy an expandable device at a treatment location. As shown in FIG. 24A, the delivery system 2400 includes a handle 2410 and a flexible member portion (also referred to herein as a shaft) that is navigable through a working channel of a bronchoscope. For example, in some embodiments the flexible member portion has an outer diameter of no greater than 3 mm. In some embodiments, the flexible member portion has an outer diameter of no greater than 2 mm. In some embodiments, the flexible member portion has an outer diameter of no greater than about 1.8 mm.

FIG. 24B is a detailed view of the distal portion of the flexible member portion. As shown in FIG. 24B, the flexible member portion can include various members that are telescopically engaged and movable relative to one another. For example, the flexible member portion can include an elongate member 2420 having an implant mounting surface on which the expandable device may be mounted, and an inner sheath 2430 at least partially covering the elongate member 2420. As described in further detail below, the inner sheath 2430 can be movable relative to the elongate member 2420 for selective exposure and/or covering of an expandable device that is mounted on the elongate member 2420. For example, the inner sheath 2430 can be retracted proximally relative to the elongate member 2420 to expose the implant mounting surface and/or an expandable device that may be mounted on the implant mounting surface, thereby enabling deployment of the expandable device. In some embodiments, the inner sheath 2430 can additionally or alternatively be advanced distally relative to the elongate member 2420 to cover the implant mounting surface and/or an expandable device that may be mounted on the implanting surface. The handle 2410 can include an actuator 2412 coupled to the inner sheath 2430 so as to enable a user to selectively retract and/or advance the inner sheath 2430 relative to the elongate member 2420.

As shown in FIG. 24B, the flexible member portion can further include an outer sheath 2440 that at least partially covers the inner sheath 2430. As described in further detail below, the outer sheath 2440 can be configured to engage with a working channel of a bronchoscope. For example, during a procedure for deploying the expandable device in a treatment location, the axial position of the outer sheath 2440 can be fixed to relative to the bronchoscope via a manual lock (e.g., pinching or otherwise holding the outer sheath in place relative to the bronchoscope) and/or a physical locking component (e.g., as described below). In some embodiments, this interaction between the outer sheath 2440 and the bronchoscope can help stabilize the delivery system to the bronchoscope for predictable deployment. However, in some embodiments the outer sheath 2240 may be omitted (e.g., to reduce outer diameter of the flexible member portion).

As described in further detail herein, the flexible member can be navigated toward a treatment location by being advanced through a bronchoscope and/or over a guidewire that has been navigated to the treatment location. Additionally or alternatively, in some embodiments, the flexible member can be actively steerable. Such active steering may, for example, provide additional control of the delivery system in regions of target airways that may be difficult to navigate. Accordingly, an actively steerable flexible member may help enable more accurate placement of an expandable device, and/or otherwise help improve access in certain target airways (e.g., for removal of a placed expandable device). In some embodiments, the flexible member can be actively steered with an actuation system including one or more tethers (e.g., wires, fibers, etc.) that may shape and/or otherwise direct the flexible member in certain directions when activated (e.g., pulled). The tether(s) can, for example, be embedded in a wall of the elongate member 2420, the inner sheath 2430, the outer sheath 2440, between the elongate member 2420 and the inner sheath 2430, and/or between the inner sheath 2430 and the outer sheath 2440.

### A. Handle

The handle of the delivery system functions to enable a user to control the position of the flexible member portion (and the expandable device or implant loaded thereon) inside a patient, from a location outside the patient. In some embodiments, the handle can include a housing that is configured for handheld use, and is coupled to a proximal portion of the flexible member portion. The housing can include suitable features for controlling the flexible member portion, as further described below.

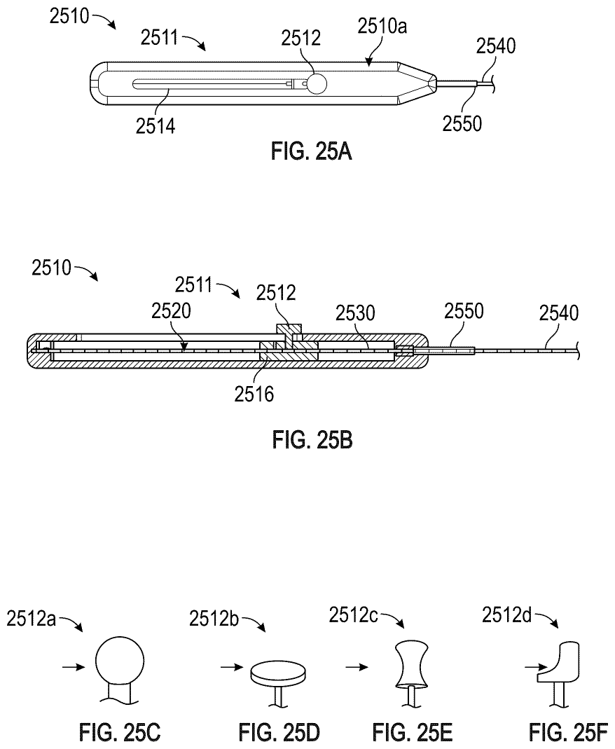

For example, as shown in FIGS. 25A and 25B, a handle 2510 can include a housing 2510a and a sheath actuator 2511 that is operable to control movement of the sheath 2430, which can be similar to inner sheath 2430 shown in FIG. 24B. The sheath actuator 2511 can include a user interface element 2512 coupled to a slider 2516 that is slidably engaged within a track 2514 in the housing 2510a. As shown in FIG. 25B, the slider 2516 can be coupled to the inner sheath 2530 (e.g., via epoxy, welding, fasteners, mechanical interfit, and/or other suitable attachment feature or technique), such that when the slider 2516 moves within the track 2514, the inner sheath 2530 moves in a corresponding manner. As shown in FIGS. 25A and 25B, the track 2514 can be a longitudinal track generally aligned with a longitudinal axis of the handle 2510 and inner sheath 2530, such that proximal movement of the user interface element 2512 along the track 2514 results in proximal movement and retraction of the inner sheath 2530.

As further described below with respect to the elongate member, the handle 2510 can be coupled to a proximal end of the elongate member such that the handle limits the (e.g., fixes) the position and orientation of the elongate member relative to the handle 2510. This coupling can be accomplished, for example, with epoxy, one or more suitable fasteners, and/or the like. For example, the handle housing 2510a (e.g., proximal housing wall) can be coupled to a proximal end of a hypotube 2520. As such, movement of the handle can result in corresponding movement of the elongate member (and the expandable device loaded thereon on the implant mounting surface of the elongate member), such as for positioning of the expandable device within an airway.

Additionally or alternatively, as further described below with respect to the outer sheath, the handle 2510 can be coupled to an outer sheath 2540. The outer sheath 2540 can be coupled to the handle of the delivery device so as to fix the axial position of the outer sheath relative to the handle (and the inner sheath, the elongate member, expandable device, and other components arranged within the outer sheath), but allows the outer sheath 2540 to rotate relative to the handle (and the inner sheath, the elongate member, expandable device, and other components arranged within the outer sheath). Accordingly, coupling the outer sheath to a working channel port of a bronchoscope (as further described below) can advantageously stabilize (e.g., axially secure) the position of an expandable device (loaded on the elongate member relative) to the bronchoscope during a deployment procedure. In some embodiments, the delivery system can also include a strain relief portion 2550 (e.g., reinforcing material, flexure features, etc.) around where the outer sheath 2540 connects to the handle 2510 to help reduce risk of mechanical failure of the delivery system components.

The housing 2510a can be sized and shaped to be held in a hand of a user. For example, the housing 2510a can be generally elongate, and may include an ergonomic shape (e.g., contoured for improving grip stability, contoured for being held in specifically a left hand or a right hand). Additionally or alternatively, the housing 2510a can be any suitable size (e.g., generally smaller for increased portability, and/or lower material costs, generally smaller for being held in a smaller hand, generally larger for being held in a larger hand, etc.). The handle can additionally or alternatively include textural features to improve grip on the handle (e.g., ridges, rings, bumps, high friction materials, etc.). Furthermore, it should be understood that the user interface element 2512 can have any suitable shape. For example, FIGS. 25C-25F depict a variety of examples of user interface elements 2512 that may be considered comfortable to push and/or pull along the handle housing 2510a, such as a bulbous or convex shape (e.g., ball or sphere 2512a), a flattened shape (e.g., disc 2512b), or a contoured shape with one or more concave surfaces for accommodating a thumb or other finger(s) (e.g., pinched shape 2512c or L-shape 2512d). The user interface element 2512 can additionally or alternatively include one or more tactile features to further help a user manually push and/or pull the slider 2516, such as textural features (e.g., ridges, ribs) and/or other features for increasing friction (e.g., rubber or other relatively high friction materials).

In some embodiments, the sheath actuator 2511 can include a suitable intervening gear system between the user interface element 2512 and the slider 2516 that introduces a gear ratio that modifies the travel rate of the slider 2516 relative to the travel rate of the user interface element 2512. The gear ratio can be selected to either increase or decrease the travel distance of the slider 2516 per unit of travel distance of the user interface element 2512 (e.g., gear ratio greater than or lower than 1:1). In some embodiments, the gear ratio can be selected to enable deployment of the expandable device to be accomplished in a selected number of operations (e.g., strokes of a slider, rotations of a wheel or knob, etc.). For example, the gear ratio can be selected to enable deployment of the expandable device to be accomplished with one stroke of a slider in a track (such as any of the slider mechanisms described below). Additionally or alternatively, the gear ratio can be selected to change (e.g., reduce) the amount of force needed to move the user interface element 2512, such as to make it easier to overcome static friction upon initially actuating the user interface element.

In some embodiments, the handle can include a lock that functions to selectively fix an axial and/or rotational position of the inner sheath relative to the elongate member. Such a lock can, for example, help ensure that a user proactively commits to deploying the expandable device by selectively disengaging the lock, and help avoid an inadvertent or premature deployment of the expandable device. Furthermore, when the lock is engaged during shipping or other transit of the delivery device, the lock can help prevent undesirable vibration among components in the delivery system. One example of a lock is shown in FIG. 25B, which illustrates that the lock can be incorporated in the interaction between the user interface element 2512, slider 2516, and the housing 2510. Specifically, the user interface element 2512 can be coupled to the slider 2516 on opposite sides of the housing wall via threads (e.g., user interface element 2512 has a threaded stem that extends through the track opening and engages a threaded hole of the slider 2516, or vice versa). The lock can be engaged by rotating the user interface element 2512 to tighten this threaded engagement, which urges the user interface element 2512 and the slider 2516 closer together on opposite sides of the handle housing wall, thereby fixing the axial location of the slider 2516 in the track and preventing movement of the inner sheath 2530. The lock can be disengaged to enable movement of the inner sheath 2530 relative to the elongate member, by rotating the user interface element 2512 in the opposite direction and loosening the threaded engagement between the user interface element 2512 and the slider 2516 until the slider 2516 can move more freely within the track. In some embodiments, the lock can be repeatedly engaged and disengaged as needed.

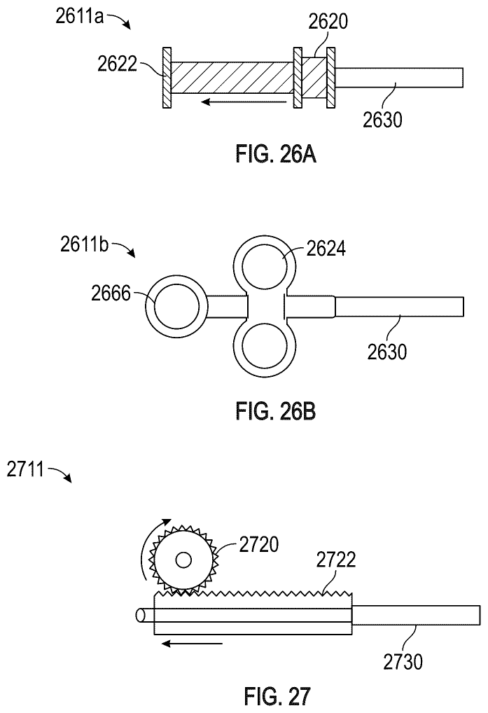

Although FIGS. 25A and 25B depict a slider and track actuator system for actuating the inner sheath, other handle embodiments can include other suitable actuator systems. For example, FIG. 26A is an illustrative schematic of a syringe-based sliding actuator system 2611a including a plunger 2622 and a body 2620 that receives the plunger 2622. The body 2620 can be coupled directly or indirectly to the inner sheath 2630, such that retraction of the body 2620 toward plunger 2622 (or equivalently, actuation of the plunger 2622 into the body 2620) causes the retraction of the inner sheath 2630. Alternatively, the plunger 2622 can be coupled directly or indirectly to the inner sheath 2630, such that retraction of the plunger away from the body causes the retraction of the inner sheath 2630. In some embodiments, other suitable user interface elements can be combined with this mechanism. For example, as shown in FIG. 26B depicting an alternative sliding actuator system 2611b, distal finger holes 2624 can be incorporated in a body coupled to the inner sheath 2630, while proximal finger hole(s) 2666 can be incorporated in a plunger-type mechanism.

In some embodiments, the sheath actuator system for actuating the inner sheath can include a mechanism different from the above-described sliding mechanisms. For example, the sheath actuator system can include a rack and gear mechanism. FIG. 27 is an illustrative schematic of a rack and gear system, including a gear 2720 that is engaged with a rack 2722. The gear 2720 can be coupled to a thumb wheel or other suitable user interface element on the housing (not shown), and the rack 2722 can be coupled to the inner sheath 2730. Rotation of the gear 2720 via the user interface element results in linear movement of the rack 2722, thereby causing linear movement of the inner sheath 2730. In some embodiments, the rack and gear system may include multiple gears with a suitable gear ratio(s) to adjust the travel rate of the rack 2722 (and inner sheath 2730) relative to the rotation rate of the gear 2720 (and user interface element). Similar to that described above with respect to the sheath actuator 2511, the gear ratio(s) may additionally or alternatively be selected to change (e.g., reduce) the amount of required torque on the gear 2720 to move the rack 2722.

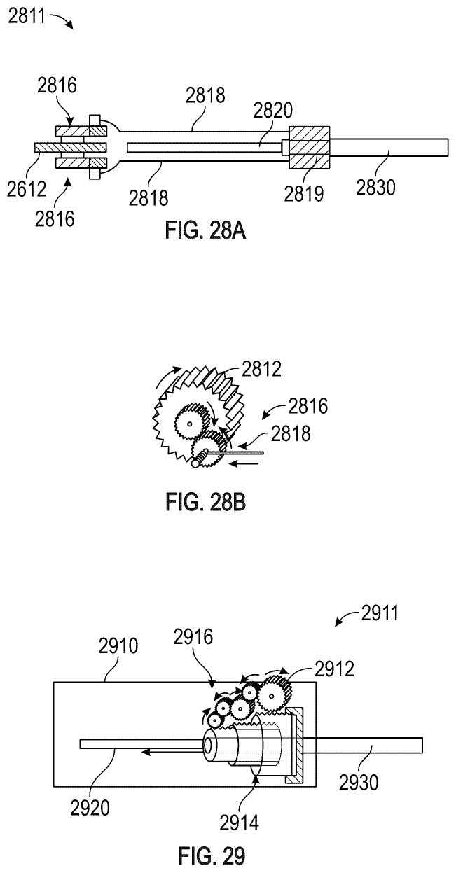

As another example, the sheath actuator system can include a pulley-based system, which can, in some embodiments, reduce the overall length of handle that is required to deploy the expandable device. For example, FIGS. 28A and 28B are illustrative schematics of a pulley system that is actuatable by a user interface element (e.g., wheel 2812) on a handle operable by a user. When the wheel 2812 is turned, it can actuate a gear system 2816 that is coupled via pulley(s) to wind strings 2818. The strings 2818 are coupled to a block 2819 or other suitable anchor to which the inner sheath 2830 is coupled. Accordingly, the turning of wheel 2812 results in strings 2818 winding around the pulley(s) to retract the block 2819 relative to elongate member 2820, thereby causing axial movement (e.g., retraction) of the inner sheath 2830. It should be understood that other pulley-based sheath actuator embodiments can include other suitable gear system arrangements whose details differ from that depicted in the schematic of FIGS. 28A and 28B, but operate on similar principles. For example, the gear system 2816 may include any suitable gear ratio(s).

As yet another example, the sheath actuator system can include telescoping segments in or as part of the handle, which can, in some embodiments, reduce the overall length of handle that is required to deploy the expandable device. For example, FIG. 29 is an illustrative schematic of a handle 2910 including an arrangement with telescoping or nesting segments 2914 that are actuatable by a user interface element (e.g., wheel 2912) on the handle 2910 operable by a user. When the wheel 2912 is turned, it can actuate a gear system 2916 that interacts with one or more of the segments 2914, at least one of which is coupled to the inner sheath 2930. Accordingly, the turning of wheel 2912 results in actuation of the gear system 2914, which extends and/or collapses the combined length of the segments 2914, thereby causing axial movement of the inner sheath 2830 relative to the elongate member 2920. It should be understood that other telescoping segment-based sheath actuator embodiments can include other suitable gear system arrangements whose details differ from that depicted in the schematic of FIG. 29, but operate on similar principles. For example, the gear system 2916 may include any suitable gear ratio(s).

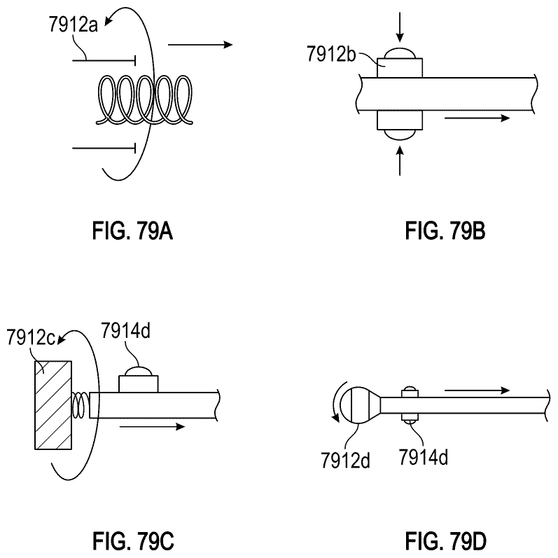

Additionally or alternatively, the sheath actuator system can include any suitable combination of user interface elements. For example, as shown in FIG. 79A, the sheath actuator system can include a user interface element 7912a (e.g., sleeve) that is threaded onto a threaded mount on the handle and coupled to the inner sheath, such that rotation of the user interface element 7912a causes axial movement (e.g., proximal retraction) of the inner sheath. As another example, as shown in FIG. 79B, the sheath actuator system can include a user interface element 7912b that includes one or more push buttons that, when depressed, disengage an axial lock between the inner sheath and the handle and thus enables axial movement (e.g. proximal retraction) of the inner sheath relative to the handle. In some embodiments, the sheath actuator system can include multiple user interface elements, such as a first user interface element to disengage a first axial lock between the sheath and the handle and/or to provide a disengagement force to overcome static friction of the inner sheath, and a second user interface element to control the extent of axial movement of the inner sheath relative to the handle. For example, as shown in FIG. 79C, the sheath actuator system can include a first user interface element 7912c that is threaded onto a threaded mount on the handle and coupled to the inner sheath, and a second user interface element 7914c including one or more push buttons that are operable similar to user interface element 7912b. As another example, as shown in FIG. 79D, the sheath actuator system can include a first user interface element 7912d that includes a rotatable lever or knob and is coupled to the inner sheath, and a second user interface element 7914d that can include a slider operable similar to the slider 2516 (or a push buttons, or other suitable user interface element). It should be understood that the sheath actuator system can include any suitable combination of mechanisms and user interface elements such as those described herein.

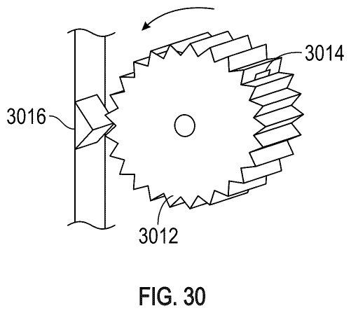

In some embodiments, the handle can include one or more features configured to provide haptic feedback (e.g., tactile and/or audible feedback) that communicates information about deployment status (e.g., deployment rate, distance of inner sheath travel, etc.). For example, in some embodiments the handle may include one or more interfering mechanical components that engage on a periodic basis, such as that shown in FIG. 30. As shown in FIG. 30, the sheath actuator system can include a protrusion 3014 on a moving wheel 3012. The moving wheel 3012 may be turning in a manner corresponding to a user interface element (not shown). For example, the wheel 3012 may share a rotational axis with a wheel-based user interface element on the handle, so as to turn in tandem with user operation of the user interface element, or may share a rotational axis with a gear in a gear system of the sheath actuation system. The protrusion 3014 can periodically encounter mechanical interference with a second protrusion 3016 on another surface adjacent to the wheel 3012, such that contact between the protrusions 3014 and 3016 result in a tactile and/or audible feedback (e.g., added resistance, click, etc.). In some embodiments, the interference can additionally or alternatively trigger emission of a generated sound (e.g., ping, beep, tone, etc.) from a speaker device. The haptic feedback features can be sized and/or shaped to provide suitable feedback at any suitable frequency (e.g., every 0.5 cm or 1 cm of inner sheath retraction/expandable device deployment, etc.). Additionally or alternatively, haptic feedback can be provided in response to information provided by sensors. For example, a light source can be located on one side of wheel 3014, and a light sensor can be located on an opposite side of wheel 3014. The wheel 3014 can include a window such that as the wheel 3014 rotates, the wheel 3014 periodically permits passage of light from the light source to the light sensor, which can detect and track wheel position.

Additionally or alternatively, the handle can be configured to substantially restrict the inner sheath movement in one direction (e.g., in a proximal direction, for retraction of the inner sheath). As such, the handle can help provide better control of deployment of the expandable device, and/or substantially prevent attempts to resheath the expandable device. For example, the sheath actuator system can include a ratchet mechanism that restricts actuation in one direction (e.g., ratchet mechanism attached to a slider, gear, pulley system, etc.).

In various embodiments, the handle may include any sheath actuator not limited to those described herein. It should also be understood that in some embodiments, any of the sheath actuator systems described herein and/or other suitable sheath actuator system can be combined in any suitable manner (e.g., telescoping segments operable by pulleys, slider mechanisms including a gear system, etc.). For example, various embodiments of the handle can include a suitable gear system for assisting reduction of user-provided force for deploying the expandable device (e.g., to overcome static fraction during retraction of the inner sheath).

### B. Elongate Member

As described above, the elongate member in the delivery system functions at least in part to provide structure on which to mount the expandable device (implant) for delivery and placement in a patient. In some embodiments, a first portion (e.g., proximal segment) of the elongate member can have a different structure than a second portion (e.g., distal segment) of the elongate member. Generally, in some embodiments, the elongate member can include an implant mounting surface located on a distal portion of the elongate member for receiving the expandable device thereon.

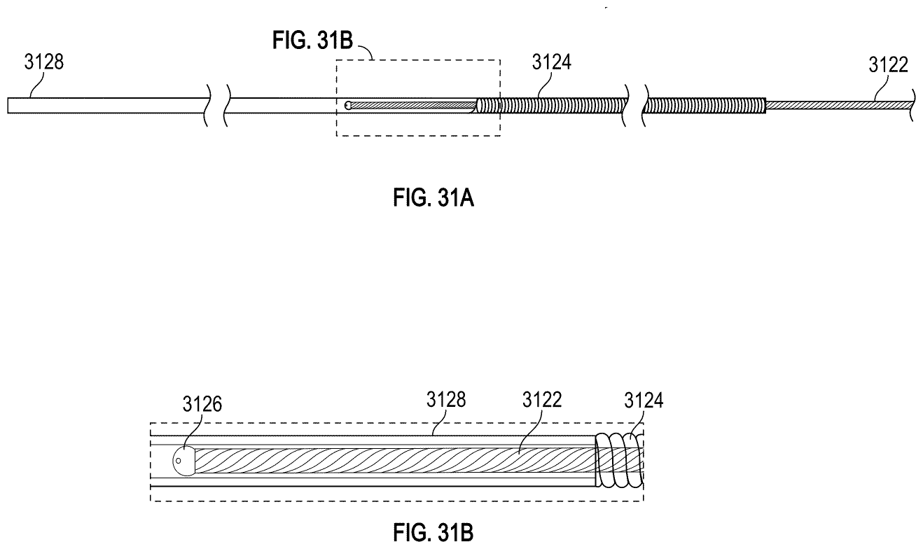

FIG. 31A depicts a portion of an example embodiment of an elongate member 3120. As shown in FIG. 31A, the elongate member 3120 can include a hypotube 3128 that provides structural support for at least a proximal portion of the elongate member 3120. For example, the hypotube 3128 can be coupled to the handle (not shown) and extend towards the distal end of the elongate member 312. The coupling of the hypotube 3128 to the handle limits (e.g., fixes) the position and orientation of the elongate member 3120 relative to the handle. This fixed relationship is shown, for example, in FIG. 25B, which depicts hypotube 2520 coupled to the handle 2510 (e.g., coupling a proximal end of the hypotube to the handle housing 2510, such as with epoxy, one or more suitable mechanical fasteners, etc.).

In some embodiments, as shown in FIGS. 31A and 31B, a distal end of the hypotube 3128 can be coupled (e.g., welded) to a coil 3124. The coil 3124 can have a tight pitch and be sufficiently compacted so as to provide compression resistance for the flexible portion of the delivery system.

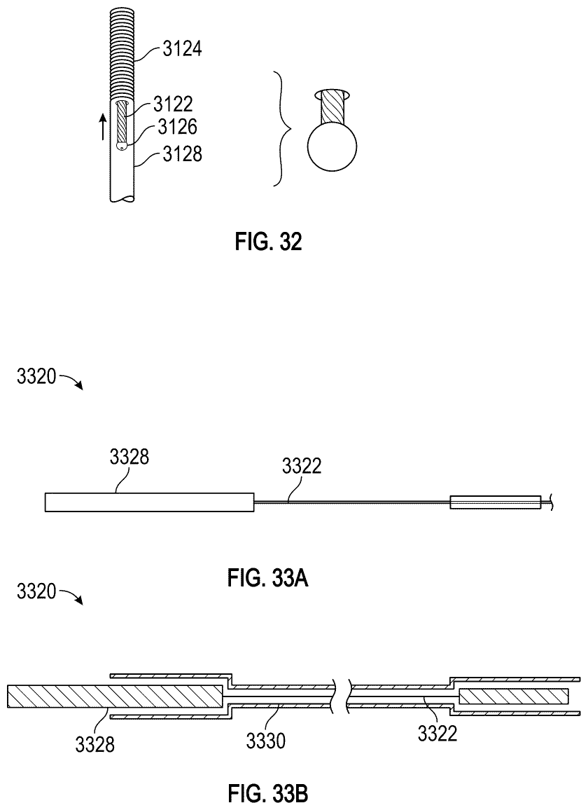

In some embodiments, the elongate member 3120 can further include an inner wire 3122 arranged inside at least a portion of the hypotube 3128 and the coil 3124. The inner wire 3122 can be configured to increase column strength of at least a portion of the elongate member 312. The inner wire 3122 can include, for example, a suitable rope wire. The distal end of the inner wire 3122 can be adjacent to or coupled to an implant mounting surface (further described below). In some embodiments, a proximal end of the inner wire 3122 can terminate in a weld ball 3126 as shown in FIG. 31B. The weld ball 3126 can assist in removal of the delivery system from the patient (e.g., after deployment of the delivery device). For example, the diameter of the weld ball 3126 can be greater than an inner diameter of the coil 3124 such that when the weld ball 3124 contacts the proximal end of the coil 3124, the coil 3124 provides a stop with sufficient mechanical interference to prevent the proximal end of the inner wire 3122 from passing completely through the coil 3124. When the delivery system is being pulled proximally for removal from a patient, proximal movement of the handle may introduce tension that causes the coil 3124 to extend, thereby hampering removal of the delivery system. As shown in FIG. 32, in the event the coil 3124 extends, the weld ball 3126 can abut against the opening of the coil 3124 such that proximal movement of the handle (downwards in the orientation shown in FIG. 32) pulls on the inner wire 3122 instead of on the coil 3124. In other words, this interference restricts the elongation of the coil and pulls on the inner wire 3122 (together with the coil 3124). This limits the maximum length that the overall elongate member can extend in response to proximal withdrawal of the delivery system, thereby assisting removal of the entire delivery system.

The hypotube 3128, coil 3124, inner wire 3122, and/or weld ball 3126 can include, for example, 304SS and/or other suitable materials. In some embodiments, the hypotube 3128, coil 3124, the inner wire 3122, and/or weld ball 3126 can include a material that is radiopaque, so as to enable visualization of the elongate member under fluoroscopy. Dimensions of the elongate member components may vary depending at least in part on the intended application. For example, in some embodiments, as shown in FIG. 32, the relative dimensions of the weld ball 3126 and the inner diameter of the coil 3128 can be sized so as to increase the mechanical interference with each other (e.g., diameter of the coil 3128 can be reduced and/or the diameter of the weld ball 3126 can be increased). Such greater interference may, for example, help reduce failure of the inner wire 3122 to the weld ball 3126 during delivery system removal.

In some embodiments, the coil 3124 may be omitted from the elongate member, which may, for example, help reduce the overall outer diameter of the elongate member and resulting outer diameter of the delivery system (e.g., for use with bronchoscope having a smaller working channel). For example, as shown in FIG. 33A, an elongate member 3320 can include a hypotube 3328 (e.g., similar to hypotube 3128 described above) coupled to an inner wire 3322 (e.g., similar to inner wire 3122 described above), such through welding, where the absence of a coil can reduce the overall outer diameter of the elongate member 3320. As shown in FIG. 33B, in some embodiments without the coil, the delivery device can include an inner sheath 3330 that has a reduced profile around the inner wire 3332, such as with a step down (or taper, etc.) of diameter of the inner sheath 3330. This reduced diameter of the inner sheath 3330 in the region of the wire may help reduce or guard against excessive bowing of the inner wire 3332 (e.g., during advancement of the delivery system, and/or during retraction of the inner sheath relative to the elongate member, each of which may have a tendency to cause the inner wire 3332 to bow under compression).

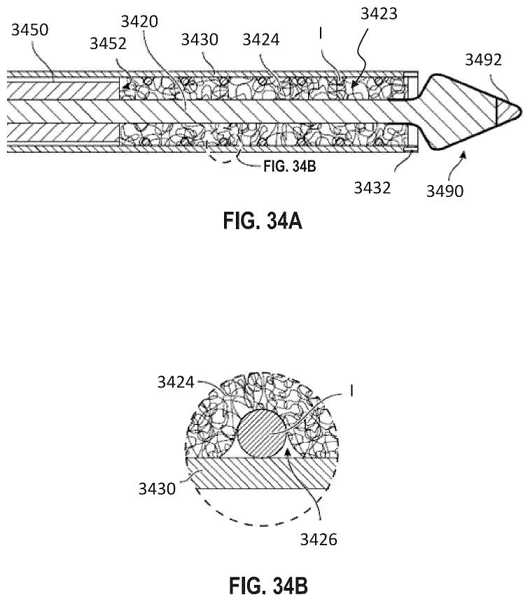

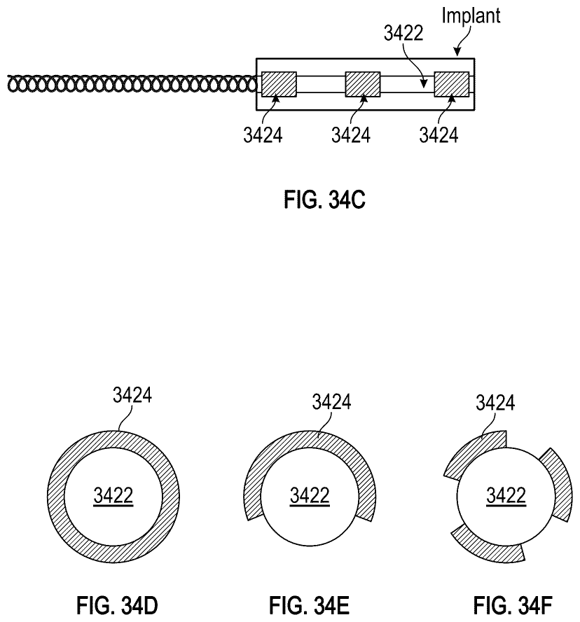

In some embodiments, the elongate member includes an implant mounting surface on which the expandable device is loaded for delivery. For example, as shown in FIG. 34A, the elongate member 3420 can include an implant mounting surface 3423 including a conformable material 3424 configured to adapt to the geometric features of the expandable device I is radially collapsed on the implant mounting surface. The conformable material 3424 can, for example, adapt to one or more interstitial regions of the expandable device I (e.g., open helical region or other open space(s) formed between turns of a wire of the expandable device, between legs of a wire of the expandable device, etc.), such that the conformable material 3424 has an intimate engagement with the expandable device when the device is radially compressed. For example, as shown in FIG. 34B, the conformable material 3424 can form one or more indentations 3426 receiving the wire of the expandable device, which may help the expandable device maintain its axial and/or rotational position on the elongate member 3420. As such, the conformable material enables the expandable device to be “tacked” or otherwise held in place on the elongate member 3420 until the inner sheath 3430 is retracted. In some embodiments, the conformable material may be compressible and/or deformable enough such that the indentations 3426 allow the expandable device I to radially compress enough until its outer diameter is substantially equal to, or less than, the outer diameter of the rest of the implant mounting surface (or rest of the conformable material 3424). For example, when the expandable device is radially compressed and loaded on the conformable material, the indentation(s) 3426 in the conformable material may adapt to a significant portion of the cross-section of the wire of the expandable device (e.g., at least 180 degrees of the profile of a circular wire as shown in FIG. 34B, or at least 150 degrees, or at least 120 degrees). In some embodiments, the implant mounting surface (e.g., the conformable material) can be substantially smooth prior to receiving an expandable device thereon, and adapt to the shape of one or more interstitial regions of the expandable device after the expandable device is radially compressed onto the implant mounting surface.

The conformable material 3424 of the implant mounting surface can have several advantages. For example, the conformable material 3424 allows more tolerance in the rotational and/or axial positioning of the expandable device when the expandable device is being loaded onto the elongate member 3420. Since the conformable material 3424 allows the ultimate placement of the expandable device to be more rotationally and/or axially agnostic within the implant mounting region of the elongate member, the expandable device can be crimped and constrained on the implant mounting surface in a more predictable manner. This results in greater control and consistency in the final radially compressed form of the expandable device on the implant mounting surface, which also results in greater control and predictability in the resulting deployment of the expandable device.

The conformable material 3424 can be, for example, in the form of a pad or coating on the inner wire of the elongate member, or a discrete segment of the elongate member adjacent to the inner wire. In various embodiments, the shape or distribution of the conformable material 3424 can vary in the axial and/or radial dimensions. For example, in some embodiments, the conformable material 3424 can extend along an entire length of an implant mounting surface (e.g., at least as long as the length of the expandable device I), as shown in FIG. 34A. In other embodiments, as shown in FIG. 34C, the elongate member can include multiple segments or sections of conformable material 3424 across the implant mounting surface that are axially spaced apart along a longitudinal axis of the elongate member. Such segments can be equally or unequally distributed along the elongate member. Although FIG. 34C depicts an elongate member with three segments, it should be understood that in other embodiments, the elongate member can include any suitable number (e.g., one, two, three, four, five, or more) of conformable material segments.

Furthermore, in some embodiments, the conformable material 3424 can extend fully circumferentially around the elongate member (e.g., around the inner wire 3422) as shown in the cross-sectional view of FIG. 34D. However, in other embodiments, the conformable material 3424 can be partially circumferential around the elongate member (e.g., around the inner wire 3422). For example, as shown in FIG. 34E, the conformable material 3424 can wrap less than 360 degrees around the circumference of a circular inner wire 3422 (e.g., between about 180 degrees and about 360 degrees, or between about 180 degrees and about 270 degrees, etc.). Additionally or alternatively, in some embodiments, the elongate member can include two or more circumferential segments of conformable material 3424 distributed around the elongate member. For example, as shown in FIG. 34F, the elongate member can include three segments of conformable material 3424 arranged around the circumference of the elongate member. Such multiple circumferential segments can be equally or unequal in arc length, and can be equally or unequally distributed around the elongate member. Although FIG. 34F depicts an elongate member with three partially circumferential segments of conformable material 3424, it should be understood that in other embodiments, the elongate member can include any suitable number (e.g., one, two, three, four, five, or more) of circumferential segments.

The conformable material 3424 can be selected to be sufficiently compressible and/or deformable, yet resilient enough to hold the expandable device in its axial and/or rotational position on the implant mounting surface. In some embodiments, the conformable material 3424 can include a thermoplastic such as Chronoprene®. In some embodiments, the conformable material 3424 can include a flexible extrusion material such as Pebax. The conformable material can, for example, have a durometer of between about 5A and about 75A, between about 15 and about 75A, between about 25A and about 55A, or about 5A, about 15A, about 25A, about 40A, about 55A, or about 75A. Additionally or alternatively, the conformable material can be selected based at least in part on desired radial wall thickness, melting point or flow, adherence properties to the inner wire 3422 and/or expandable device, tensile strength, plastic deformation, elongation, radiopacity, UV stability, biocompatibility, durability under temperature and/or humidity, and/or the like.

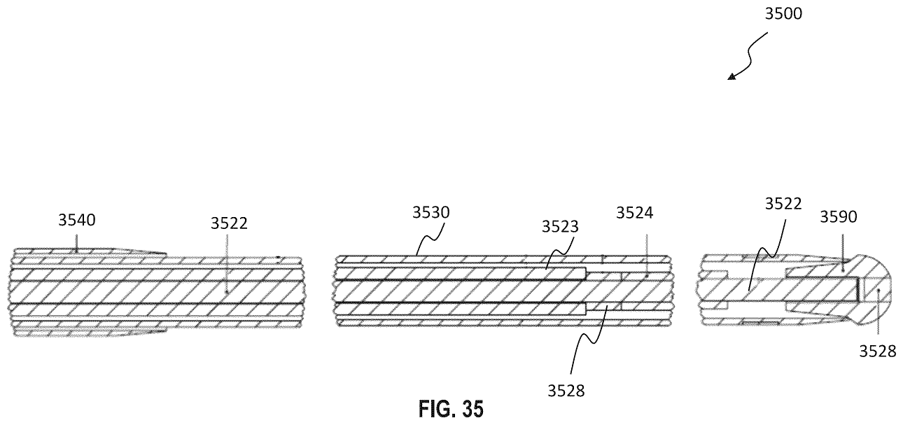

FIG. 35 depicts an illustrative schematic of a portion of another example embodiment of a delivery system 3500 with an implant mounting surface including a conformable material 3524 for receiving an expandable device (not shown). The delivery system includes an outer sheath 3540 and an inner sheath 3530 similar to those described herein, along with an elongate member having an inner wire 3522 arranged within the inner sheath 3530. As shown in FIG. 35, the conformable material 3524 can be arranged over the inner wire 3522, and can be coupled to a coil 3523 (or other suitable portion of the elongate member, such the inner wire 3522 itself or a hypotube), such as with a suitable epoxy 3528. The spatial characteristics of the conformable material 3524 can vary across various embodiments, similar to that described above.

In some embodiments, the implant mounting surface can additionally (e.g., in combination with having a conformable material) or alternatively include other feature(s) for engaging or otherwise securing the expandable device thereon. For example, in some embodiments, the implant mounting surface can include one or more bioadhesives (e.g., a synthetic polymer, a polysaccharide, cellulose, chitosan, fibrin, and/or other suitable bioadhesives, etc.). Additionally or alternatively, in some embodiments, the implant mounting surface can include a textured surface, such as including one or more outward projections (e.g., ribs, bumps, other uneven or non-smooth surface, etc.) and/or a highly frictional material (e.g., an elastomer).

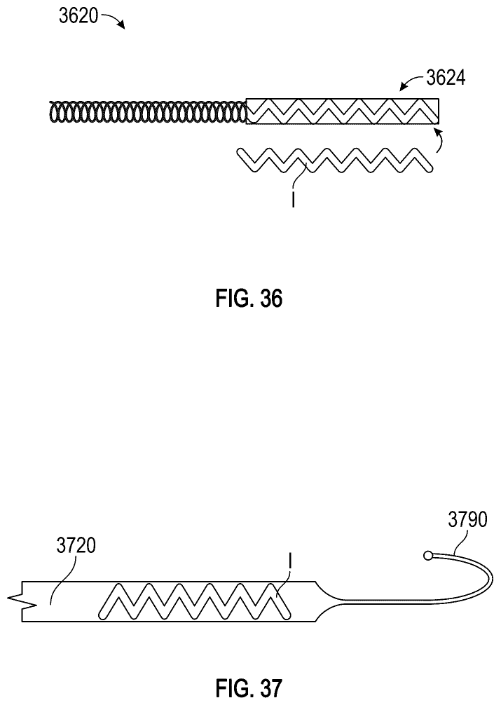

In some embodiments, the implant mounting surface can additionally or alternatively include other features for receiving and positioning an expandable device on the elongate member. For example, the implant mounting surface can have one or more features complementary or corresponding to the overall shape or key geometric points of the expandable device. In these embodiments, the implant mounting surface can include a material that is harder than the expandable device (e.g., rigid or semi-rigid material). For example, FIG. 36 is an illustrative schematic of an example embodiment of an elongate member 3620 with an implant mounting surface including one or more cutouts that correspond to the shape of the radially constrained configuration of the expandable device I. In some embodiments, the cutout 3624 can include a generally helical recess or channel that matches the crimped shape of the expandable device I. Additionally or alternatively, in some embodiments, the implant mounting surface can include radially outwardly projecting bumps or pins that correspond to certain peaks and/or valleys (e.g., vertices) of the expandable device. Such cutouts and/or outwardly projecting features for receiving and positioning the expandable device can, for example, be molded, machined, or otherwise formed in any suitable manner.

As another example, the delivery system can additionally or alternatively include a proximal stop that functions to limit the proximal position of the expandable device I along the elongate member. For example, the delivery system can further include a proximal stop 3450 positioned around the elongate member 3420 and within the inner sheath 3430. The proximal stop 3450 can have a distal-facing surface 3452 configured to abut a proximal end of the device I.

Furthermore, in some embodiments, the delivery system can include an atraumatic tip at the distal end of the elongate member. An atraumatic tip can, for example, help identify the location of pleura during a deployment procedure. For example, as shown in FIG. 35, the elongate member can include a rounded tip 3528, which in some embodiments can be coupled to the inner wire 3522 by epoxy or in another suitable manner. FIG. 34A depicts another example embodiment of an atraumatic tip 3490 that is more tapered than tip 3528. In some embodiments, the atraumatic tip can include one or more radiopaque markers (e.g., marker 3492 shown in FIG. 34A) to help visualize the distal end of the delivery system under fluoroscopy. Such visualization can be helpful, for example, to help avoid inadvertent trauma to the pleura during deployment of the expandable device. As another example, the elongate member can include a soft, guidewire-type tip that is configured to flex upon contact with soft tissue. FIG. 37 is an illustrative schematic of an example of such a guidewire tip 3790 at the distal end of an elongate member 3720, where the guidewire tip 3790 is flexible and/or curved into a rounded shape (e.g., into a hook shape). Additionally or alternatively, to help determine location of pleura, the delivery system can, in some embodiments, be compatible with a separate guidewire (e.g., “over the wire” technique) that is used to separately identify the location of pleura. In some embodiments, use of a guidewire-like tip and/or a separate guidewire can obviate the need for fluoroscopy to track location of the delivery device during a deployment procedure.

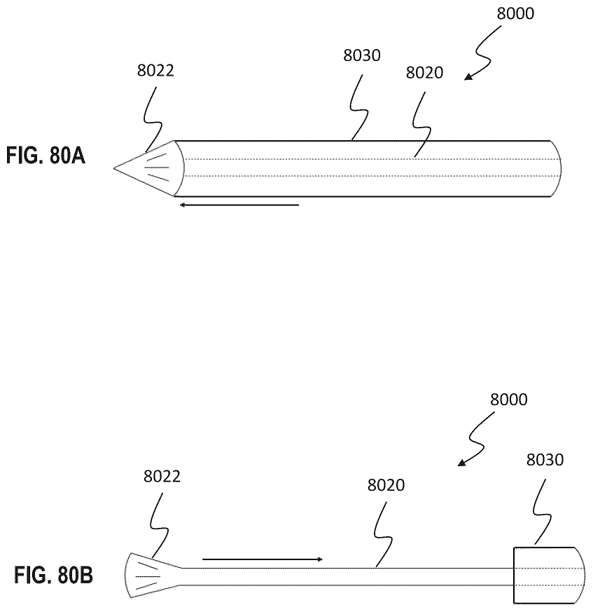

In some embodiments, the elongate member can include one or more features that helps prevent inadvertent engagement of the elongate member with surrounding features (e.g., the expandable device, patient anatomy, outer sheath, bronchoscope, etc.) during withdrawal of the elongate member after the expandable device has been deployed. For example, the elongate member can include a deformable distal end portion that dynamically changes shape to avoid interference with such surrounding features. In some embodiments, the elongate member can include a distal end portion with a first configuration suitable for delivery of the expandable device, and/or a second configuration suitable for retraction of the elongate member after deployment of the expandable device. For example, FIGS. 80A and 80B are illustrative schematic of a distal portion of a delivery system 8000 including an inner sheath 8030 and an elongate member 8020. In FIG. 80A, the inner sheath 8030 is extended over the expandable device (not shown) on the elongate member 8020. The elongate member 8020 can include a deformable tip 8022. In a first configuration as shown in FIG. 80A, the deformable tip 8022 can cover the distal opening of the inner sheath 8030 so as to provide an atraumatic tip for insertion into airways. After the inner sheath 8020 is retracted to deploy the expandable device, the delivery system (including the elongate member 8020) can be withdrawn in a proximal direction to be removed from the patient. During the withdrawal of the delivery system as shown in FIG. 80B, the deformable tip 8022 can transition to a second configuration in which the deformable tip 8022 is in a low profile or other state that is less likely to inadvertently catch on surrounding features as the delivery system is withdrawn. For example, the deformable tip 8022 can invert, fold (e.g., along radial pleats), and/or radially collapse as the delivery system is withdrawn. The deformable tip 8022 can passively transition from the first configuration to the second configuration as it interacts with and reacts to surrounding features, and/or can be actively controlled (e.g., with pull wires or tethers). In some embodiments, the deformable tip 8022 can include a flexible membrane and/or other suitable flexible material.

### C. Inner Sheath

The inner sheath of the delivery system functions to selectively cover and/or constrain the expandable device (implant) loaded on the elongate member. As described above, the inner sheath can be arranged radially over the elongate member, and can be retracted to expose and allow the expandable device to expand (e.g., through self-expansion) to a radially expanded configuration.

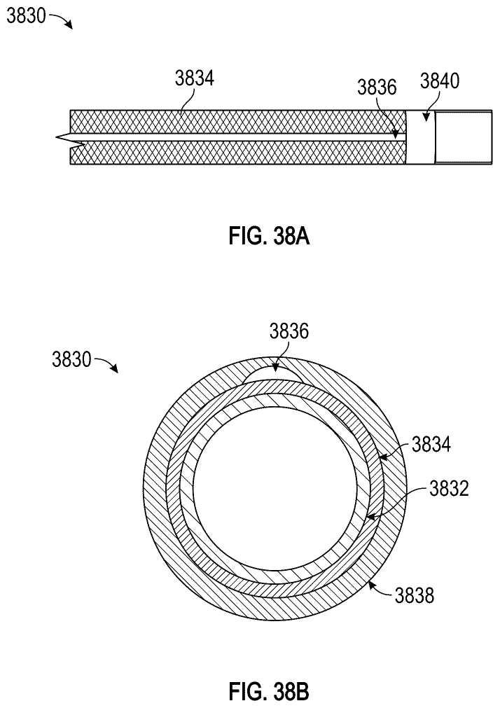

In some embodiments, the inner sheath can include a braided shaft including multiple layers of materials. For example, FIGS. 38A and 38B are illustrative schematics of an example embodiment of an inner sheath 3830 including an inner liner 3832, a braid 3834, and an outer jacket extrusion 3838. The inner liner 3832, which may interface with the elongate member and/or an expandable device in the delivery system, can include a lubricious or low friction material (e.g., PTFE) to help reduce friction when the inner sheath is retracted. The braid 3834 functions to provide structural reinforcement for the inner sheath that maintains suitable flexibility. In some embodiments, the density of braid (e.g., braid pick count) can vary over the length of the inner sheath so as to vary the degree of flexibility along the length of the inner sheath. For example, in some embodiments, the braid pick count can be higher for more distal region(s) of the inner sheath, to allow the delivery device greater overall flexibility at its distal region(s), such to enable better navigation through more tortuous anatomy. In some embodiments, for example, the braid 3834 can include at least a distal braid region having a first pick count and a proximal braid region having a second pick count, where the first pick count is higher than the second pick count such that the distal braid region is more flexible than the proximal braid region. In some embodiments, the braid 3834 can have intermediate braid regions with progressively higher braid pick counts from the proximal braid region to the distal braid region to provide for a gradual transition in flexibility in the inner sheath. However, in some embodiments the braid can have a uniform pick count along its length. The outer jacket extrusion 3838 functions to cover the braid 3834 and provide for a smooth outer surface of the inner sheath. The outer jacket extrusion 3838 can, for example, include a suitable thermoset material (e.g., polyimide, nylon, Pebax, etc.) that maintains suitable flexibility and stiffness for a desired wall thickness. In some embodiments, different segments or portions of the outer jacket extrusion 3838 can include different materials with different durometers, such that the different segments of the outer jacket extrusion 3838 can have different flexibilities. Varied flexibility in the outer jacket extrusion 3838 along its length can, for example, be helpful for introducing greater flexibility in a distal portion of the delivery system (e.g., for navigating smaller and/or more tortuous airways) compared to a more proximal portion of the delivery system. For example, in some embodiments, the outer jacket 3838 can include nylon on its proximal end (to provide a stiffer proximal portion) and a Pebax (e.g., 55D Pebax) on its distal end (to provide a more flexible distal portion).

The inner sheath can, in some embodiments, further include a reinforcement member embedded in the wall of the inner sheath to help reduce longitudinal stretching of the inner sheath. For example, as shown in FIG. 38A, a reinforcement member 3836 can extend along the length of at least a portion of the inner sheath. As shown in FIG. 38B, the reinforcement member 3836 can be located over the braid layer 3834 (e.g., between the braid layer 3834 and the outer jacket extrusion 3838), which may, for example, help keep the braid layer 3834 lie evenly around the inner shaft and reduce overall outer diameter of the inner sheath (and hence the shaft of the delivery system). However, in other embodiments, the reinforcement member 3836 can be at least partially located between the braid layer 3834 and the inner linear 3832 and/or the reinforcement member 3836 can be woven within the braid layer 3834. In some embodiments, the reinforcement member 3836 includes a fiber (e.g., an aramid or para-aramid fiber (e.g., Kevlar®, Technora®, etc.)), and/or a wire (e.g., stainless steel, etc.), although other suitable materials can be selected based on desired resistance to deformation under tension, reinforcement member dimension (e.g., thickness or cross-section dimension), number of reinforcement members, etc.

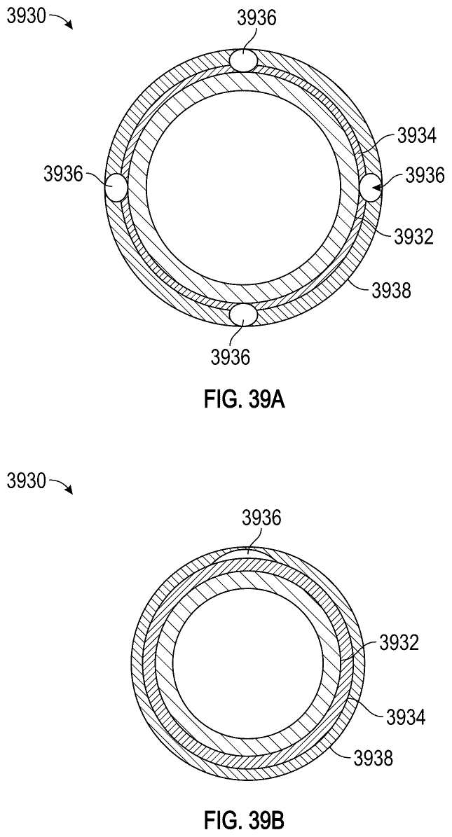

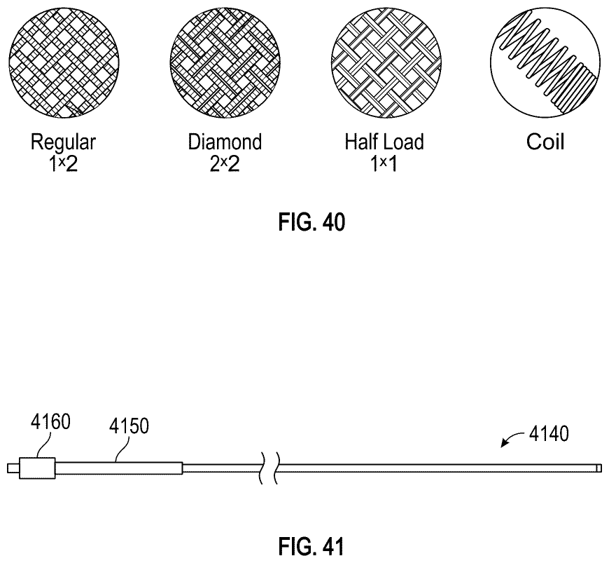

In some embodiments, it can be advantageous to reduce the overall outer diameter of the inner sheath in order to reduce the outer diameter of the delivery system (e.g., to be compatible with certain working channel dimensions of a bronchoscope). Various components of the inner sheath can be modified to accomplish reductions of inner sheath diameter. For example, the thickness of the inner liner, the braid, reinforcement member (e.g., fiber or wire), and/or outer jacket extrusion can be reduced to reduce the overall outer diameter of the inner sheath. For example, use of different braid patterns or replacing the braid with a coil (e.g., as shown in FIG. 40) for the braid layer can result in a reduced thickness of the reinforcement layer between the inner liner and the outer jacket extrusion. Additionally or alternatively, as shown in FIG. 39A, instead of a single reinforcement member 3836 as shown in FIG. 38B, in some embodiments an inner sheath 3930 can include multiple smaller reinforcement members 3936 that collectively enable the inner sheath 3930 to sufficiently resist stretching under tensile load yet with a smaller cross-sectional profile. For example, an inner sheath can include two, three, four, five, six, or more reinforcement members along at least part of its length. Such multiple reinforcement members can be equally distributed around the circumference of the inner sheath (e.g., three reinforcement members arranged circumferentially 120 degrees apart, four reinforcement members arranged circumferentially 90 degrees apart, etc.), which may help retain a balanced cross-sectional profile of the inner sheath in terms of longitudinal stretching resistance. In some embodiments, however, multiple reinforcement members can be unequally distributed around the circumference of the inner sheath (e.g., reinforcement members of different diameters and/or material characteristics that have different resistances to longitudinal stretching can be circumferentially distributed in a way to effectively provide a balanced cross-sectional profile to the inner sheath in terms of longitudinal stretching resistance. FIG. 39B illustrates another example of reducing the overall outer diameter of the delivery system, in which the outer diameter of the elongate member and/or of the implant in its radially compressed configuration can additionally or alternatively be reduced (e.g., the implant can be crimped down to a smaller radius), such that the layers of the inner sheath result in an overall reduced outer diameter of the delivery system. As another example, in some embodiments one or more layers of the inner sheath (e.g., the inner liner) can be thickened to help reduce elongation while omitting reinforcement member(s) (e.g., fiber and/or wire), which may help reduce the outer diameter of the inner sheath while improving the radial symmetry of the inner sheath.

### D. Outer Sheath

The outer sheath of the delivery system functions to provide a surface for engaging with a bronchoscope. In some embodiments, the outer sheath can rotate independently of the inner sheath, which can reduce torquing of the inner sheath and/or elongate member (arranged within the outer sheath) during advancement and navigation of the delivery system through anatomy.

As shown, for example, in FIG. 25A, an outer sheath 2540 can be coupled to the handle 2510. FIG. 41 illustrates a detailed view of an outer sheath 4140 that is similar to outer sheath 2540. Outer sheath 4140 can be coupled to the handle of the delivery device via a rotatable anchor 4160 contained in the housing so as to fix the axial position of the outer sheath 4140 relative to the handle (and the inner sheath, the elongate member, expandable device, and other components arranged within the outer sheath 4140), but allows the outer sheath to rotate relative to the handle (and the inner sheath, the elongate member, expandable device, and other components arranged within the outer sheath 4140). Accordingly, coupling the outer sheath 4140 to a working channel port of a bronchoscope (as further described below) can advantageously stabilize (e.g., axially secure) the position of the elongate member relative to the bronchoscope during a deployment procedure. In some embodiments, the delivery system can further include a strain relief portion 4150 (e.g., reinforcing sleeve, flexure cutouts, etc.) in the region around the connection between the outer sheath 4140 and the handle, to help reduce the risk of failure due to fatigue and/or kinking, etc. as the delivery system is manipulated. In some embodiments, the body wall of the outer sheath can include at least two layers of material, including an inner liner (e.g., PTFE or other lubricious or low friction material) and an outer jacket (e.g., Pebax, Nylon) that provides for a smooth outer surface of the outer sheath 4140.

In some embodiments, the outer sheath can extend along the entire length of the flexible shaft of the delivery system, or can extend along only a portion of the shaft. For example, as shown in FIG. 24B, the outer sheath 2440 can be shorter than the inner sheath 2430.

### E. Guide Sheath

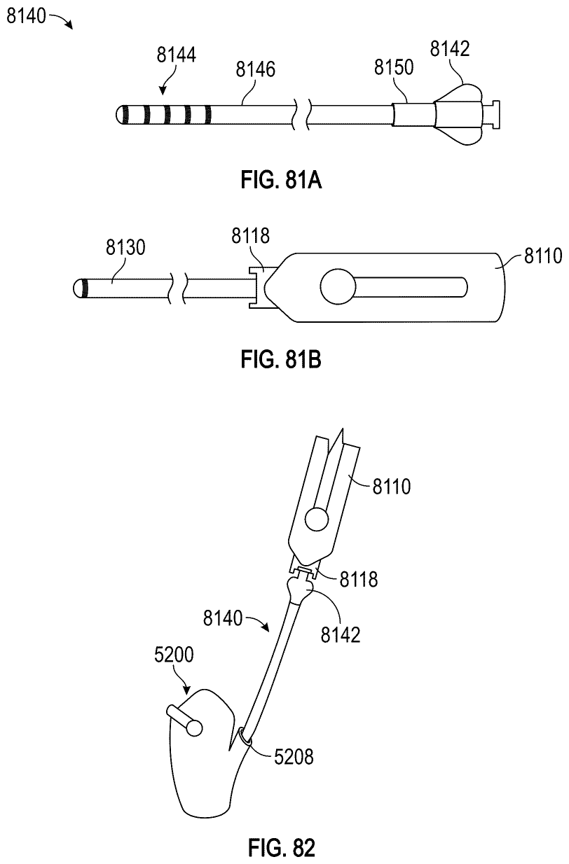

In some embodiments, the outer sheath can be at least initially decoupled and separate from the handle, to function as a guide sheath through which the elongate member and the inner sheath may be introduced. The guide sheath can be similar to the outer sheath described herein, except that the guide sheath can be navigated through target airways towards a treatment location while separated from the handle of the delivery device. For example, FIG. 81A is an illustrative schematic of a guide sheath 8140 including an elongated shaft 8146. Generally, the guide sheath 8140 can be formed in a similar manner to the outer sheath 2540 and/or outer sheath 4140. For example, in some embodiments, the body wall of the guide sheath 8140 can include at least two layers of material, including an inner liner (e.g., PTFE or other lubricious or low friction material) and an outer jacket (e.g., Pebax, Nylon) that provides for a smooth outer surface of the guide sheath 8140. However, in some embodiments, the body wall of the guide sheath 8140 can include a single layer of material (e.g., similar to the outer jacket of the outer sheath). Furthermore, the guide sheath 8140 can include a strain relief portion 8150 (e.g., reinforcing sleeve, flexure cutouts, etc.) in the region around the connector 8142, to help reduce the risk of failure due to fatigue and/or kinking, etc. as the guide sheath 8140 is manipulated. The guide sheath 8140 can include a lumen that is configured to receive a guidewire that may be used to provide more navigational control of the guide sheath 8140 (e.g., to access specific distal airways), and/or at least a portion of the rest of the delivery system, such as an inner sheath 8130 of the delivery system shown in FIG. 81B. In some embodiments, the guide sheath can include a lumen that accommodates (e.g., at different times) both the guidewire and the inner sheath 8130 of the delivery system, or the guide sheath can include different lumens to separately accommodate the inner sheath 8130 and the guidewire.

In some embodiments, the guide sheath 8140 can be selectively coupled to a handle 8110 of the delivery system. For example, when the inner sheath 8130 is advanced into the guide sheath 8140, the guide sheath 8140 can be coupled to the handle 8110 via engagement of a connector 8142 on the guide sheath and a corresponding connector 8118 on or coupled to the handle 8110, as shown in FIG. 82. The connectors 8142 and 8118 can, for example, include a threaded engagement, a snap-fit engagement, and/or other suitable coupling (e.g., Luer lock).

In some embodiments, the guide sheath 8140 can be configured to measure the length of a target airway, which can help inform treatment planning and selection of a proper implant length to be placed in the target airway at a treatment location. For example, the guide sheath 8140 can include one or more markers 8144 (e.g., radiopaque markers, markers visible via bronchoscope camera, etc.) that can be visualized under fluoroscopy to measure the length of a target airway. In some embodiments, the markers 8144 can be equally distributed (e.g., 0.5 cm apart, 1 cm apart, 2 cm apart, etc.) so as to help enable measurement of the target airway within which the guide sheath 8140 is temporarily placed. Additionally or alternatively, at least a portion of the markers 8144 can be spaced apart by distance(s) corresponding to predetermined available lengths of the expandable device to be deployed at the target airway, so as to help enable selection among such predetermined lengths for treatment planning.

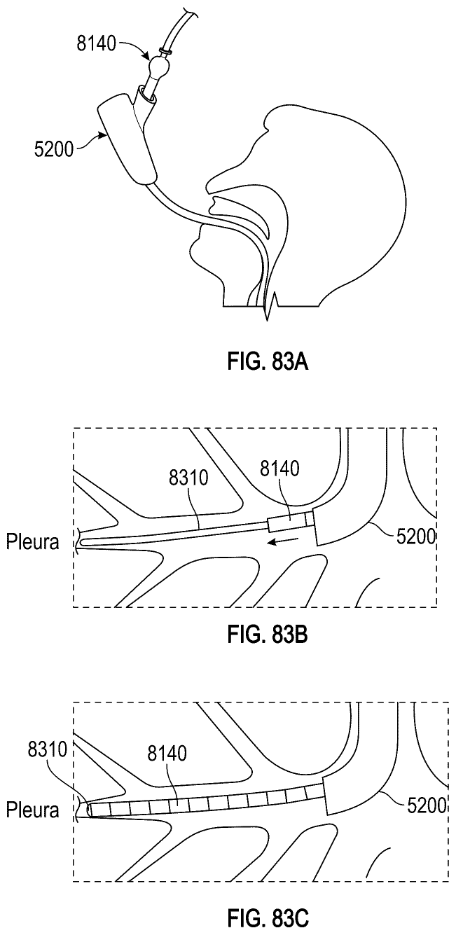

As shown in FIG. 83A, during a procedure for delivering the expandable device, the guide sheath 8140 can be advanced in a working channel of a bronchoscope 5200 (or robotic catheter or other robotic system) in target airways of a patient. In some instances, a guidewire can be advanced distally toward a treatment location and the guide sheath 8140 can be advanced over the guidewire. For example, a guidewire may be advanced distally until it contacts the pleura within a patient (FIG. 83B), and the guide sheath 8140 may be advanced distally fully to the pleura over the guidewire (e.g., 83C). In many instances, the guidewire 8310 and the guide sheath 8140 can extend beyond the distal end of the bronchoscope 5200 such that a number of the radiopaque markers on the guide sheath 8140 may be visualized and used to measure the target airway and/or assist in implant length selection. After the appropriate implant length has been chosen, the guide sheath 8140 can remain such that the distal tip of the guide sheath 8140 remains adjacent to the pleura. The guidewire 8310 can then be removed from the guide sheath 8140, while the guide sheath 8140 remains in place (e.g., held manually, locked to the bronchoscope, etc.).

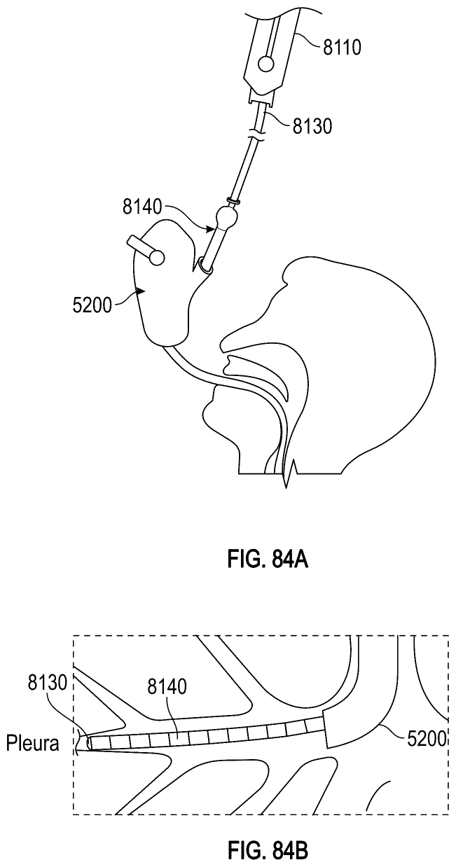

As shown in FIG. 84A, the inner sheath 8130 can be inserted into the guide sheath 8140, and advanced a predetermined or known distance that helps position the expandable device at a desired location relative to the guide sheath 8140. For example, in some embodiments the inner sheath 8130 can be advanced until the distal tip of the inner sheath 8130 is approximately aligned with the distal tip of the guide sheath 8140, as shown in FIG. 84B. Such positioning of the inner sheath 8130 may, for example, help ensure that the inner sheath 8130 (and the expandable device contained in the inner sheath 8130) tracks to the same target airway region that was measured by the guide sheath 8140 for implant selection, as the guide sheath 8140 guides the inner sheath 8130 distally to the pleura.

After the inner sheath 8130 (and the expandable device) are positioned at the target treatment location, the position of the inner sheath 8130 may be fixed relative to the treatment location (e.g., by manually holding the handle 8110, mechanically coupling a proximal region of the inner sheath 8130 to a fixed feature independent of the guide sheath 8140, etc.) to stabilize the position of the expandable device relative to the airway. The guide sheath 8140 can subsequently be retracted proximally such that retraction or other axial movement of the inner sheath results in exposure and deployment of the expandable device without interference from the guide sheath 8140. In some embodiments the guide sheath 8140 can be coupled to the handle 8110 via one or more connectors (e.g., connectors 8142 and 8118), such that proximal movement of the handle 8110 results in proximal movement of the guide sheath 8140. While in this coupled configuration, the guide sheath 8140 can function similar to the outer sheath as described elsewhere herein for deployment of the expandable device. However, in some embodiments, the guide sheath 8140 can be fully proximally withdrawn from the bronchoscope prior to deployment of the expandable device.

In some embodiments, following deployment of the expandable device, the handle 8110 can be withdrawn proximally to remove the inner sheath 8130 and the guide sheath 8140 in tandem from the patient. However, in some embodiments, the guide sheath 8140 can be reused to place an additional expandable device at a second target location. For example, the guide sheath 8140 can be decoupled from the handle 8110 of a first delivery system, the inner sheath 8130 of the first delivery system can be removed from the guide sheath 8140, and the guide sheath 8140 can be navigated to a second target location. When the guide sheath 8140 is positioned at the second target location, the deployment process described above may be repeated to deploy a second expandable device from an inner shaft of a second delivery system. Accordingly, in some embodiments, two or more expandable devices may be positioned and deployed in sequence using the same guide sheath. Alternatively, different guide sheaths may be positioned to help facilitate the positioning and deployment of multiple expandable devices.

### F. Sizing Device

In some embodiments, the delivery system can include a sizing device configured to help determine a suitable length of the implantable expandable device to use. The sizing device can, for example, function to measure the length of a target airway corresponding to a desired treatment location. Generally, the sizing device can include an elongate member with a plurality of markers to help facilitate measuring length of a target airway. In some embodiments, the sizing device can include markers located at a distal portion of the sizing device that is advanced to the target airway, where the markers can be directly aligned with regions of the target airway. Accordingly, when the sizing device is advanced through the bronchoscope to a target location, the depth of its advancement (and the length of the target airway) can be tracked by markers on the distal portion of the sizing device. The elongate member can be flexible so as to facilitate navigation through tortuous airways and/or other anatomy. In some embodiments, a distal tip of the elongate member can be atraumatic (e.g., include a rounded tip, a ball welded to the distal tip, etc.) to help reduce the risk of tissue trauma caused by interaction of the sizing device and tissue.

Additionally or alternatively, the sizing device can include markers located at a proximal portion of the sizing device that can be viewed outside the patient and outside the bronchoscope. For example, the sizing device can be advanced distally until a user feels through tactile feedback that the distal tip of the sizing device touches the pleura, then the sizing device can be retracted proximally a desired amount (e.g., until the distal tip of the sizing device is viewable in a bronchoscopic camera located at the desired location of the proximal end of the expandable device at the target airway). Once the sizing device is retracted by the desired amount, the desired length of the expandable device may be determined based on the markings exposed on the proximal portion of the sizing device.

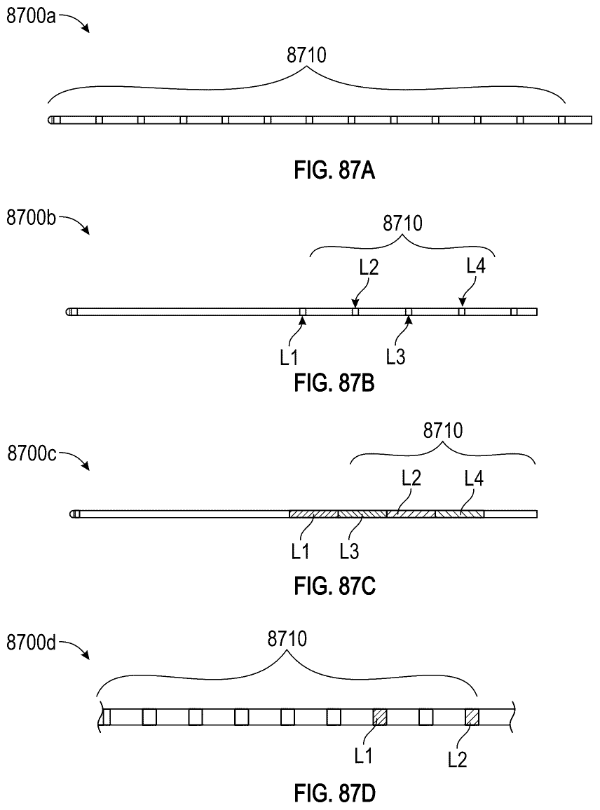

The markers on the sizing device can be configured to be visualized using one or more modalities. For example, in some embodiments, the markers can be radiopaque (e.g., platinum iridium, tungsten) and visualized under fluoroscopy. Markers can be attached in various manners, including but not limited to swaging, crimping, and pad printing, and can be coupled to an exterior and/or interior surface of the sizing device (or embedded in the sizing device). Additionally or alternatively, the markers can be visible from the bronchoscope camera (or camera inserted through a robotic system, etc.) such as under white light imaging, and/or visible by the naked eyes. Additionally or alternatively, in some embodiments, the markers can include discrete segments of the sizing device that are color-coded (or otherwise distinguished by texture, patterning, and/or the like). For example, FIG. 87C illustrates an example sizing device 8700c including color-coded segments 8710 that correspond to different predetermined lengths of the expandable device potentially for placement.

In some embodiments, the markers can be equally spaced apart to provide a ruler measurement, as shown in FIG. 87A illustrating an example sizing device 8700a. Additionally or alternatively, at least a portion of the markers can be located at axial locations corresponding to predetermined potential lengths of the expandable device, as shown in FIGS. 87B-87D (labeled in the figures as “L1”, “L2”, “L3”, “L4”, etc.). In an example embodiment, “L1”, “L2”, “L3”, and “L4” can correspond to expandable device lengths of 55 mm, 70 mm, 85 mm, and 100 mm, respectively, but the markers can alternatively correspond to any suitable predetermined lengths of expandable devices for implantation. In some embodiments, as shown in FIG. 87D, some of the markers 8710 can be equally spaced apart and function to provide a ruler measurement of a target airway, while some of the markers can additionally or alternatively correspond to predetermined potential lengths of the expandable device (labeled as “L1” and “L2” in the example of FIG. 87D).

As described above, in some embodiments the delivery system can include a guide sheath 8140 including markers 8144 such that the guide sheath 8140 functions as a sizing device. The guide sheath 8140 can be advanced to a target airway, and visualization of the markers can help facilitate measurement of the target airway and/or help inform selection of length of the expandable device to be placed at the target airway, as described above.

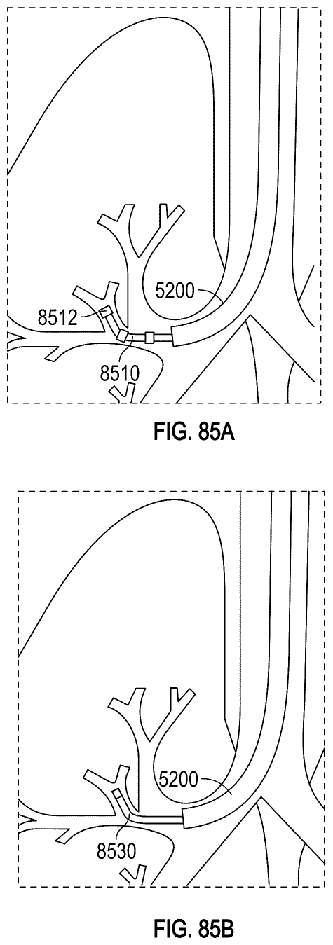

In some embodiments, the sizing device can include a guidewire. For example, as shown in FIG. 85A, a guidewire 8510 can include a plurality of markers 8512 along its length, such as at the distal portion and/or the proximal portion of the guidewire 8510. The guidewire can be advanced through a bronchoscope or robotic system to the target airway, and observation of the position of the markers relative to airway features can be used to measure the target airway length and/or determine suitable length of the expandable device for treatment planning. Subsequently, as shown in FIG. 85B, an inner sheath 8530 (including an expandable device of the desired length) can be advanced over the guidewire to the target location, which ensures that the inner sheath 8530 is in the same location measured by the sizing device guidewire 8510. The guidewire 8510 may be left in place or removed once the inner sheath 8530 and expandable device are at the desired target location and ready for deployment of the expandable device.

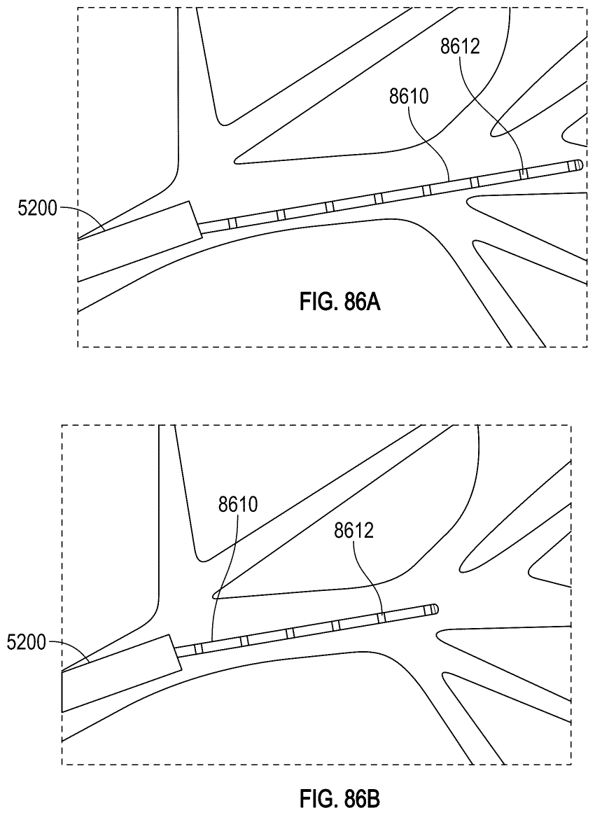

In some embodiments, the sizing device can be a separate elongated member (e.g., probe) that is insertable through the bronchoscope. For example, as shown in FIG. 86A, a probe 8610 or other elongated member can include a plurality of markers 8612 (e.g., marker bands) along itself length, such as at the distal portion and/or the proximal portion of the probe 8610. Like the guidewire 8510 described above, the probe 8610 can be advanced through a bronchoscope or robotic system to the target airway, and observation of the position of the markers relative to airway features can be used to measure the target airway length and/or/determine suitable length of the expandable device for treatment planning. For example, the probe 8610 can be advanced to the target airway until the distal tip of the probe 8610 touches the pleura, then retracted a desired distance corresponding to where the distal end of the expandable device is to be placed (FIG. 86B). Accordingly, once the probe 8610 is positioned at the desired target location, the position of the markers 8610 can be used to identify the appropriate length of the expandable device for the target airway.

In some embodiments, the delivery system can include multiple kinds of sizing devices, whose measurement information can be combined (e.g., averaged or cross-checked in comparison) to improve measurement accuracy and therefore improve treatment planning.

### G. Markers

In some embodiments, the delivery system can include features to facilitate fluoroscopic, bronchoscopic, and/or other visualization during delivery and/or deployment of the expandable device. The elongate member, inner sheath, and/or the outer sheath can include suitable visual markers and/or radiopaque markers (e.g., bands, embedded plug).

For example, in some embodiments, as shown in FIG. 34A, the tip of an elongate member can include a radiopaque marker 3492 (which can made of a material additionally or alternatively intended for visual identification outside of fluoroscopy). As another example, as shown in FIG. 34A, a distal end of the inner sheath can include a marker 3432 (e.g., radiopaque marker and/or visual marker) to facilitate estimating a distal end of the expandable device during delivery and deployment.

In some embodiments, the delivery system can additionally or alternatively include pad printed lines or other visual features (not shown) at an outer surface of the inner sheath. These features can facilitate bronchoscopic visualization. The pad printed lines can be printed in a color that contrasts strongly with the color of the inner sheath (e.g., light colored lines against a dark colored sheath). For example, one line can be aligned with the proximal end of the expandable device to indicate where relative to an airway region the proximal end of the expandable device will be placed after deployment. Furthermore, different indicators can be used to indicate proximal ends of devices of different lengths. For example, one circumferential line can indicate the proximal end of a 70 mm device, two circumferential lines can indicate the proximal end of a 85 mm device, three circumferential line can indicate the proximal end of a 100 mm device, etc.

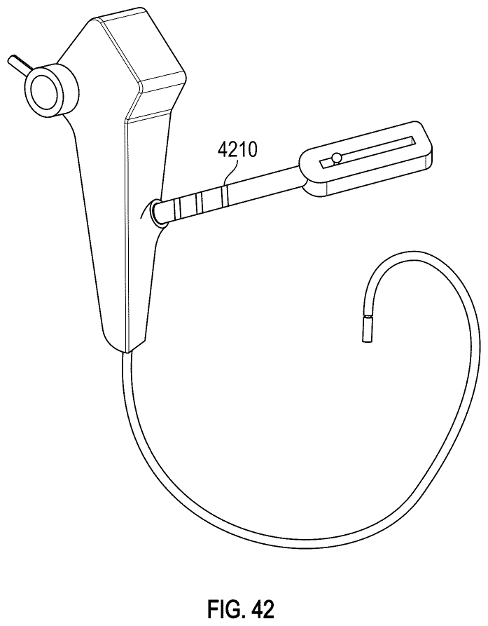

As another example, in some embodiments, a proximal end of the shaft portion of the delivery device can include one or more features to indicate extent of insertion of the delivery device through a bronchoscope working channel. For example, as shown in FIG. 42, a delivery system can include one or more marker bands 4210 (e.g., pad printed bands) that are spaced apart axially along a longitudinal axis of the delivery system shaft, where each marker band indicates a respective insertion depth distance. At least some of the marker bands 4210 can be equally spaced apart (e.g., 1 cm apart), and/or at least some of the marker bands 4210 can be unequally spaced apart (e.g., a series of markers in which adjacent pairs of markers are sequentially 1 cm, 5 cm, and 10 cm apart). Additionally or alternatively, the delivery system can, in some embodiments, include a marker on the shaft that is a location indicating that the tip of the delivery system is aligned with the end of the bronchoscope.

### H. Sensors

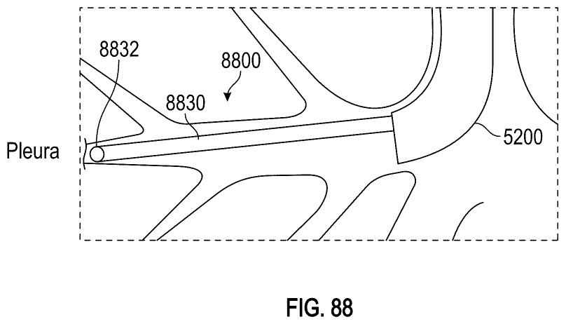

In some embodiments, the delivery system can include one or more sensors that functions to provide information regarding distance between the distal end of the delivery system (e.g., distal end of the outer sheath, inner sheath, or elongate member on which the implant is loaded) and the pleura or chest wall, to help prevent inadvertent tissue trauma as the result of puncture of the pleura during advancement of the delivery system. For example, the sensor may be configured to measure distance between the distal end of the delivery system and the pleura, and communicate this distance information to a user (e.g., a distance measurement, or whether the distal end of the delivery system is within a predetermined distance of the pleura, such as 5 mm, 10 mm, 15 mm, or 20 mm away from the pleura). In some embodiments, as shown in FIG. 88, a delivery system 8800 can include such a sensor 8832 located at the distal end of the delivery system (e.g., distal end of the inner sheath 8830, distal end of the outer sheath, distal end of the elongate member). Additionally or alternatively, such a sensor can be located at the distal end of the implantable, expandable device located in the delivery system.

In some embodiments, the sensor does not require physical contact between the delivery system and the pleura to confirm distance of the pleura, which may advantageously help prevent injury or adverse events that can result from such contact (e.g., infections, irritation, pneumothorax). For example, in some embodiments the sensor can include a proximity sensor, such as an ultrasonic sensor, an infrared sensor, and/or a laser displacement sensor. In some embodiments, sensor information can be transmitted wirelessly (e.g., Bluetooth) or via a wired connection, and can be communicated to a user through a visual modality (e.g., displayed on a monitor display on a console such as robotics system console), an audible modality (e.g., emitted tones or speech indicating distance information), a tactile modality (e.g., haptic feedback communicated through a handle of the delivery system), and/or any suitable manner.

In some embodiments in which the delivery system includes or does not include a distance sensor, other techniques for determining distance between the distal end of the delivery system and the pleura can additionally or alternatively be utilized. For example, the tip of the delivery system (e.g., distal end of the outer sheath, inner sheath, elongate member, guidewire, and/or sizing device) can be distal advanced into an airway until it touches the pleura, then retracted to a desired target location for implant deployment. As another example, the airways and pleura can be visualized through imaging during the implant delivery procedure, which can allow a user to obtain a better view of the delivery system in relation to the pleura. Imaging properties can, for example, be adjusted to improve visualization of the airways and pleura, and/or contrast dyes can be introduced into the patient to improve airway illumination in the imaging (e.g., during fluoroscopy). Additionally or alternatively, other imaging techniques such as cone beam CT can be used to facilitate 3D reconstruction of the patient tissue including the airways and pleura, and sensors, software, and/or delivery system attachments can be used in combination with cone beam CT to assist with treatment location identification, device navigation, device sizing, and/or device placement.

It should be appreciated that other delivery systems are within the scope of the present technology. Moreover, the delivery system can be used with any of the expandable devices disclosed herein.

### X. ENGAGEMENT WITH SCOPE

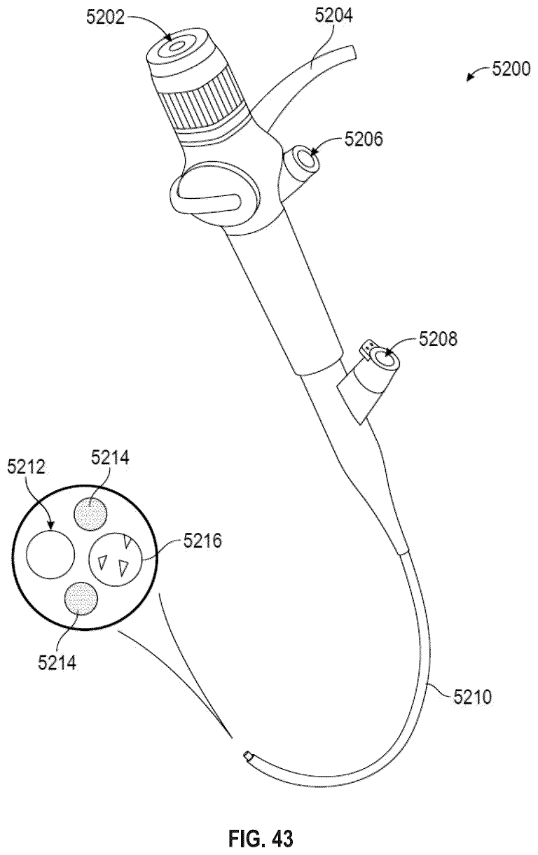

As described above, an expandable device, such as any of the expandable devices described herein, can be configured for delivery through a working channel of a bronchoscope. An example bronchoscope 5200 is shown in FIG. 43. As shown, the bronchoscope 5200 can have a handle with an eyepiece or camera head 5202, a cable 5204 for the light source used for image processing, a suction portion 5206, and a working channel port 5208. The bronchoscope includes an elongated shaft 5210 configured to be advanced down through a trachea to the lungs. The shaft 5210 includes several lumens, including a lumen 5216 supporting a camera or fiberoptic cable bundle, one or more lumens 5214 supporting the light source, and the outlet of the working channel 5212. In some embodiments, the working channel lumen can have a diameter of about 3 mm or less, or about 2 mm or less. In some embodiments of a bronchoscope, a light source and/or camera can be embedded (e.g., a “chip on tip” variation) in addition to or as an alternative to lumen(s) 5214 and/or lumen 5216 for supporting separate light source(s) and a separate camera.

During a deployment procedure, the elongated shaft of a bronchoscope can be advanced through the trachea and bronchial tree (e.g., until the diameter of the elongated shaft approximately matches that of a distended airway and can no longer advance, though the position at which the elongated shaft ceases advancement may be different depending on the bronchoscope being used). For a typical bronchoscope with a 5-6 mm diameter, the stopping point would occur in most patients in the 3rd to 6th generation bronchi. The delivery system can then be advanced distally through the distal opening of the working channel of the bronchoscope until the distal end portion is positioned within a distal portion of the airway (such as, for example, in a terminal bronchiole and/or emphysematous areas of destroyed and/or collapsed airways) near a treatment location, whereupon the expandable device (implant) can be deployed from a delivery system such as any of those described herein.

In some embodiments, during such a deployment procedure, the flexible member portion or shaft of a delivery system can be inserted through the working channel 5212 of a bronchoscope. An outer sheath of the delivery system can be held in place (e.g., manually) relative to the bronchoscope, such as held in place adjacent to the working channel port 5208. Alternatively, in embodiments where the delivery system omits an outer sheath (e.g., to reduce overall outer diameter of the shaft of the delivery system), the handle (or an attachment coupled to the handle) can be directly manually contacted to hold the elongate member (and expandable device mounted thereon) in place relative to the bronchoscope. An expandable device can subsequently be deployed as described elsewhere herein (e.g., advancing the elongate member and expandable device to the treatment location, retracting and/or advancing the inner sheath to expose the expandable device, and allowing the expandable device to transition to a radially expanded configuration). After deployment of the expandable device, the delivery system can be withdrawn from the bronchoscope, and the bronchoscope can also be withdrawn from the patient.

In some embodiments, one or more mechanisms can physically lock the delivery system in place in addition or as an alternative to a user manually holding the delivery system relative to the bronchoscope. In some embodiments, the handle or delivery system shaft can include or be coupled to a first mating component, and the bronchoscope can include or be coupled to a second mating component, where the first and second mating components are configured to mate and selectively lock to one another in order to axially and/or rotationally limit motion of the delivery system relative to the bronchoscope when the lock is engaged.

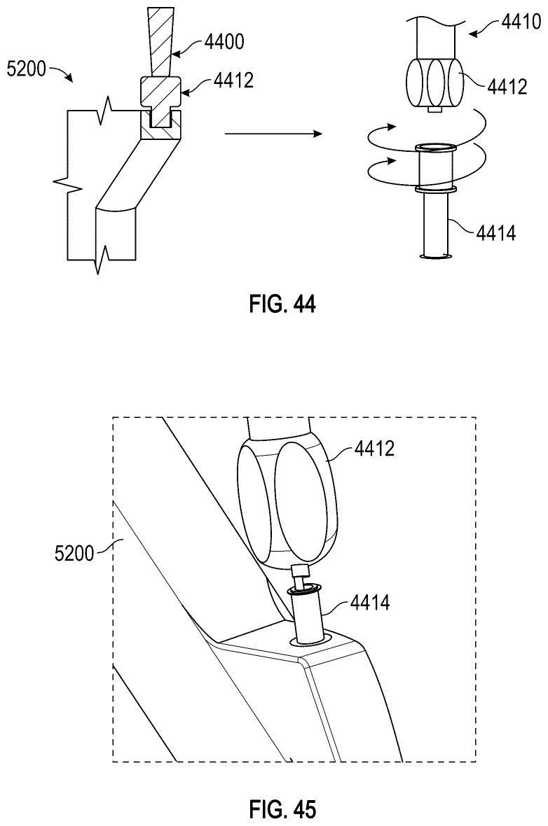

FIGS. 44 and 45 are illustrative schematics of an example of a static lock in which a lock 4410 restricts axial and rotational movement of the delivery system relative to the bronchoscope 5200, when the lock 4410 is engaged. Lock 4410 includes a first component 4412 that is coupled to or integrally formed with the shaft, handle, or other suitable component of the delivery system 4400 and a second component 4414 that is coupled to or integrally formed with the bronchoscope. The first component 4412 can be configured to be threadingly engaged with the second component 4414 in order to couple the delivery system 4400 to the bronchoscope 5200. For example, the lock 4410 can include luer lock components that mate with one another.

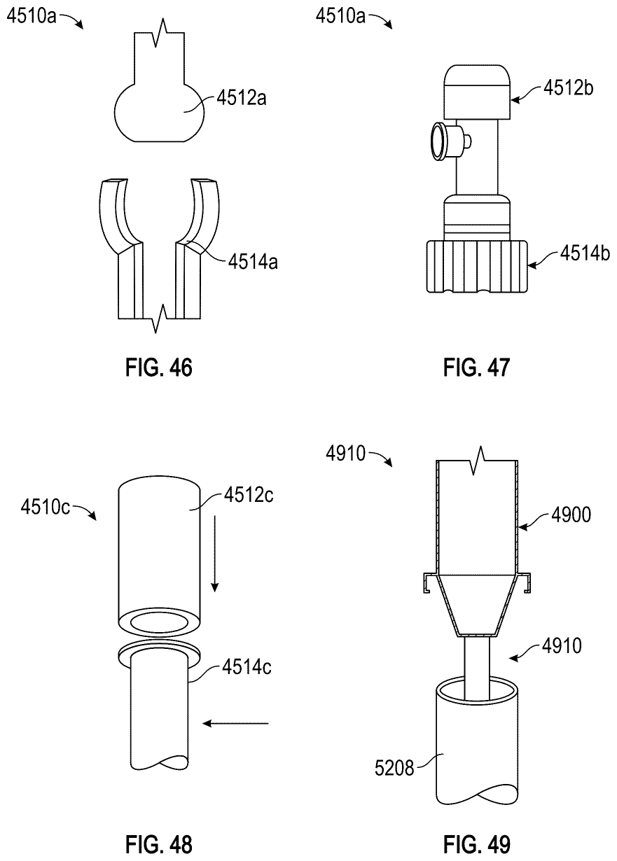

FIG. 46 are illustrative schematics of an example of a lock 4510a similar to lock 4410, except that the first component 4512a (coupled to or integrally formed with the shaft, handle, or other suitable component of the delivery system) and the second component 4514a (coupled to or integrally formed with the bronchoscope), the first and second components 4512a and 4514a engage via a ball and socket connection.

FIG. 47 is an illustrative schematic of an example of a lock 4510b similar to lock 4410, except that the first component 4512b (coupled to or integrally formed with the shaft, handle, or other suitable component of the delivery system) and the second component 4514b (coupled to or integrally formed with the bronchoscope) engage one another with a Tuohy Borst adapter connection.

FIG. 48 is an illustrative schematic of an example of a lock 4510c similar to lock 4410, except that the first component 4512c (coupled to or integrally formed with the shaft, handle, or other suitable component of the delivery system) and the second component 4514c (coupled to or integrally formed with the bronchoscope) engage one another with a biased mechanism. For example, the second component 4514c can be insertable into a receptacle (e.g., sleeve or hole) of the first component 4512c (or vice versa) to engage the first and second components together, and such engagement can be biased (e.g., with a downward force on the first component 4512c as shown in FIG. 48) via a spring or other suitable biasing element, to urge the engagement of the first and second components. In some embodiments, at least one of the first and second components can additionally be laterally movable, such that in order to disengage the lock, the laterally-movable component must be additionally shifted laterally out of alignment with its mating component.

In some embodiments, a mechanism to lock the delivery system in place relative to the bronchoscope can be coupled to or integrally formed with the shaft (or handle or other suitable component of the delivery system) and mate directly with the bronchoscope. For example, as shown in FIG. 49, a plug 4910 can be coupled to or integrally formed with a handle of the delivery system 4900, and couple to the working channel port 5208 of a bronchoscope via mechanical interfit (e.g., snap fit). The plug 4910 can, for example, include an annular lip or rim that grips the wall of the working channel port 5208. Additionally or alternatively, the plug 4910 can include a gasket or O-ring that grips or fits snugly against the inner wall of the working channel port 5208. Alternatively, a similar plug can be coupled to or integrally formed with the working channel port 5208, and couple to the shaft (or handle or other suitable component) of the delivery system 4900 in a similar manner.

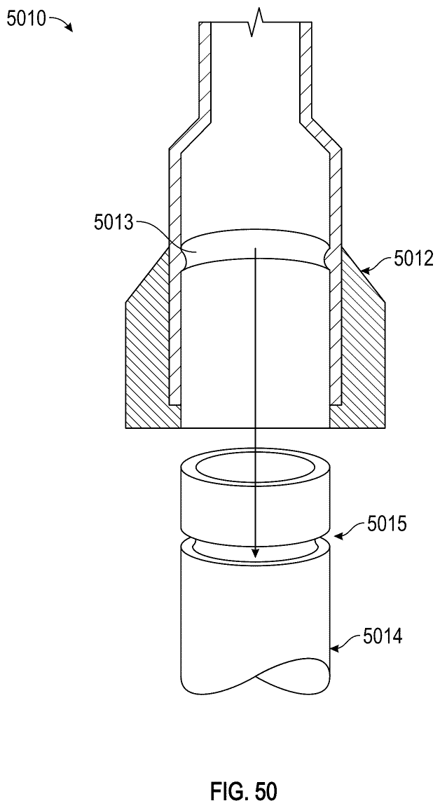

In some embodiments, a lock can be configured to limit axial but permit rotational movement of the delivery system relative to the bronchoscope. For example, as shown in FIG. 50, a lock 5010 can include a mating ring arrangement between a first component 5012 (coupled to or integrally formed with the shaft, handle, or other suitable component of the delivery system) and the second component 5014 (coupled to or integrally formed with the bronchoscope). For example, the first component 5012 can include an outwardly projecting ring 5013 (or ring segments) that is configured to mate with an annular channel 5015 on the second component 5014. Once mated, the ring 5013 can travel within the channel 5015 in that the channel functions as a track, thereby permitting relative rotation between the first component 5012 and the second component 5014, and hence permitting relative rotation between the handle and the bronchoscope. However, the extent of the relative axial motion between the handle and the bronchoscope can be limited by the amount of clearance or tightness of fit between the two components in the axial direction. In some embodiments, the second component 5014 can include an outwardly projecting ring (or ring segments) that is configured to mate with an annular channel of the first component 5012.

Additionally or alternatively, a lock can be configured to limit axial movement of the delivery system relative to the bronchoscope within a certain range of axial movement, to enable “fine tuning” of the axial position of the expandable device loaded in the delivery system. This may be useful, for example, to enable a user to perform some linear adjustments to the position of the expandable device even after locking the delivery device to the bronchoscope. For example, in some embodiments, when the shaft of the delivery system is extended through the working channel of the bronchoscope, a user can view one or more markers on the shaft (e.g., visualization marker bands) through the bronchoscopic camera, where the one or more markers indicates the location of the implant (e.g., proximal end of the implant). Once the delivery system is locked to the bronchoscope, it may be advantageous to allow for the user to adjust, within a certain linear range, the axial position of the elongate member until the one or more visualization markers (and implant) is located at the desired treatment location.

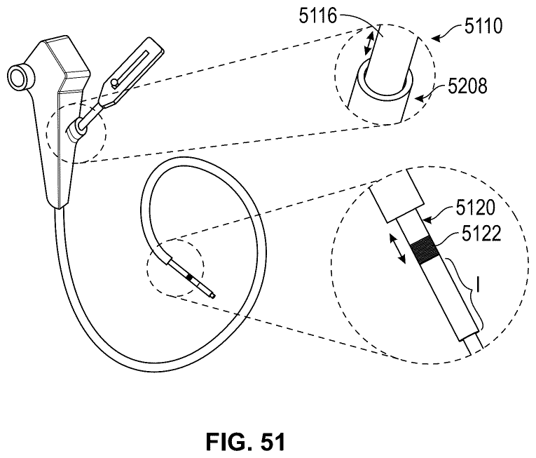

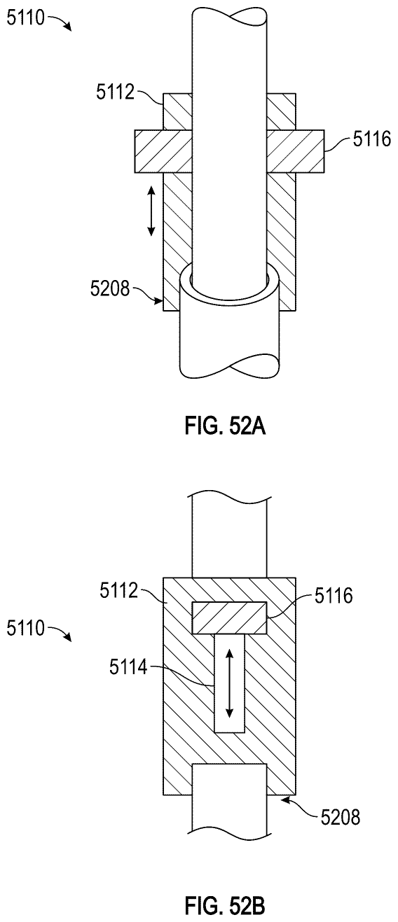

For example, such a lock can include any suitable mating component(s) or feature(s) (e.g., similar to any one or more of the locks described above with respect to FIGS. 44-50), except that the lock can include a slider, knob, or other user interface element that is coupled to the inner sheath of the delivery device and travels within a longitudinal track whose length corresponds to a permissible axial travel adjustment range. As shown in FIGS. 51, 52A, and 52B, the lock 5110 can include, for example, a ball and socket connection arrangement similar to that shown in FIG. 46, except the first component (coupled to or integrally formed with the shaft of the delivery device) includes user interface element 5116 that travels within a track 5114 in the first component. Axial movement of the user interface element 5116 causes axial movement of the delivery device shaft, and accordingly axial adjustment of a visualization marker 5122 on the inner shaft 5120 as viewable through the bronchoscopic camera, which indicates axial adjustment of the location of the implant I. FIGS. 52A and 52B are different side views of the locking arrangement depicted in FIG. 51, with the user interface element 5116 slidable within the track 5114 on the first component 5112. Alternatively, the user interface element 5116 and track 5114 can be located on a component (not shown) that is coupled to or integrally formed with the bronchoscope working channel port 5208.

Although the above coupling arrangements are primarily described with respect to coupling the outer sheath to a bronchoscope, it should be understood that these coupling arrangements may also be included in embodiments in which the outer sheath is a guide sheath that is selectively detachable from the handle of the delivery system. For example, any of the above-described features for enabling fixed and/or adjustable engagement between the outer sheath and the bronchoscope can be incorporated or suitable modified to enable fixed and/or adjustable engagement between a guide sheath and the bronchoscope.

In an additional configuration, a kit or fully-integrated system can include an implant loaded within a delivery system (e.g., as described elsewhere herein) and a single-use, disposable bronchoscope (or robotic system). The bronchoscope can include many of the features described above, including tip articulation (e.g., 90 to 180 degrees) and a working channel (e.g., having a diameter of between about 2.0 mm and 2.8 mm), and can include primarily plastic and polymer components that allow for efficient disposal. The delivery system can be packaged with the disposable bronchoscope or be provided in separate packaging.

In some embodiments, the delivery system (e.g., the inner sheath and/or outer sheath) can be approximately matched in length to the disposable bronchoscope or robotic system, such that when the delivery system is fully inserted into the disposable bronchoscope or robotic catheter, the distal portion of the delivery system can be coupled to the distal portion of the disposable bronchoscope or robotic catheter, and the proximal portion of the delivery system can be coupled to a proximal portion of the disposable bronchoscope or robotic catheter (e.g., at a biopsy port or opening of the working channel). For example, the length of the inner sheath and/or outer sheath can be adapted to the length of the disposable bronchoscope (or robotic catheter) such that the proximal end of the implant is immediately distal to the distal end of the disposable bronchoscope (or robotic catheter). The inner sheath or outer sheath of the delivery system can be coupled via suitable connector (e.g., luer fitting, or other suitable mechanical fastener) to form a connection between the delivery system and the disposable bronchoscope or robotic system during deployment of the implant. Accordingly, the connected delivery system and disposable bronchoscope or robotic system can be manipulated in tandem, thereby allowing a user to operate both components as a single system, which may help improve deployment accuracy and predictability of the expandable device. In some embodiments, the coupler may allow a range of relative axial movement to allow for some adjustment of the implant location relative to the distal end of the disposable bronchoscope or robotic catheter, prior to deployment of the implant. For example, a coupler connecting the delivery system to the disposable bronchoscope or robotic catheter can be similar to any of the couplers described herein with respect to coupling the outer sheath to the bronchoscope (e.g., described with respect to FIGS. 44-52B).

In an additional configuration, a kit or fully-integrated system can include an implant loaded within an inner sheath (e.g., as described elsewhere herein) that is coupled to a handle, and a guide sheath (e.g., as described elsewhere herein) that is selectively and/or removably coupleable to the handle. The guide sheath can be packaged with the inner sheath, handle, and implant, or may be provided in separate packaging. In some embodiments, the kit can further include a single-use, disposable bronchoscope with many of the features described above, including tip articulation (e.g., 90 to 180 degrees) and a working channel (e.g., having a diameter of between about 2.0 mm and 2.8 mm), and can include primarily plastic and polymer components that allow for efficient disposal. The disposable bronchoscope can be packaged with the delivery system with the implant loaded therewithin or may be provided in separate packaging.

### XI. IMPLANT DEPLOYMENT

It should be understood that the techniques described below can be used to deploy any of the expandable devices described herein and/or incorporated by reference, and using any of the delivery systems described herein and/or incorporated by reference.

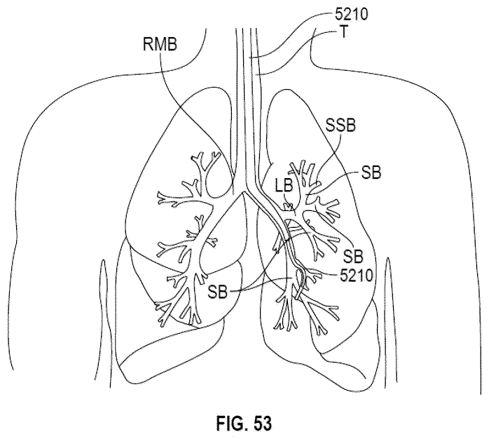

As shown in FIG. 53, the elongated shaft 5210 of the bronchoscope 5200 can be advanced through the trachea and bronchial tree to a desired location, such as where the diameter of the elongated shaft 5210 approximately matches that of a distended airway and can no longer advance. The position at which the elongated shaft 5210 ceases advancement may depend on the bronchoscope being used. For a typical bronchoscope with a 5-6 mm diameter, this would occur in most patients in the 3^{rd} to 6^{th} generation bronchi. The device 4600 can then be deployed in a distal to proximal direction. FIG. 54 shows the device 4600 after deployment.

The distal end portion 4600b of the device 4600 can be placed in a distal airway (e.g., 12^{th} to 15^{th} generation, having a native diameter of 3 mm or less, including in some cases less than 1 mm) with the proximal end portion 4600a of the device 4600 positioned in a proximal airway (e.g., 2^{nd} to 4^{th} generation, having a native diameter of about 4-8 mm). In some embodiments, it may be desirable to position the distal end portion 4600b of the device 4600 no closer than a location between about 1 cm and about 2.5 cm from the chest or pleural wall (e.g., to avoid tissue trauma at the pleural wall). In some embodiments, it may be beneficial to position the proximal end portion 4600a of the device 4600 in a portion of the airway with more cartilaginous tissue (e.g., cartilage-reinforced airways) for better anchoring. The device 4600 and/or wire 4601 can be configured to self-expand to a preset configuration and/or diameter. In some embodiments, the wire 4601 is not heat set and/or configured to self-expand. For example, in some embodiments, the device 4600 and/or wire 4601 is balloon-expandable. In some embodiments, the device 4600 and/or wire 4601 is balloon-expandable and self-expanding.

In some embodiments, the device 4600 can be deployed to a discrete length (e.g., 20, 30, 40, 50, 60 cm, etc.) or, given the axial flexibility of the device 4600, the device 4600 and/or delivery system can be designed for variable length deployment (e.g., each device can be designed to be deployed to up to +/−5 cm of its nominal length) to accommodate variability in patient anatomy. According to some embodiments, the present technology includes multiple devices 4600 delivered in series. The devices placed in series may have different lengths to accommodate and fit different treatment lengths. The multiple devices can overlap, touch, or be spaced apart. If spaced apart, the devices may be spaced no more than a predetermined distance apart in the airway (e.g., 5 mm, 1.0 cm, 1.5 cm, 2.0 cm).

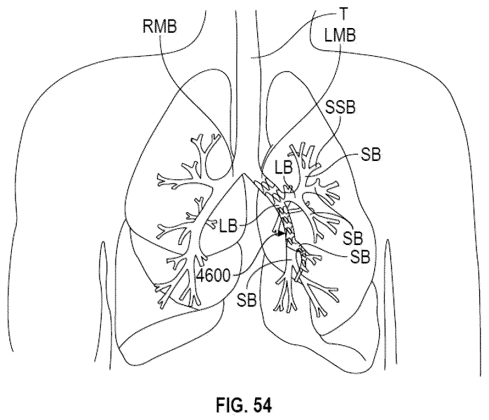

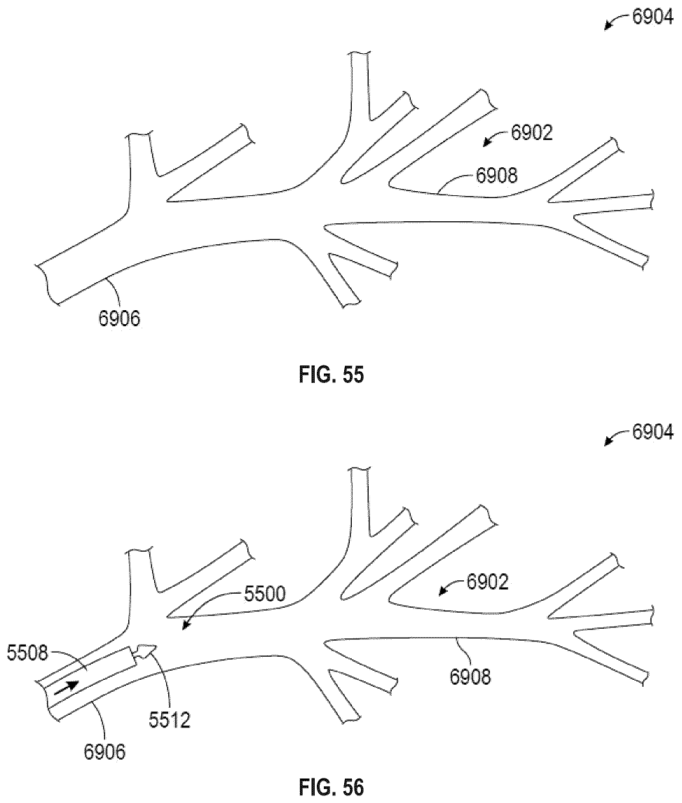

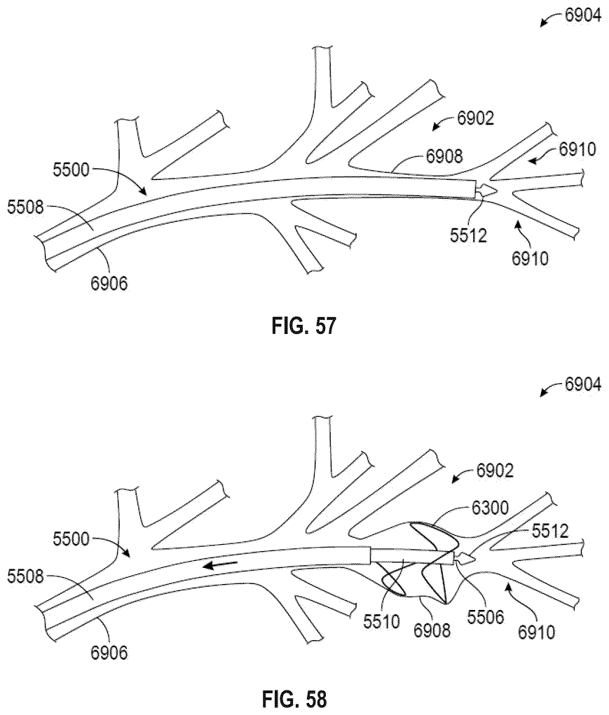

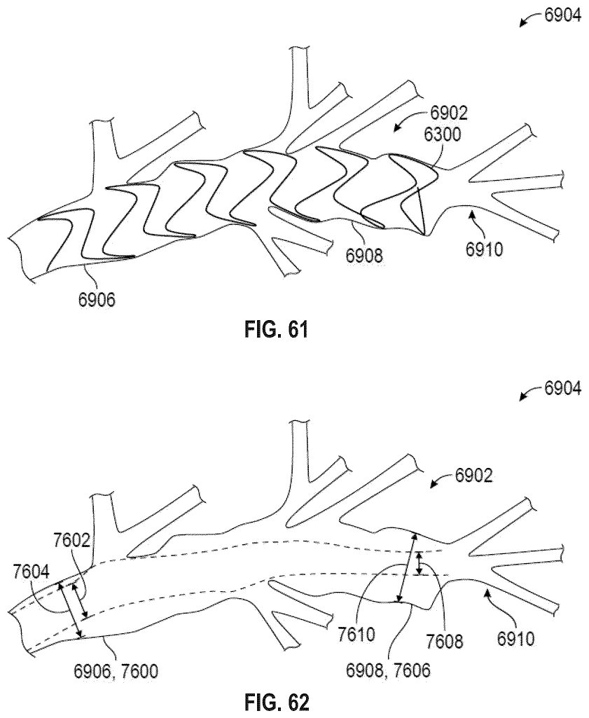

FIG. 55 is an anatomical illustration of an airway region 6902 within a bronchial tree 6904 of a human subject. FIGS. 55-61 are partial schematic illustrations of different respective times during deployment of an implant at the airway region 6902. An implant 6300 can be moved intraluminally within the bronchial tree 6904 toward a treatment location at the airway region 6902. The treatment location can include a first airway 6906 and a second airway 6908 distal to the first airway 6906. A generation of the second airway 6908 can be greater than a generation of the first airway 6906. For example, the generation of the second airway 6908 can be at least 1, 2, 3, 4, 5 or 6 greater than a generation of the first airway 6906. Furthermore, a generation of the first airway 6906 can be at least 3, 4, 5, 6 or an even higher number.

Movement of the implant 6300 toward the treatment location can occur while the implant 6300 is in the low-profile delivery state. For example, the inner sheath 5508 can extend around the implant 6300 and constrain radial expansion of the implant 6300 during this intraluminal movement. As shown in FIGS. 55-57, the delivery system 5500 can be moved distally until the tip 5512 reaches a restriction 6910 (e.g., a bifurcation or trifurcation) of the bronchial tree 6904 too narrow to admit farther distal movement of the delivery system 5500. In some cases, the tip 5512 can expand portions of the airway region 6902 at the restriction 6910. In other cases, the delivery system 5500 is not moved distally far enough to cause this to occur. Interaction between the tip 5512 and the restriction 6910 can be discerned via tactilely (e.g., a clinician may feel resistance when the tip 5512 reaches the restriction 6910), fluoroscopically (e.g., via fluoroscopic imaging of a radiopaque marker (not shown) at the tip 5512), visually (e.g., via an endoscopic camera (not shown) incorporated into the delivery system 5500), and/or in another suitable manner. In some cases, the delivery system 5500 can be deployed via a working channel of a bronchoscope. In these cases, a distal end of the bronchoscope (rather than the tip 5512) may interact with the restriction 6910 to limit a degree to which the implant 6300 can be advanced distally within the bronchial tree 6904. In these cases, a camera of the bronchoscope can be used to guide positioning of the implant 6300.

Once suitably located, the implant 6300 can be transitioned from the delivery state to the expanded deployed state at the treatment location. As shown in FIG. 58, this can include causing relative movement between the implant 6300 and the inner sheath 5508. For example, the inner sheath 5508 can be retracted to expose the implant 6300 progressively beginning with a distalmost portion of the implant 6300 and moving proximally. Exposing the implant 6300 can allow the implant to self expand. For example, exposing the implant 6300 can release at least some resilient bias of the implant 6300 until the implant 6300 assumes an equilibrium state at which outward radial force from the implant 6300 equals inward radial force from the airway region 6902. Alternatively, the implant 6300 can include non-resilient expandable structures configured to expand via a mechanism (e.g., a balloon or other secondary structure within the implant 6300) other than resilience.

During relative movement between the implant 6300 and the inner sheath 5508, a proximal stop in the delivery system can inhibit proximal movement of the overall implant 6300 and the conformable material 5510 on the elongate member 5506 (similar to conformable material 3424 described above with respect to FIG. 34A) can inhibit proximal movement of individual turns of the implant 6300. Thus, the implant 6300 can be deployed in a controlled manner to at least generally retain its longitudinal positioning and configuration as it expands radially. In at least some cases, the length of the implant 6300 is about the same (e.g., no more than 5% or 10% different) immediately after transitioning the implant 6300 relative to while the implant 6300 is still within the inner sheath 5508. Transitioning the implant 6300 can begin with expanding the distal end portion of the implant 6300 at the second airway 6908 (FIG. 58). This can include contacting a wall of the second airway 6908 and an untethered terminus of the wire of the implant 6300 at a portion of the wall of the second airway 6908 proximal to a distalmost end of the implant 6300. Transitioning the implant 6300 can proceed with expanding an intermediate portion of the implant 6300 and then expanding a proximal end portion of the implant 6300 at the first airway 6906. Expanding the proximal end portion at the first airway 6906 can include contacting a wall of the first airway 6906 and an untethered terminus of the wire at a portion of the wall of the first airway 6906 at a proximalmost end of the implant 6300.

In at least some cases, contact between a wall of the airway region 6902 and the implant 6300 simultaneously propagates along different numbers of circumferentially spaced apart portions of the wall during expansion of different portions of the implant 6300. For example, contact between the wall and the implant 6300 can simultaneously propagate along a greater number of circumferentially spaced apart portions of the wall during deployment of the distal end portion 6303 than during deployment of the intermediate portion or during deployment of the proximal end portion. In some examples, contact between the wall and the implant 6300 simultaneously propagates along five or more circumferentially spaced apart portions of the wall during deployment of the distal end portion of the implant 6300 and simultaneously propagates along three or more circumferentially spaced apart portions of the wall during deployment of the intermediate portion and during deployment of the proximal end portion of the implant 6300.

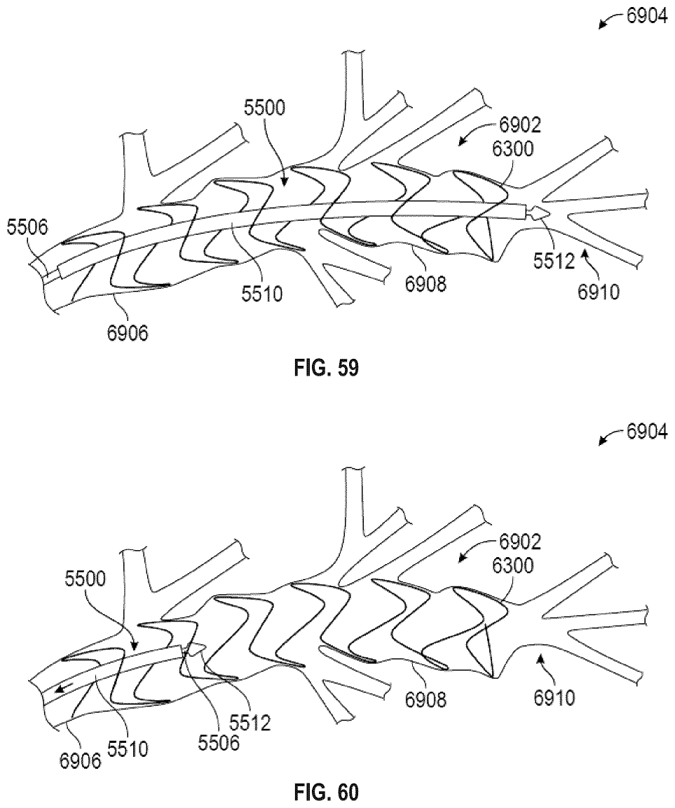

As shown in FIGS. 59-61, transitioning the implant 6300 can free the implant from the conformable material 5510. The conformable material 5510 can then be withdrawn proximally along with other portions of the delivery system 5500, thereby leaving the implant 6300 in the deployed state at the treatment location. Immediately after transitioning the implant 6300, the implant 6300 can exert a force against a wall of the bronchial tree of, for example, at least 0.05 megapascals. The airway region 6902 may be extremely flexible such that transitioning the implant 6300 expands a wall portion of the bronchial tree 6904 coextensive with the length of the implant 6300 well beyond a native diameter of this wall portion. Furthermore, the average diameter of the implant 6300 in the deployed state can be the same as or similar to (e.g., from 70% to 100% or from 80% to 100%) the average diameter of the implant 6300 in the unconstrained state. Additionally or alternatively, a ratio of an average of the diameter of the implant 6300 immediately after transitioning the implant 6300 and the length of the implant 6300 immediately after transitioning the implant 6300 can be within a range from 1:5 to 1:15.

FIG. 62 is an anatomical illustration of the airway region 6902 with certain native and expanded dimensions indicated. This can include expanding a first wall portion 7600 coextensive with a distalmost 10% of the length of the implant 6300 along the longitudinal axis 6301 from a first average native diameter 7602 to a first average expanded diameter 7604 and expanding a second wall portion 7606 coextensive with a proximalmost 10% of the length of the implant 6300 along its longitudinal axis from a second average native diameter 7608 to a second average expanded diameter 7610. In at least some cases, an average expanded diameter at the airway region 6902 throughout the length of the implant 6300 is at least 2, 2.5, 3, or 4 times larger than an average native diameter at this portion of the airway region 6902. In addition or alternatively, a ratio of the first average expanded diameter 7604 to the first average native diameter 7608 can be greater (e.g., at least 4, 6, 8 or 10 times greater) than a ratio of the second average expanded diameter 7610 to the second average native diameter 7608. Furthermore, the first average expanded diameter 7604 can differ from the second average expanded diameter 7610 relatively little, such as between 0% and 20%.

Although deployment of the implant is primarily described as facilitated through proximal retraction of the inner sheath, in some embodiments the movement of at least a portion of the inner sheath in other directions relative to the implant can additionally or alternatively facilitate deployment of the implant beginning from other portions of the implant, which may enable more accurate and/or precise placement of such portions of the implant. For example, deployment of an implant beginning with expansion of a distal end of the implant may enable more accurate and/or precise placement of the distal end of the implant. Deployment of an implant beginning with expansion of a proximal end of the implant may enable more accurate and/or precise placement of the proximal end of the implant. Deployment of an implant beginning with a middle or central region of the implant may enable more accurate and/or precise placement of the middle or central region of the implant.

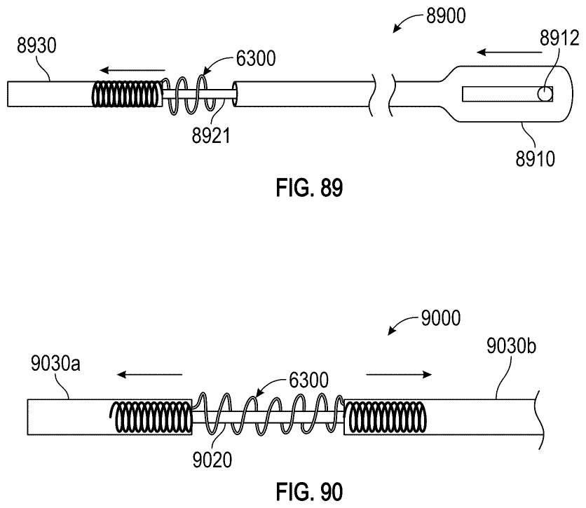

For example, FIG. 89 illustrates an example delivery system 8900 including a handle 8910 and an inner sheath 8930, where at least a portion of the inner sheath 8930 can be distally advanced to expose and enable the implant 6300 to expand from a low-profile state beginning from a proximal end. For example, a user interface element 8912 on the handle 8910 can include a slider operatively coupled to the inner sheath 8930 (e.g., via a push rod 8921) that can be distally advanced or pushed within a slot to cause distal motion of the inner sheath 8930. Other user interface elements such as those described herein can additionally or alternatively be incorporated in the handle 8910 for enabling control of the inner sheath 8930. As another example, the inner sheath constraining the implant 6300 can be everted or eversible to a configuration in which a proximal sheath portion is located within an interior of the implant 6300, and a distal sheath portion is arranged around an exterior of the implant 6300. In this example, the user interface element 8912 can be operatively coupled to proximal portion of the sheath 8930 such that proximal retraction of the user interface element 8912 causes proximal retraction of the proximal portion of the sheath 8930, thereby pulling distally the distal portion of the sheath 8930 in eversion, which exposes and deploys the implant 6300 beginning from a proximal end of the implant 6300.

In some embodiments, the implant can be deployed beginning from a middle or central segment of the implant. For example, FIG. 90 illustrates an example delivery system 9000 including an inner sheath having a distal sheath portion 9030a and a proximal sheath portion 9030b. The distal and proximal sheath portions 9030a, 9030b can collectively constrain an implant 6300 in a low-profile state while the delivery system is advanced toward a target airway. To deploy the implant 6300, the distal sheath portion 9030a can be advanced distally and the proximal sheath portion 9030b can be retracted proximally to expose and enable the implant 6300 to expand beginning from a middle segment of the implant. The distal sheath portion 9030a can, for example, be advanced using any of the sheath actuation systems described above with respect to the delivery system 8900, while the proximal sheath 9030a can be retracted using any of the sheath actuation systems described herein for proximally retracting the inner sheath. Actuation of the distal sheath portion and the proximal sheath portion can occur substantially simultaneously and/or at different times (e.g., sequentially).

### XII. IMPROVING PULMONARY FUNCTION

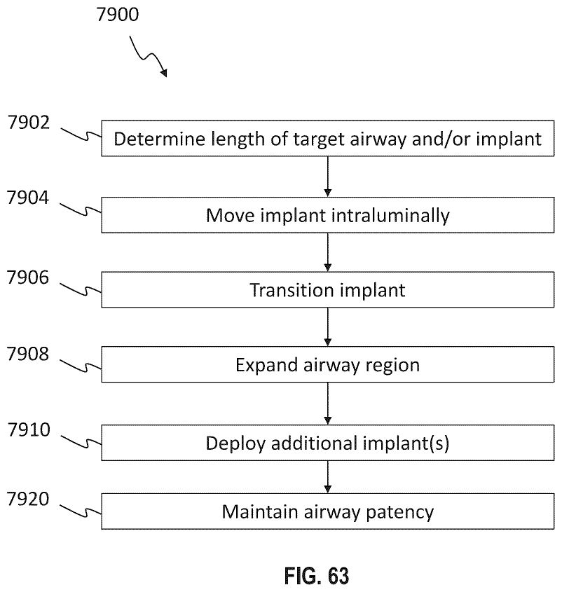

FIG. 63 is a block diagram showing a method 7900 for improving pulmonary function in a human subject in accordance with at least some embodiments of the present technology. In at least some cases, the subject is diagnosed with chronic obstructive pulmonary disorder. As shown in FIG. 63, the method 7900 can include determining a length of a target airway and/or implant (block 7902) and moving an implant intraluminally within a bronchial tree of the subject toward a treatment location within the bronchial tree while the implant is in a low-profile delivery state (block 7904), transitioning the implant from the delivery state to an expanded deployed state at the treatment location (block 7906) and expanding an airway region at the treatment location (block 7908). Aspects of the method 7900 are discussed in detail above in connection with various embodiments of the delivery system (including engagement of an outer sheath with a bronchoscope, operation of a guide sheath, and operation of a sizing device) and implant deployment. The method 7900 can further include deploying additional implants (block 7910). For example, the deployment process described above with reference to FIGS. 55-61 can be repeated with additional implants at different respective airway regions. These airway regions, for example, can be associated with different pulmonary bullae. Deployment of the initial and subsequent implants can release trapped air and reduce or prevent further trapping of air at these pulmonary bullae.

Although not shown in FIG. 63, the method 7900 in some cases can include further modifying the airway region at which a given implant is deployed after deployment of the implant. When a treatment includes deploying multiple implants, this further modification can occur at one, some, or all of the treatment locations. As discussed above with reference to FIGS. 55-61, deploying the implant can expand a wall of an airway region to a first average expanded diameter. Further modification can include subsequently further expanding the wall to a second average expanded diameter larger than the first average expanded diameter. For example, a balloon can be advanced intraluminally to the treatment location with the implant or after the implant is deployed and the delivery system removed. At the treatment location, the balloon can be expanded to cause both the wall and the implant to expand to the larger second average expanded diameter. In at least some cases, the second average expanded diameter is greater than an average unconstrained diameter of the implant. Thus, the balloon can be used to hyper-expand the implant. This can be useful, for example, to create and/or enlarge broncho fenestrations in the wall. As discussed elsewhere in this disclosure, broncho fenestrations may be therapeutically beneficial to release trapped air, to improve airway patency, and/or for one or more other reasons.

In at least some cases, deployment of a first implant can release a first volume of trapped air, placement of a second implant can release a second volume of trapped air, placement of a third implant can release a third volume of trapped air, etc. Implants can be deployed until a sufficient amount of trapped air is released and a sufficient degree of lung volume reduction is achieved for effective treatment of COPD. In some cases, deploying one implant may be sufficient. In other cases, 2, 3, 4, 5, 6, or even greater numbers of implants may be deployed. Furthermore, one, two or another suitable first quantity of implants may be deployed at one time and one, two or another suitable second quantity of implants may be deployed at a second time hours, days, months or even longer after the first time. In a particular example, a first quantity of implants is deployed, followed by gathering monitoring, testing, and/or patient-reported information during a test period, and then a second quantity of implants is deployed based on a degree to which the first quantity of implants was effective in treating COPD symptoms according to the information. In yet another example, additional implants may be deployed occasionally as COPD progresses and new pulmonary bullae develop over many months or years.

Deploying an implant at a treatment location can cause the treatment location to go from being low patency or nonpatent to having therapeutically effective patency. In at least some cases, a portion of the bronchial tree distal to the treatment location is emphysematous and has collateral ventilation. In these and other cases, deploying one or more implants can increase one-second forced expiratory volume by at least 5% (e.g., at least 10%). The method 7900 can further include maintaining airway patency (block 7920). With reference to FIGS. 55-63 together, the method 6300 can include maintaining a therapeutically effective increase in patency at the treatment location throughout a continuous maintenance period while the implant 6300 is in the deployed state at the treatment location. The maintenance period can be at least 3 months, 6 months, 9 months, or another suitable period. During the maintenance period, a first area of a wall portion of the bronchial tree 6904 coextensive with the length of the implant 6300 along its longitudinal axis can be in direct contact with the implant 6300 and a second area of the wall portion can be out of direct contact with the implant 6300. The second area can be at least 5, 8, 10, 12, 14 or more times larger than the first area. In addition or alternatively, the wire of the implant 6300 can occupy from 5% to 30% (e.g., from 5% to 15%) of a total area of the first helical band 6340 during the maintenance period. Furthermore, a maximum invagination of the wall portion at the second area can be no more than 50% of the average expanded diameter of the implant 6300 during the maintenance period. Maintaining airway patency can also include maintaining a mucociliary clearance region at the treatment location substantially free of granulation tissue and mucoid impaction throughout the maintenance period. In addition or alternatively, maintaining airway patency includes maintaining the mucociliary clearance region substantially free of one some or all of inflammation, inflammatory cells, granulation tissue, fibrosis, fibrotic cells, tissue hyperplasia, tissue necrosis, granulation tissue, and mucoid impaction. The mucociliary clearance region can extend along a continuous mucociliary clearance path from a location immediately distal to the implant 6300 to a location immediately proximal to the implant 6300. In at least some cases, the mucociliary clearance region is maintained at an average width parallel to the longitudinal axis at least 10, 12, 14, 16 or more times greater than an average cross-sectional diameter of the wire perpendicular to the wire path of the implant 6300.

Part of maintaining airway patency can be reducing or eliminating excessive shifting of the implant 6300 during respiration. Relatedly, maintaining patency can include resisting elongation of the implant 6300 along the longitudinal axis during a full respiration cycle by the subject with a resisting force less than a force of friction between the implant 6300 and a wall of the bronchial tree at the treatment location. This feature alone or together with other features can reduce or prevent airway irritation and associated formation of granulation tissue and/or other response that may reduce airway patency during the maintenance period. In at least some cases, the implant maintains airway patency and/or other desirable therapeutic performance levels described herein during the maintenance period without the presence of a drug-eluting material between expandable structures of the implant and a wall of the bronchial tree at the treatment location.

### CONCLUSION

This disclosure is not intended to be exhaustive or to limit the present technology to the precise forms disclosed herein. Although specific embodiments are disclosed herein for illustrative purposes, various modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may be disclosed herein in the context of those embodiments, other embodiments may also exhibit such advantages, and not all embodiments need necessarily exhibit such advantages or other advantages disclosed herein to fall within the scope of the present technology. This disclosure and the associated technology can encompass other embodiments not expressly shown or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising,” “including,” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various structures. It should be understood that such terms do not denote absolute orientation. The word “diameter” as used herein does not require that the corresponding structure be circular. When used in the context of one or more structures arranged in a noncircular configuration, the word “diameter” means a maximum distance the structure or structures define in a given plane perpendicular to a longitudinal dimension. Similarly, the word “helix” as used herein does not require that the corresponding structure be a geometrically precise helix, but rather than the structure resembles a helix or that a person of ordinary skill in the art would otherwise recognize the structure to have helical characteristics. The word “wire” encompasses any suitable wire-like elongate structure, including structures made by shaping processes (e.g., drawing, casting, and extruding), additive processes (e.g., 3D printing), and subtractive processes (e.g., cutting from a workpiece). Furthermore, these structures can be of any suitable cross-sectional shape (not just round).

Reference herein to “one embodiment,” “an embodiment,” or similar phrases means that a particular feature, structure, operation, or characteristic described in connection with such phrases can be included in at least one embodiment of the present technology. Thus, such phrases as used herein are not necessarily all referring to the same embodiment. As used herein, the terms “generally,” “substantially,” “about,” and similar terms are terms of approximation and not terms of degree. These terms are intended to account for the inherent variations in measured or calculated values that would be recognized by those of ordinary skill in the art. The word “inventors” as used herein refers to at least one inventor. Unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Finally, it should be noted that various particular features, structures, operations, and characteristics of the embodiments described herein may be combined in any suitable manner in additional embodiments in accordance with the present technology.

I/We claim:

1. A delivery system for deploying an implant at a treatment location, wherein the implant comprises a tubular region with one or more interstitial regions, the delivery system comprising:
a flexible elongate member having an implant mounting surface, wherein the implant mounting surface comprises a conformable material configured to adapt to the one or more interstitial regions of the implant when the implant is radially collapsed on the implant mounting surface, thereby engaging the implant; and
a sheath at least partially covering the elongate member, wherein the sheath is movable relative to the elongate member to at least partially expose the implant mounting surface.

2. The delivery system of claim 1, wherein the conformable material extends fully circumferentially around a longitudinal axis of the elongate member.

3. The delivery system of claim 1 or 2, wherein the implant mounting surface is substantially smooth.

4. The delivery system of any one of claims 1-3, wherein the conformable material comprises a thermoplastic.

5. The delivery system of any one of claims 1-4, wherein the conformable material has a durometer between about 5A and about 75A.

6. The delivery system of any one of claims 1-5, wherein the implant mounting surface comprises a first segment comprising the conformable material and a second segment comprising the conformable material.

7. The delivery system of claim 6, wherein the first and second segments are axially spaced apart along a longitudinal axis of the elongate member.

8. The delivery system of claim 6 or 7, wherein the first and second segments are circumferentially spaced apart around a longitudinal axis of the elongate member.

9. The delivery system of any one of claims 1-8, further comprising a handle operably coupled to the sheath.

10. The delivery system of claim 9, wherein the handle comprises an actuator configured to move the sheath relative to the elongate member.

11. The delivery system of claim 9 or 10, wherein the handle comprises a lock configured to selectively fix an axial position of the sheath relative to the elongate member.

12. The delivery system of any one of claims 1-11, wherein the elongate member comprises an atraumatic tip.

13. The delivery system of any one of claims 1-12, wherein the sheath is a first sheath and the delivery system further comprises a second sheath at least partially covering the first sheath, wherein the second sheath is movable relative to the first sheath.

14. The delivery system of any one of claims 1-13, further comprising a second lock configured to couple the delivery system to a bronchoscope.

15. The delivery system of claim 14, wherein the second lock is configured to selectively limit an axial position of the elongate member relative to the bronchoscope.

16. A system comprising:
an implant comprising a tubular region with one or more interstitial regions; and
a delivery system comprising:
a flexible elongate member having an implant mounting surface, wherein the mounting surface comprises a conformable material; and
a sheath at least partially covering the elongate member, wherein the sheath is movable relative to the elongate member to at least partially expose the implant mounting surface,
wherein the implant is arranged on the mounting surface such that the conformable material adapts to the one or more interstitial regions, thereby engaging the implant.

17. The system of claim 16, wherein the implant is configured to transition from a radially compressed configuration to a radially expanded configuration.

18. The system of claim 17, wherein the implant is configured to transition from the radially compressed configuration to a radially expanded configuration without experiencing a change in implant length.

19. The system of claim 17 or 18, wherein the implant is configured to self-expand from the radially compressed configuration to the radially expanded configuration.

20. The system of any one of claims 16-19, wherein the tubular region comprises a wire extending along a continuous wire path turning about a longitudinal axis of the implant.

21. The system of any one of claims 16-20, wherein the one or more interstitial regions comprises an open helical region.

22. The system of any one of claims 16-21, wherein the conformable material extends fully circumferentially around a longitudinal axis of the elongate member.

23. The system of any one of claims 16-22, wherein the implant mounting surface is substantially smooth.

24. The system of any one of claims 16-23, wherein the conformable material comprises a thermoplastic.

25. The system of any one of claims 16-24, wherein the conformable material has a durometer between about 5A and about 75A.

26. The system of any one of claims 16-25, wherein the conformable material is configured to engage with at least one of a proximal portion and a distal portion of the implant.

27. The system of any one of claims 16-26, wherein the conformable material is configured to engage with an entire length of the implant.

28. The system of any one of claims 16-27, wherein the implant mounting surface is a first implant mounting surface, and the elongate member comprises a second implant mounting surface comprising a second conformable material.

29. The system of claim 28, wherein the first and second implant mounting surfaces are axially spaced apart along a longitudinal axis of the elongate member.

30. The system of any one of claims 16-29, wherein the delivery system further comprises a handle operably coupled to the sheath.

31. The system of claim 30, wherein the handle comprises an actuator configured to move the sheath relative to the elongate member.

32. The system of claim 30 or 31, wherein the handle comprises a lock configured to selectively fix an axial position of the sheath relative to the elongate member.

33. The system of any one of claims 16-32, wherein the elongate member comprises an atraumatic tip.

34. The system of any one of claims 16-33, wherein the sheath is a first sheath and the delivery system further comprises a second sheath at least partially covering the first sheath, wherein the first sheath is movable relative to the second sheath.

35. The system of any one of claims 16-34, wherein the delivery system comprises a second lock configured to couple the delivery system to a bronchoscope.

36. The system of claim 35, wherein the second lock is configured to selectively limit movement of the elongate member relative to the bronchoscope.

37. The system of any one of claims 16-36, further comprising a single-use bronchoscope.

38. A method for deploying an implant in a patient, wherein the implant comprises a tubular region with one or more interstitial regions and is configured to transition from a radially compressed configuration to a radially expanded configuration, the method comprising:
advancing, in the patient, a flexible elongate member having an implant mounting surface comprising a conformable material, wherein the implant is arranged on the mounting surface in the radially compressed configuration such that the conformable material adapts to the one or more interstitial regions, wherein the elongate member is at least partially covered by a sheath that is movable relative to the elongate member;
positioning a first end of the compressed implant at a first target location;
moving the sheath relative to the elongate member, thereby exposing the implant; and
allowing the implant to transition to the radially expanded configuration.

39. The method of claim 38, wherein advancing the elongate member in the patient comprises advancing the elongate member through a working channel of a bronchoscope.

40. The method of claim 39, further comprising selectively limiting movement of the elongate member relative to the bronchoscope while moving the sheath relative to the elongate member in the patient.

41. The method of claim 40, wherein selectively limiting movement of the sheath comprises limiting an axial position of the elongate member relative to the bronchoscope.

42. The method of any one of claims 39-41, further comprising allowing rotational movement of the sheath relative to the bronchoscope.

43. A delivery system for deploying an implant at a treatment location, the delivery system comprising:
a flexible elongate member having an implant mounting surface, wherein the implant mounting surface comprises an engagement means for securing the implant when the implant is radially collapsed on the implant mounting surface; and
a sheath at least partially covering the elongate member, wherein the sheath is movable relative to the elongate member to at least partially expose the implant mounting surface.

44. The delivery system of claim 43, wherein the engagement means comprises a conformable material configured to adapt to one or more interstitial regions of the implant when the implant is radially collapsed on the implant mounting surface, thereby engaging the implant.

45. The delivery system of claim 44, wherein the conformable material comprises a thermoplastic.

46. The delivery system of any one of claims 43-45, wherein the engagement means comprises a bioadhesive.

47. The delivery system of claim 46, wherein the bioadhesive comprises at least one of the group consisting of a synthetic polymer, a polysaccharide, cellulose, chitosan, and fibrin.

48. The delivery system of any one of claims 43-47, wherein the engagement means comprises a textured surface.

49. The delivery system of any one of claims 43-48, wherein the engagement means extends fully circumferentially around a longitudinal axis of the elongate member.

50. An implant configured to be deployed at a treatment location within a bronchial tree of a patient, the implant comprising:
a tubular structure comprising a wire extending along a continuous wire path, the tubular structure comprising:
a proximal end portion configured to be deployed at a first airway of the bronchial tree and exert a first chronic outward force; and
a distal end portion configured to be deployed at a second airway of the bronchial tree and exert a second chronic outward force, wherein a generation of the second airway is greater than a generation of the first airway,
wherein the second chronic outward force is greater than the first chronic outward force.

51. The implant of claim 50, wherein the tubular structure comprises an intermediate portion between the proximal end portion and the distal end portion, wherein the tubular structure is configured to exert a variable chronic outward force along its length.

52. The implant of claim 50 or 51, wherein the variable chronic outward force ranges from the first chronic outward force to the second chronic outward force.

53. The implant of any one of claims 50-52, wherein the second chronic outward force is between about two and about four times greater than the first chronic outward force.

54. The implant of any one of claims 50-53, wherein the second chronic outward force is about three times greater than the first chronic outward force.

55. The implant of any one of claims 50-54, wherein the second chronic outward force is about 15N, and the first chronic outward force is about 5N.

56. The implant of any one of claims 50-55, wherein the tubular structure is configured to transition from a radially compressed configuration to a radially expanded configuration.

57. The implant of any one of claims 50-56, wherein the second chronic outward force is sufficient to cause dilation of the second airway to at least 2 times the native diameter of the second airway.

58. A system for selecting a length of an endobronchial implant to be placed in a patient, the system comprising:
a flexible elongate member configured to be inserted through a lumen of a device,
wherein the elongate member comprises a plurality of markers arranged along a longitudinal axis of the elongate member, wherein at least a portion of the plurality of markers are evenly distributed along the longitudinal axis, or at least a portion of the plurality of markers correspond to predetermined available lengths of the endobronchial implant, or both.

59. The system of claim 58, wherein the plurality of markers are arranged on a distal portion of the elongate member.

60. The system of claim 58, wherein the plurality of markers are arranged on a proximal portion of the elongate member.

61. The system of any one of claims 58-60, wherein at least a portion of the plurality of markers comprise a radiopaque material.

62. The system of any one of claims 58-61, wherein the elongate member is a guidewire.

63. The system of any one of claims 58-61, wherein the elongate member is a probe.

64. The system of any one of claims 58-61, wherein the elongate member is a sheath defining a lumen.

65. The system of claim 64, wherein the elongate member is a first elongate member and the sheath is a first sheath, wherein the first sheath is coupleable to an implant delivery system comprising:
a second flexible elongate member having an implant mounting surface; and
a second sheath at least partially covering the second elongate member, wherein the second sheath is movable relative to the second elongate member to at least partially expose the implant mounting surface.

66. The system of claim 65, wherein the first sheath is removably coupleable to a handle of the implant delivery system.

67. The system of any one of claims 58-F9, wherein the elongate member comprises a proximity sensor.

68. A method for selecting a length of an endobronchial implant to be placed in a patient, the method comprising:
navigating a device toward a target airway in the patient;
advancing an elongate member through a lumen of the device such that a distal end of the elongate member is distally beyond a distal end of the device;
measuring a length of the target airway using a plurality of markers on the elongate member; and
selecting a length of an endobronchial implant based on the measured length of the target airway.

69. The method of claim 68, wherein the device is a bronchoscope or robotic catheter.

70. The method of claim 69, wherein the device is a single-use bronchoscope.

71. The method of any one of claims 68-70, wherein advancing the elongate member comprises advancing the elongate member until the distal end of the elongate member is located at a desired location of a distal end of the endobronchial implant.

72. The method of claim 71, wherein the elongate member is a guidewire.

73. The method of any one of claims 68-70, wherein advancing the elongate member comprises advancing the elongate member until the distal end of the elongate member is adjacent to pleura.

74. The method of claim 73, further comprising proximally retracting the elongate member until the distal end of the elongate member is located at a desired location of a distal end of the endobronchial implant.

75. The method of any one of claims 68-74, wherein the plurality of markers are arranged on a distal portion of the elongate member.

76. The method of any one of claims 68-75, wherein the plurality of markers are arranged on a proximal portion of the elongate member.

77. The method of any one of claims 68-76, wherein at least a portion of the plurality of markers are evenly distributed along a longitudinal axis of the elongate member.

78. The method of any one of claims 68-77, wherein at least a portion of the plurality of markers correspond to predetermined available lengths of the endobronchial implant.

79. The method of any one of claims 68-78, wherein at least a portion of the plurality of markers comprise a radiopaque material.

80. The method of any one of claims 68-79, further comprising measuring a distance between the distal end of the elongate member and pleura.

81. The method of claim 80, wherein the distance between the distal end of the elongate member and pleura is measured with a sensor.

82. A delivery system for deploying an implant at a treatment location, comprising:
a flexible elongate member having an implant mounting surface configured to receive the implant;
a sheath at least partially covering the elongate member, wherein the sheath is movable relative to the elongate member to at least partially expose the implant mounting surface; and
a sensor configured to detect proximity of a distal end of the delivery system to a tissue wall.

83. The delivery system of claim 82, wherein the sensor is arranged on a distal end of the elongate member.

84. The delivery system of claim 82, wherein the sensor is arranged on a distal end of the sheath.

85. The delivery system of any one of claims 82-84, wherein the sensor comprises an ultrasonic sensor.

86. The delivery system of any one of claims 82-85, wherein the sensor comprises an infrared sensor.

87. A method for deploying an implant at a treatment location, the method comprising:
advancing, in an airway of the patient, a delivery system comprising a flexible elongate member having an implant mounted thereon, a sheath that is movable relative to the elongate member, and a proximity sensor arranged on a distal end of the delivery system;
detecting a position of the distal end of the delivery system relative to a pleura of the patient using the proximity sensor;
positioning the implant at a desired target location without contacting the pleura with the delivery system; and
moving the sheath relative to the elongate member to expose the implant at the desired target location.

88. The method of claim 87, wherein advancing the delivery system comprises advancing the delivery system through a bronchoscope or robotic catheter.

89. The method of claim 88, wherein the bronchoscope is a single-use bronchoscope.

90. The method of any one of claims 87-89, wherein moving the sheath to expose the implant allows the implant to transition from a radially compressed configuration to a radially expanded configuration.

91. The method of any one of claims 87-90, wherein moving the sheath comprises proximally retracting the sheath.

92. A method for deploying an implant at a treatment location, the method comprising:
advancing, in an airway of the patient, a delivery system comprising a flexible elongate member having an implant mounted thereon and a sheath that is movable relative to the elongate member;
advancing the delivery system until a distal end of the delivery system touches a pleura of the patient;
retracting the delivery system until the implant is at a desired target location; and
moving the she relative to the elongate member to expose the implant at the desired target location.

93. The method of claim 92, wherein advancing the delivery system comprises advancing the delivery system through a bronchoscope or robotic catheter.

94. The method of claim 93, wherein the bronchoscope is a single-use bronchoscope.

95. The method of any one of claims 92-94, wherein moving the sheath to expose the implant allows the implant to transition from a radially compressed configuration to a radially expanded configuration.

96. The method of any one of claims 92-95, wherein moving the sheath comprises proximally retracting the sheath.
